(19) United States
(12) Patent Application Publication
(10) Pub. No.: US 2024/0188427 A1
(43) Pub. Date: Jun. 6, 2024

(54) ORGANIC COMPOUND, OPTO-ELECTRONIC DEVICE INCLUDING THE SAME, ELECTRONIC APPARATUS INCLUDING THE OPTO-ELECTRONIC DEVICE, AND ELECTRONIC DEVICE INCLUDING THE ELECTRONIC APPARATUS

(71) Applicant: Samsung Display Co., LTD., Yongin-si (KR)

(72) Inventors: Seran Kim, Yongin-si (KR); Hoilim Kim, Yongin-si (KR); Dongsun Yoo, Yongin-si (KR); Junyoung Lee, Yongin-si (KR)

(21) Appl. No.: 18/502,508

(22) Filed: Nov. 6, 2023

(30) Foreign Application Priority Data
Nov. 8, 2022 (KR) 10-2022-0148133

(51) Int. Cl. H10K 85/60; G02F 1/1335

(52) U.S. Cl. CPC H10K 85/655; G02F 1/133514; H10K 85/653; H10K 85/654; H10K 85/6572; H10K 85/6574; H10K 85/6576

(57) ABSTRACT

Provided is an opto-electronic device, an organic compound, an electronic apparatus and an electronic device, and the opto-electronic device includes a first electrode, a second electrode facing the first electrode, a photoactive layer arranged between the first electrode and the second electrode, and an organic compound represented by Formula 1, wherein, in Formula 1, CY1 is a group represented by Formula 2, and CY2 is a group represented by Formula 3, wherein details of Formulae 1 to 3 are as described herein.

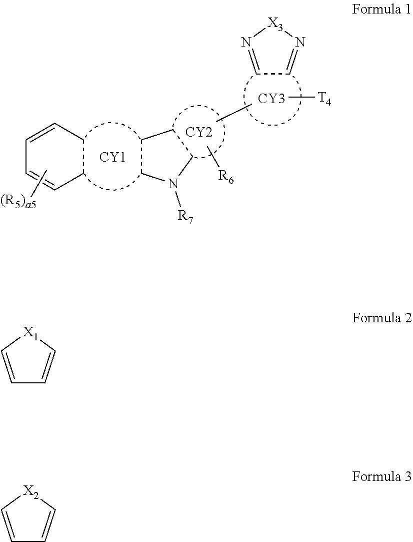

### CROSS-REFERENCE TO RELATED APPLICATION

This application is claims priority to and the benefit of Korean Patent Application No. 10-2022-0148133, filed on Nov. 8, 2022, in the Korean Intellectual Property Office, the entire content of which is incorporated by reference herein in its entirety.

### BACKGROUND

### 1. Field

One or more embodiments of the present disclosure relate to an organic compound, an opto-electronic device including the same, an electronic apparatus including the opto-electronic device, and an electronic device including the electronic apparatus.

### 2. Description of the Related Art

Opto-electronic devices are devices that convert light energy or a light signal into electrical energy or an electrical signal. Examples of opto-electronic devices include photovoltaic cells or solar cells that convert light energy into electrical energy, photodetectors or light sensors that detect light energy and convert the detected light energy into an electrical signal, and the like.

Electronic apparatuses including opto-electronic devices and organic light-emitting devices have been developed. In an example, light emitted from an organic light-emitting device may be reflected by an object (e.g., a finger of a user) in contact with an electronic apparatus, and then incident on an opto-electronic device. As the opto-electronic device detects incident light energy and converts the detected incident light energy into an electrical signal, it may be recognized that the object is in contact with the electronic apparatus. The opto-electronic device may be used as a fingerprint recognition sensor or the like.

### SUMMARY

One or more embodiments of the present disclosure include an organic compound having improved light absorption efficiency for a set or specific wavelength, and an opto-electronic device having high light absorption efficiency by including the organic compound. In addition, one or more embodiments include a high-quality electronic apparatus and a high-quality electronic device that include the opto-electronic device.

Additional aspects of embodiments will be set forth in part in the description which follows and, in part, will be apparent from the description, or may be learned by practice of the presented embodiments of the disclosure.

According to one or more embodiments, an opto-electronic device includes a first electrode, a second electrode facing the first electrode, a photoactive layer arranged between the first electrode and the second electrode, and an organic compound represented by Formula 1:

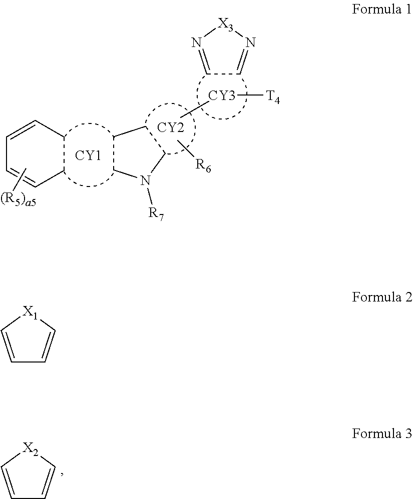

wherein, in Formulae 1 to 3,

CY1 may be a group represented by Formula 2,

CY2 may be a group represented by Formula 3,

CY3 may be a C₃-C₆₀ carbocyclic group or a C₁-C₆₀ heterocyclic group,

X₁ may be O, S, Se, N(R₁), or P(═O)R₁,

X₂ may be O, S, Se, N(R₂), or P(═O)R₂,

X₃ may be O, S, Se, N(R₃), or P(═O)R₃,

T₄ may be *-(L₄)_{b4}-(R₄)ₐ₄,

L₄ may be a single bond, a C₁-C₆₀ alkylene group unsubstituted or substituted with at least one R₁₀ₐ, a C₂-C₆₀ alkenylene group unsubstituted or substituted with at least one R₁₀ₐ, or a C₂-C₆₀ alkynylene group unsubstituted or substituted with at least one R₁₀ₐ,

b4 may be an integer from 0 to 3,

R₁ to R₇ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),

a4 and a5 may each independently be an integer from 0 to 4,

R₁₀ₐ may be:

deuterium, —F, —Cl, —Br, —I, a hydroxyl group, a cyano group, or a nitro group,

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof,

a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof, or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),

Q₁ to Q₃, Q₁₁ to Q₁₃, Q₂₁ to Q₂₃, and Q₃₁ to Q₃₃ may each independently be hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, a hydroxyl group, a cyano group, a nitro group, or any combination thereof, a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a C₃-C₆₀ carbocyclic group, a C₁-C₆₀ heterocyclic group, or any combination thereof, a C₇-C₆₀ arylalkyl group, or a C₂-C₆₀ heteroarylalkyl group, and

* indicates a binding site to CY3.

According to one or more embodiments, an electronic apparatus includes the opto-electronic device.

According to one or more embodiments, an electronic device includes the electronic apparatus.

According to one or more embodiments, provided is the organic compound represented by Formula 1.

### BRIEF DESCRIPTION OF THE DRAWINGS

The above and other aspects and features of certain embodiments of the disclosure will be more apparent from the following description taken in conjunction with the accompanying drawings, in which:

FIG. 1 is a schematic plan view of an opto-electronic device according to one or more embodiments;

FIG. 2 is a schematic cross-sectional view of a light-emitting device included in an electronic apparatus according to one or more embodiments;

FIG. 3 is a schematic cross-sectional view of an electronic apparatus according to one or more embodiments;

FIG. 4 is a schematic cross-sectional view of an electronic apparatus according to one or more embodiments;

FIG. 5 is a schematic perspective view of an electronic device including a light-emitting device according to one or more embodiments;

FIG. 6 is a schematic view of the exterior of a vehicle as an electronic device including a light-emitting device according to one or more embodiments; and

FIGS. 7A to 7C are schematic views of the interior of a vehicle according to various embodiments.

### DETAILED DESCRIPTION

Reference will now be made in more detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of embodiments of the present description. As used herein, the term “and/or” includes any and all combinations of one or more of the associated listed items. Throughout the disclosure, the expression “at least one of a, b and c” indicates only a, only b, only c, both a and b, both a and c, both b and c, all of a, b, and c, or variations thereof.

An aspect of embodiments of the disclosure provides an opto-electronic device including a first electrode, a second electrode facing the first electrode, a photoactive layer arranged between the first electrode and the second electrode, and an organic compound represented by Formula 1.

In one or more embodiments, the first electrode may be an anode. The second electrode may be a cathode. The emission layer may include a dopant and a host, and may emit light. The dopant and the host may each be as described herein.

In one or more embodiments, the opto-electronic device may further include a hole transport region arranged between the first electrode and the photoactive layer and an electron transport region arranged between the photoactive layer and the second electrode. The photoactive layer may include the organic compound.

In one or more embodiments, the photoactive layer may include a first layer adjacent to the hole transport region and a second layer adjacent to the electron transport region. The first layer may include the organic compound.

For example, the second layer may include fullerene.

For example, the first layer may be referred to as a P-type layer or an electron donor layer. The second layer may be referred to as an N-type layer or an electron acceptor layer.

In one or more embodiments, the first layer may be in direct contact with the hole transport region. The second layer may be in direct contact with the electron transport region.

Another aspect of embodiments of the disclosure provides an electronic apparatus including the opto-electronic device.

In one or more embodiments, the electronic apparatus may further include a thin-film transistor electrically connected to the first electrode, an emission layer arranged between the first electrode and the second electrode and not overlapping the photoactive layer, and a color filter, a color conversion layer, a touch screen layer, a polarizing layer, or any combination thereof.

For example, a light-emitting device may include the first electrode, the emission layer, and the second electrode. The light-emitting device may further include a hole transport region arranged between the first electrode and the emission layer and overlapping the emission layer, and an electron transport region arranged between the second electrode and the emission layer and overlapping the emission layer.

In more detail, each of the hole transport region and the electron transport region may be a common layer, and may overlap both the photoactive layer and the emission layer.

In one or more embodiments, the hole transport region may include a hole injection layer, a hole transport layer, an emission auxiliary layer, an electron blocking layer, or any combination thereof. The electron transport region may include a buffer layer, a hole blocking layer, an electron control layer, an electron transport layer, an electron injection layer, or any combination thereof.

Another aspect of embodiments of the disclosure provides an electronic device including the electronic apparatus. The electronic device may be one selected from a flat panel display, a curved display, a computer monitor, a medical monitor, a television, a billboard, an indoor or outdoor lighting and/or signal light, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a microdisplay, a three-dimensional (3D) display, a virtual or augmented-reality display, a vehicle, a video wall including multiple displays tiled together, a theater or stadium screen, a phototherapy device, and a signboard.

Another aspect of embodiments of the disclosure provides an organic compound represented by Formula 1:

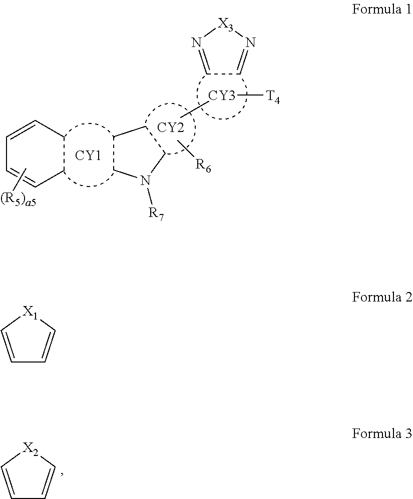

wherein, in Formulae 1 to 3,

CY1 may be a group represented by Formula 2,

CY2 may be a group represented by Formula 3,

CY3 may be a C₃-C₆₀ carbocyclic group or a C₁-C₆₀ heterocyclic group,

X₁ may be O, S, Se, N(R₁), or P(═O)R₁,

X₂ may be O, S, Se, N(R₂), or P(═O)R₂,

X₃ may be O, S, Se, N(R₃), or P(═O)R₃,

T₄ may be *-(L₄)_{b4}-(R₄)ₐ₄,

L₄ may be a single bond, a C₁-C₆₀ alkylene group unsubstituted or substituted with at least one R₁₀ₐ, a C₂-C₆₀ alkenylene group unsubstituted or substituted with at least one R₁₀ₐ, or a C₂-C₆₀ alkynylene group unsubstituted or substituted with at least one R₁₀ₐ,

b4 may be an integer from 0 to 3,

R₁ to R₇ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),

a4 and a5 may each independently be an integer from 0 to 4,

R₁₀ₐ may be:

deuterium, —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;

a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),

Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group, a C₂-C₆₀ alkenyl group, a C₂-C₆₀ alkynyl group, a C₁-C₆₀ alkoxy group, each unsubstituted or substituted with deuterium, —F, a hydroxyl group, a cyano group, a nitro group, or any combination thereof; a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a C₃-C₆₀ carbocyclic group, a C₁-C₆₀ heterocyclic group, or any combination thereof; a C₇-C₆₀ arylalkyl group; or a C₂-C₆₀ heteroarylalkyl group, and

* may indicate a binding site to CY3.

In one or more embodiments, the organic compound may be represented by one selected from Formulae 2-1 and 2-2:

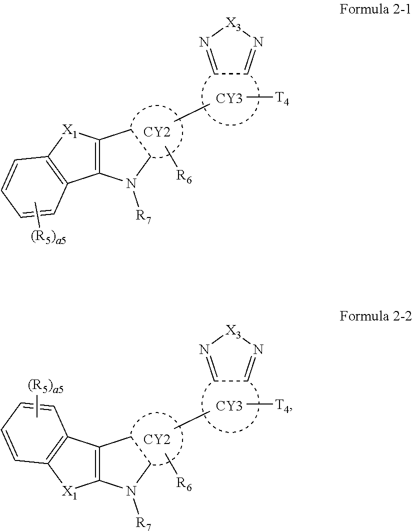

wherein, in Formulae 2-1 and 2-2,

CY2, CY3, X₁ to X₃, T₄, L₄, b4, R₁ to R₇, a4, and a5 may each be as defined herein.

For example, the organic compound may be represented by Formula 2-1.

In one or more embodiments, the organic compound may be represented by one selected from Formulae 3-1 to 3-4:

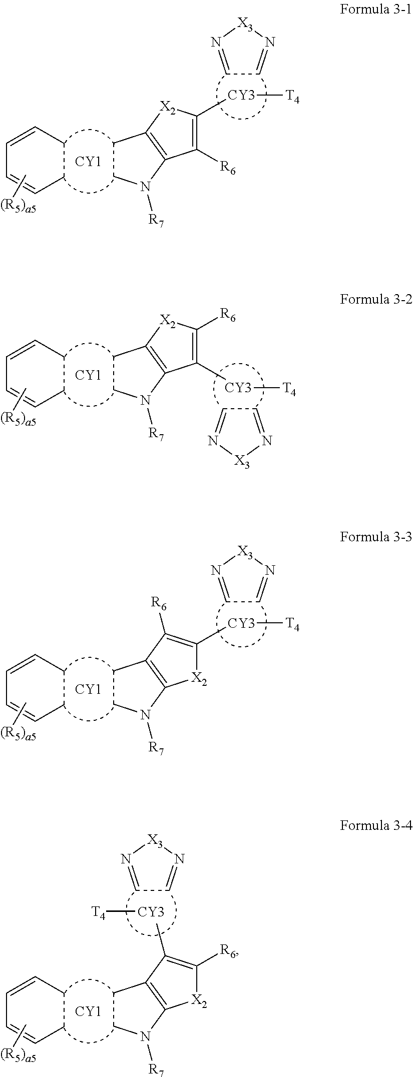

wherein, in Formulae 3-1 to 3-4,

CY1, CY3, X₁ to X₃, T₄, L₄, b4, R₁ to R₇, a4, and a5 may each be as defined herein.

For example, the organic compound may be represented by Formula 3-1.

In one or more embodiments, the organic compound may be represented by one selected from Formulae 1-1 to 1-8:

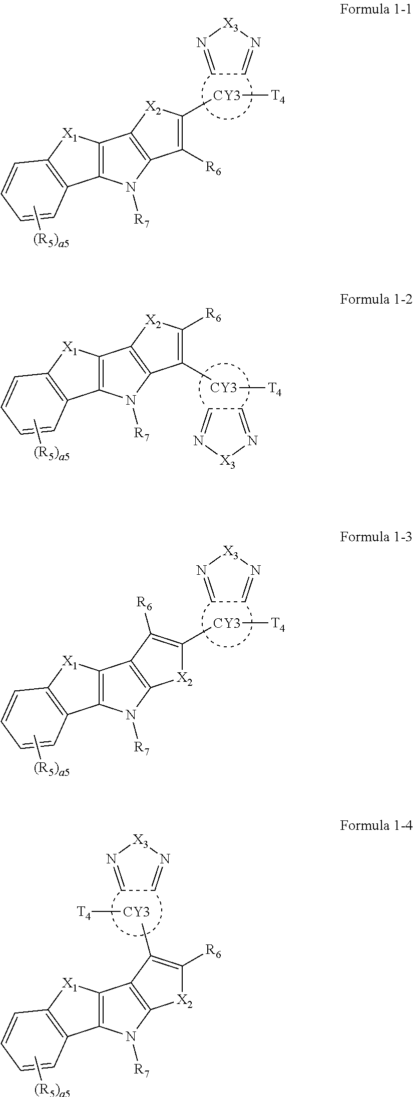

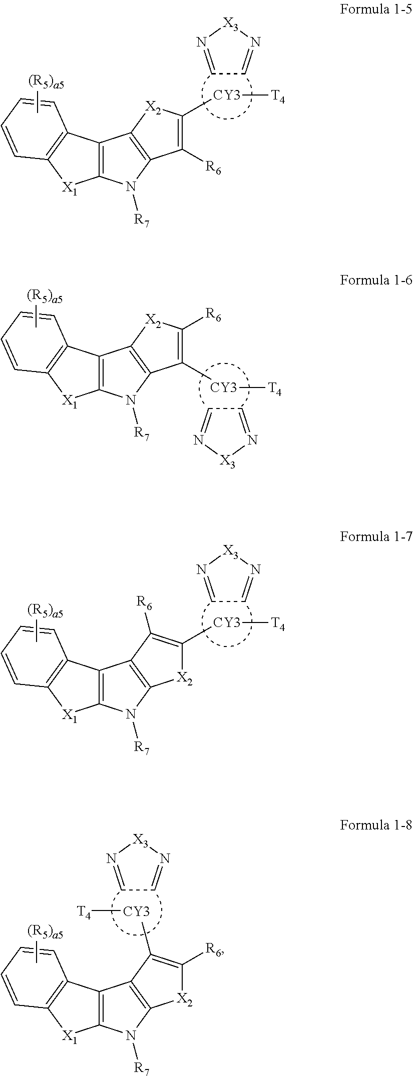

wherein, in Formulae 1-1 to 1-8,

CY3, X₁ to X₃, T₄, L₄, b4, R₁ to R₇, a4, and a5 may each be as defined herein.

For example, the organic compound may be represented by Formula 1-1.

In one or more embodiments, CY3 in Formula 1 may be a benzene group, a naphthalene group, a pyridine group, a pyrazine group, a pyrimidine group, a pyridazine group, a quinoline group, or an isoquinoline group.

For example, CY3 in Formula 1 may be a benzene group or a naphthalene group. For example, CY3 in Formula 1 may be a benzene group.

In one or more embodiments, Formula 1 may be represented by one selected from Formulae 4-1 to 4-6:

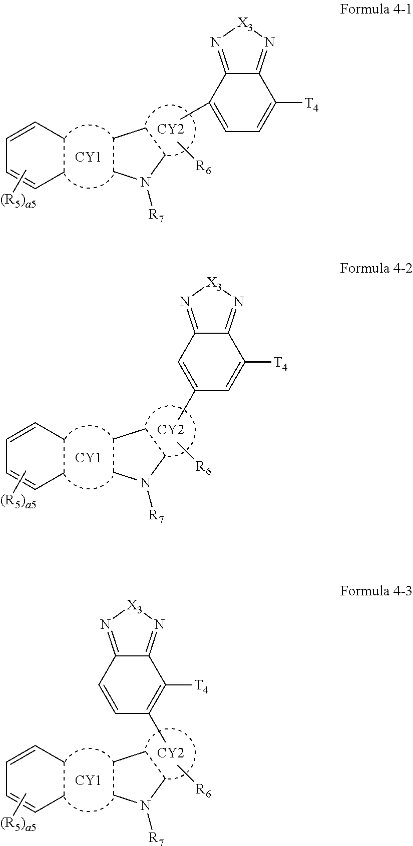

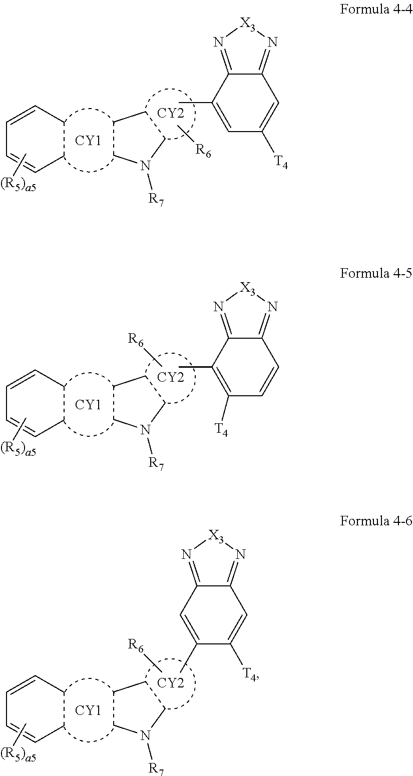

wherein, in Formulae 4-1 to 4-6,

CY1, CY2, X₁ to X₃, T₄, L₄, b4, R₁ to R₇, a4, and a5 may each be as defined herein.

In one or more embodiments, X₁ to X₃ in Formulae 1 to 3 may each independently be O, S, or Se.

In one or more embodiments, X₁ and X₂ in Formulae 1 to 3 may be identical to each other.

For example, X₁ to X₃ in Formulae 1 to 3 may be identical to each other.

For example, X₁ to X₃ in Formulae 1 to 3 may each be O, S, or Se.

For example, in Formulae 1 to 3, X₁ and X₂ may be identical to each other, and X₃ may be different from X₁ and X₂.

For example, in Formulae 1 to 3, X₁ and X₂ may be identical to each other, X₃ may be different from X₁ and X₂, and X₃ may be an element of a higher period in the Periodic Table of Elements than X₁ and X₂.

For example, in Formulae 1 to 3, when X₁ and X₂ are each O, X₃ may be S or Se. For example, in Formulae 1 to 3, when X₁ and X₂ are each S, X₃ may be Se.

For example, in Formulae 1 to 3, X₁ and X₂ may be identical to each other, X₃ may be different from X₁ and X₂, and X₃ may be an element of a lower period in the Periodic Table of Elements than X₁ and X₂.

For example, in Formulae 1 to 3, when X₁ and X₂ are each Se, X₃ may be O or S. For example, in Formulae 1 to 3, when X₁ and X₂ are each S, X₃ may be O.

In one or more embodiments, L₄ in Formula 1 may be a single bond or a C₂-C₆₀ alkenylene group unsubstituted or substituted with at least one R₁₀ₐ.

For example, L₄ may be a single bond or a C₂-C₁₀ alkenylene group unsubstituted or substituted with at least one R₁₀ₐ. For example, L₄ may be a single bond or a vinylidene group unsubstituted or substituted with at least one R₁₀ₐ.

In one or more embodiments, b4 in Formula 1 may be 0 or 1. For example, when b4 is 0, (L₄)_{b4} may be a single bond.

In one or more embodiments, R₁ to R₇ in Formulae 1 to 3 may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, or a C₂-C₆₀ alkynyl group unsubstituted or substituted with at least one R₁₀ₐ.

In one or more embodiments, R₄ to R₇ in Formulae 1 to 3 may each independently be hydrogen, deuterium, —F, —Cl, —Br, —I, a hydroxyl group, a cyano group, a C₁-C₆₀ alkyl group unsubstituted or substituted with at least one R₁₀ₐ, or a C₂-C₆₀ alkenyl group unsubstituted or substituted with at least one R₁₀ₐ.

For example, R₄ in Formula 1 may be a cyano group or a C₂-C₆₀ alkenyl group unsubstituted or substituted with at least one R₁₀ₐ. For example, R₄ in Formula 1 may be a cyano group or a C₂-C₆₀ alkenyl group substituted with at least one cyano group. For example, R₄ in Formula 1 may be a cyano group or a C₂-C₆₀ alkenyl group substituted with two or more cyano groups.

For example, R₇ in Formula 1 may be hydrogen, deuterium, or a C₁-C₆₀ alkyl group unsubstituted or substituted with at least one R₁₀ₐ.

In one or more embodiments, a4 in Formula 1 may be an integer from 0 to 2.

In one or more embodiments, T₄ may include two or more cyano groups.

In one or more embodiments, T₄ in Formula 1 may be represented by Formula 5:

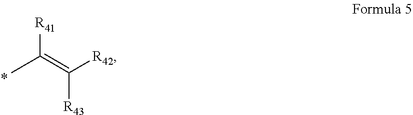

wherein, in Formula 5,

R₄₁ to R₄₃ may each independently be as defined herein in connection with R₄, and * may indicate a binding site to CY3.

For example, R₄₂ and R₄₃ in Formula 5 may be identical to each other. For example, R₄₂ and R₄₃ in Formula 5 may each be a cyano group.

In one or more embodiments, a maximum absorption wavelength of the organic compound may be in a range of about 600 nm to about 660 nm. In more detail, the maximum absorption wavelength of the organic compound may be in a range of about 615 nm to about 645 nm.

In one or more embodiments, a molecular weight of the organic compound may be 700 g/mol or less. For example, the organic compound may be a monomer.

In one or more embodiments, the organic compound may be one selected from Compounds 1 to 32:

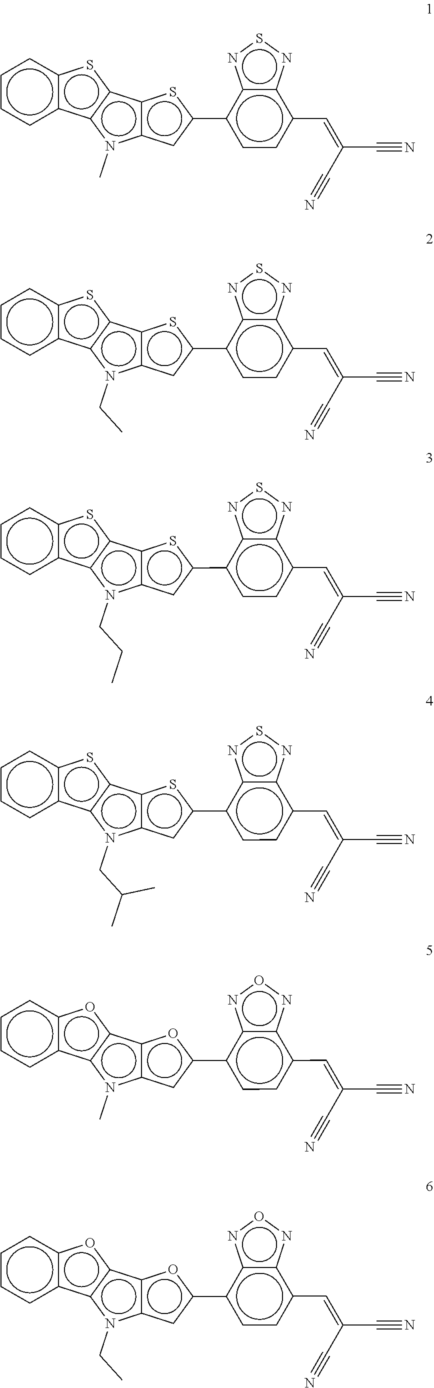

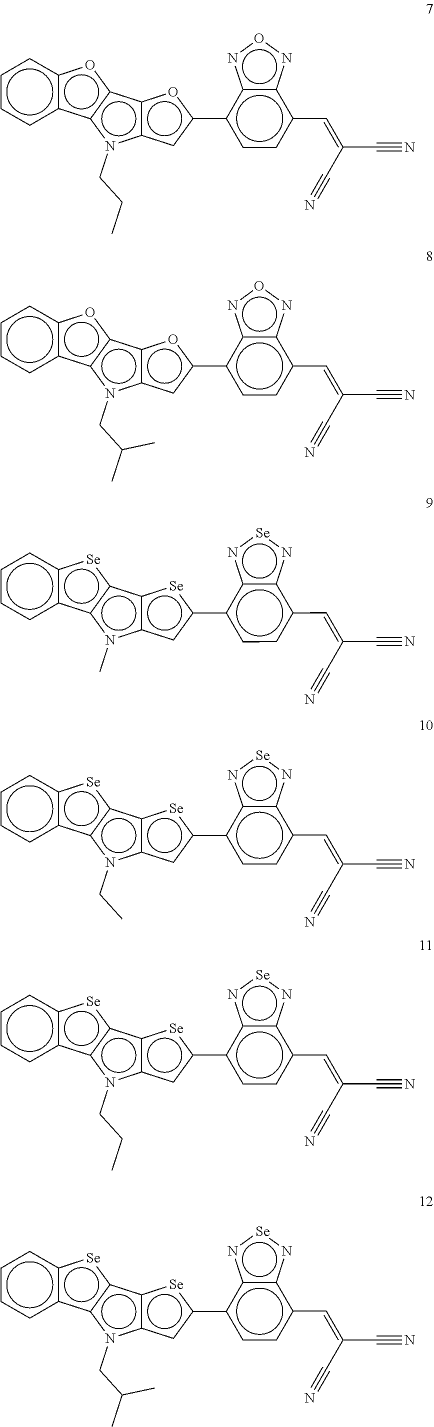

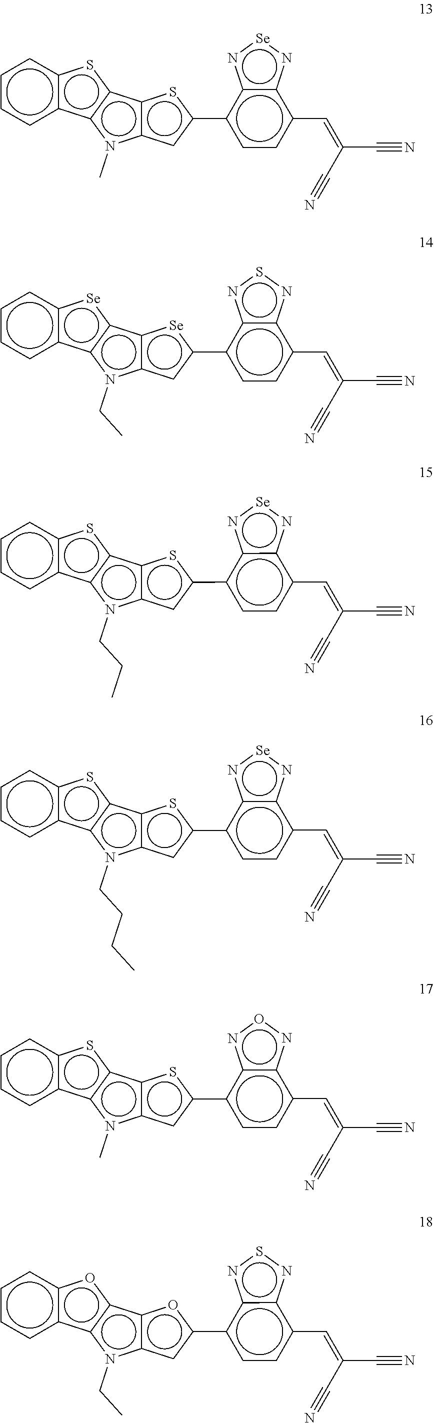

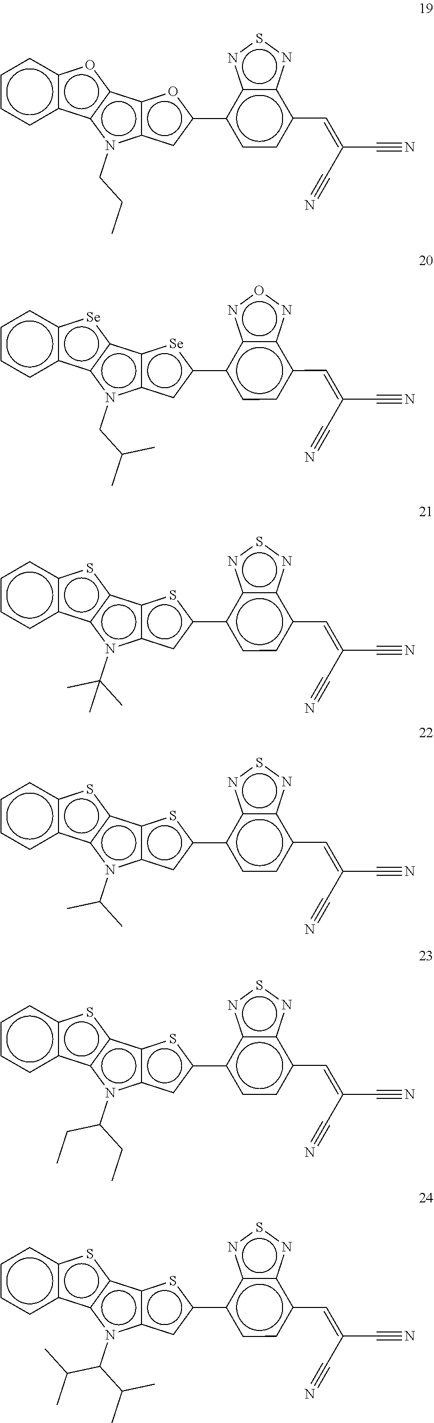

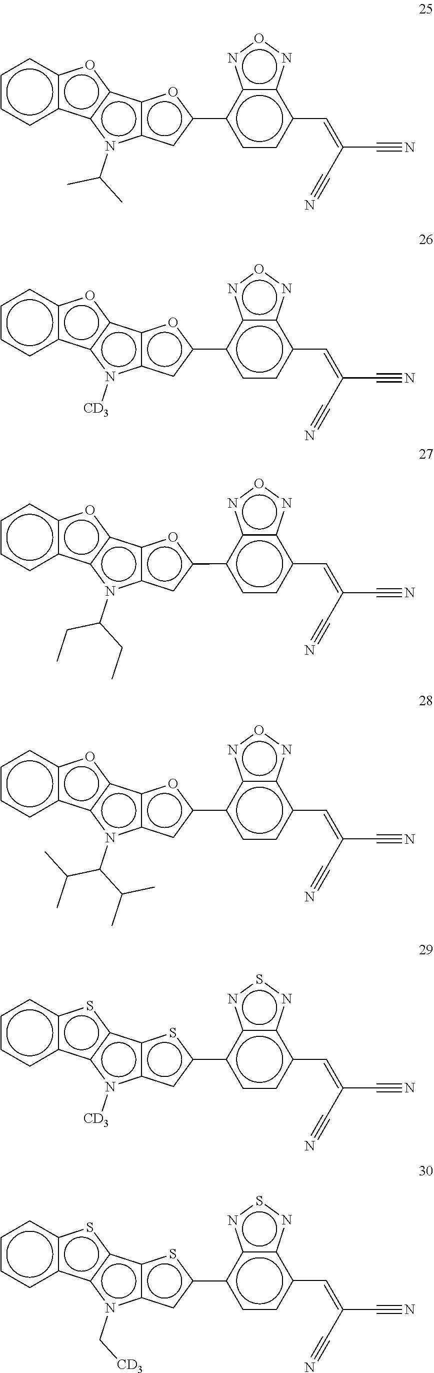

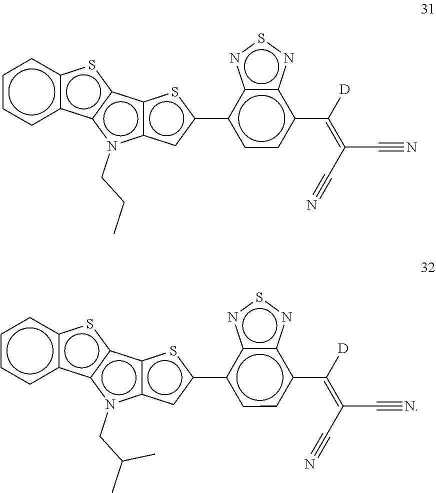

The organic compound represented by Formula 1 may have excellent light absorption efficiency at a set or specific wavelength (e.g., a central region of a red wavelength). For example, the organic compound may have a maximum absorption coefficient in a wavelength range of about 600 nm to about 660 nm. Accordingly, the organic compound represented by Formula 1 may have excellent light absorption efficiency for a red wavelength, and an opto-electronic device including the organic compound may have high light absorption efficiency for red wavelength.

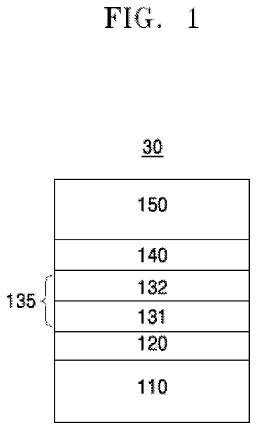

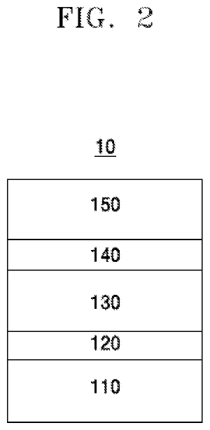

### Description of FIGS. 1 and 2

FIG. 1 is a schematic view of an opto-electronic device 30 according to one or more embodiments. The opto-electronic device 30 may include a first electrode 110, a hole transport region 120, a photoactive layer 135, an electron transport region 140, and a second electrode 150.

FIG. 2 is a schematic view of a light-emitting device 10. The light-emitting device 10 may include a first electrode 110, a hole transport region 120, an emission layer 130, an electron transport region 140, and a second electrode 150.

Hereinafter, the structures of the opto-electronic device 30 and the light-emitting device 10 according to embodiments and methods of manufacturing the opto-electronic device 30 and the light-emitting device 10 will be described with reference to FIGS. 1 and 2.

### First Electrode 110

In FIG. 1, a substrate may be additionally arranged under the first electrode 110 or on the second electrode 150. As the substrate, a glass substrate and/or a plastic substrate may be used. In one or more embodiments, the substrate may be a flexible substrate, and may include plastics having excellent heat resistance and durability, such as polyimide, polyethylene terephthalate (PET), polycarbonate, polyethylene naphthalate, polyarylate (PAR), polyetherimide, or any combination thereof.

The first electrode 110 may be formed by, for example, depositing and/or sputtering a material for forming the first electrode 110 on the substrate. When the first electrode 110 is an anode, a material for forming the first electrode 110 may be a high-work function material that facilitates injection of holes.

The first electrode 110 may be a reflective electrode, a semi-transmissive electrode, or a transmissive electrode. When the first electrode 110 is a transmissive electrode, a material for forming the first electrode 110 may include indium tin oxide (ITO), indium zinc oxide (IZO), tin oxide (SnO₂), zinc oxide (ZnO), or any combination thereof. In one or more embodiments, when the first electrode 110 is a semi-transmissive electrode or a reflective electrode, a material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combination thereof.

The first electrode 110 may have a single-layered structure consisting of a single layer or a multi-layered structure including a plurality of layers. For example, the first electrode 110 may have a three-layered structure of ITO/Ag/ITO.

### Hole Transport Region 120

The hole transport region 120 may have i) a single-layered structure consisting of a single layer consisting of a single material, ii) a single-layered structure consisting of a single layer consisting of a plurality of different materials, or iii) a multi-layered structure including a plurality of layers including different materials.

The hole transport region 120 may include a hole injection layer, a hole transport layer, an emission auxiliary layer, an electron blocking layer, or any combination thereof.

For example, the hole transport region 120 may have a multi-layered structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron blocking layer structure, the layers of each structure being sequentially stacked from the first electrode 110.

The hole transport region 120 may include a compound represented by Formula 201, a compound represented by Formula 202, or any combination thereof:

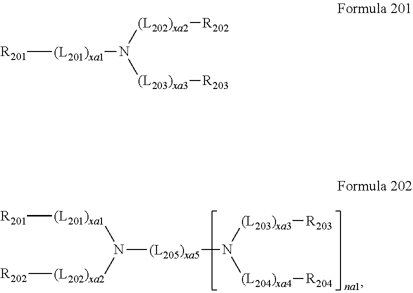

wherein, in Formulae 201 and 202,

L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

L₂₀₅ may be *—O—*′, *—S—*′, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,

xa1 to xa4 may each independently be an integer from 0 to 5,

xa5 may be an integer from 1 to 10,

R₂₀₁ to R₂₀₄ and Q₂₀₁ may each independently be a C₃-C₆₀ carbocyclic group unsubstituted or substituted with at least one R₁₀ₐ, or a C₁-C₆₀ heterocyclic group unsubstituted or substituted with at least one R₁₀ₐ,

R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group (e.g., a carbazole group, etc.) unsubstituted or substituted with at least one R₁₀ₐ (e.g., Compound HT16),

R₂₀₃ and R₂₀₄ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group unsubstituted or substituted with at least one R₁₀ₐ, and

na1 may be an integer from 1 to 4.

For example, each of Formulae 201 and 202 may include at least one selected from groups represented by Formulae CY201 to CY217.

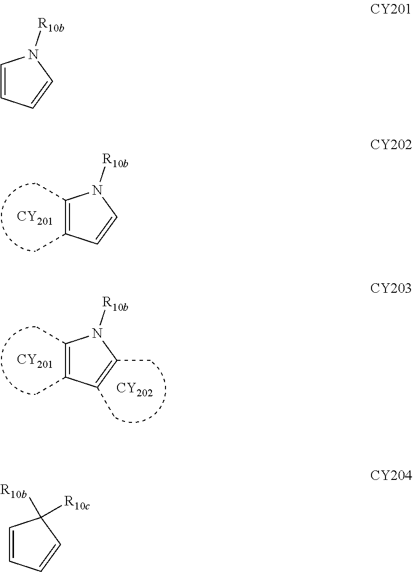

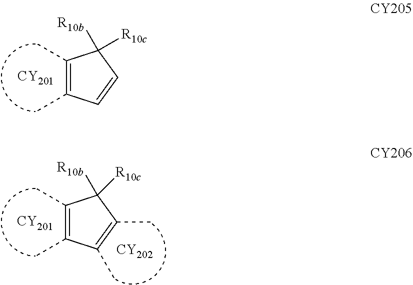

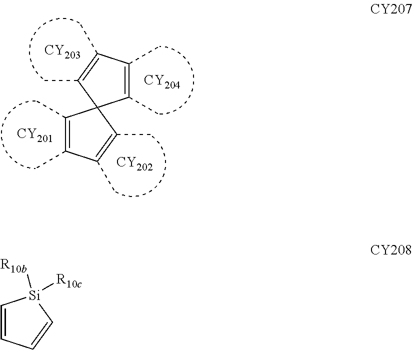

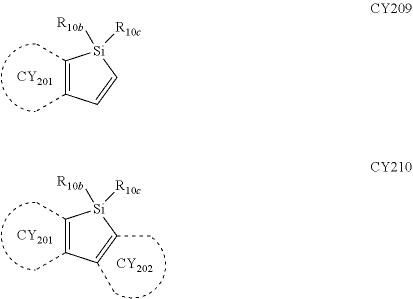

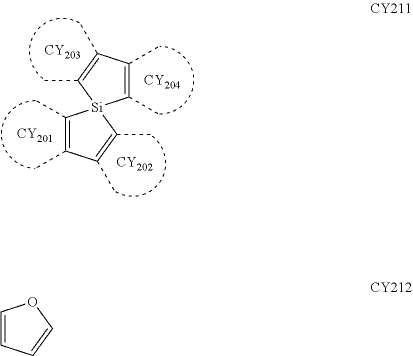

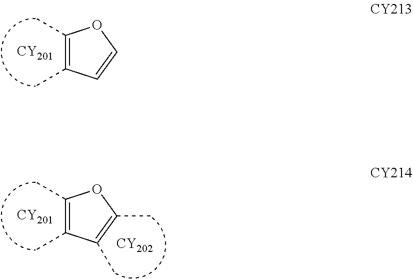

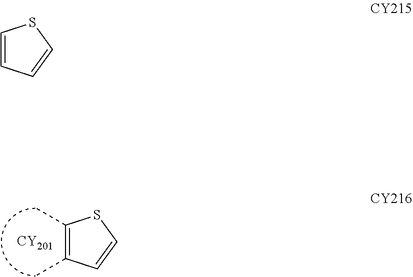

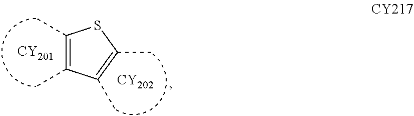

wherein, in Formulae CY201 to CY217, R_{10b} and R_{10c} may each be as defined herein in connection with R₁₀ₐ, ring CY201 to ring CY204 may each independently be a C₃-C₂₀ carbocyclic group or a C₁-C₂₀ heterocyclic group, and at least one hydrogen in Formulae CY201 to CY217 may be unsubstituted or substituted with R₁₀ₐ.

In one or more embodiments, ring CY201 to ring CY204 in Formulae CY201 to CY217 may each independently be a benzene group, a naphthalene group, a phenanthrene group, or an anthracene group.

In one or more embodiments, each of Formulae 201 and 202 may include at least one selected from groups represented by Formulae CY201 to CY203.

In one or more embodiments, Formula 201 may include at least one selected from groups represented by Formulae CY201 to CY203 and at least one selected from groups represented by Formulae CY204 to CY217.

In one or more embodiments, in Formula 201, xa1 may be 1, R₂₀₁ may be a group represented by one selected from Formulae CY201 to CY203, xa2 may be 0, and R₂₀₂ may be a group represented by one selected from Formulae CY204 to CY207.

In one or more embodiments, each of Formulae 201 and 202 may not include a group represented by one selected from Formulae CY201 to CY203.

In one or more embodiments, each of Formulae 201 and 202 may not include a group represented by one selected from Formulae CY201 to CY203, and may include at least one selected from groups represented by Formulae CY204 to CY217.

In one or more embodiments, each of Formulae 201 and 202 may not include a group represented by one selected from Formulae CY201 to CY217.

For example, the hole transport region 120 may include one selected from Compounds HT1 to HT46, m-MTDATA, TDATA, 2-TNATA, NPB(NPD), p-NPB, TPD, Spiro-TPD, Spiro-NPB, methylated NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA) and polyaniline/poly(4-styrenesulfonate) (PANI/PSS), or any combination thereof:

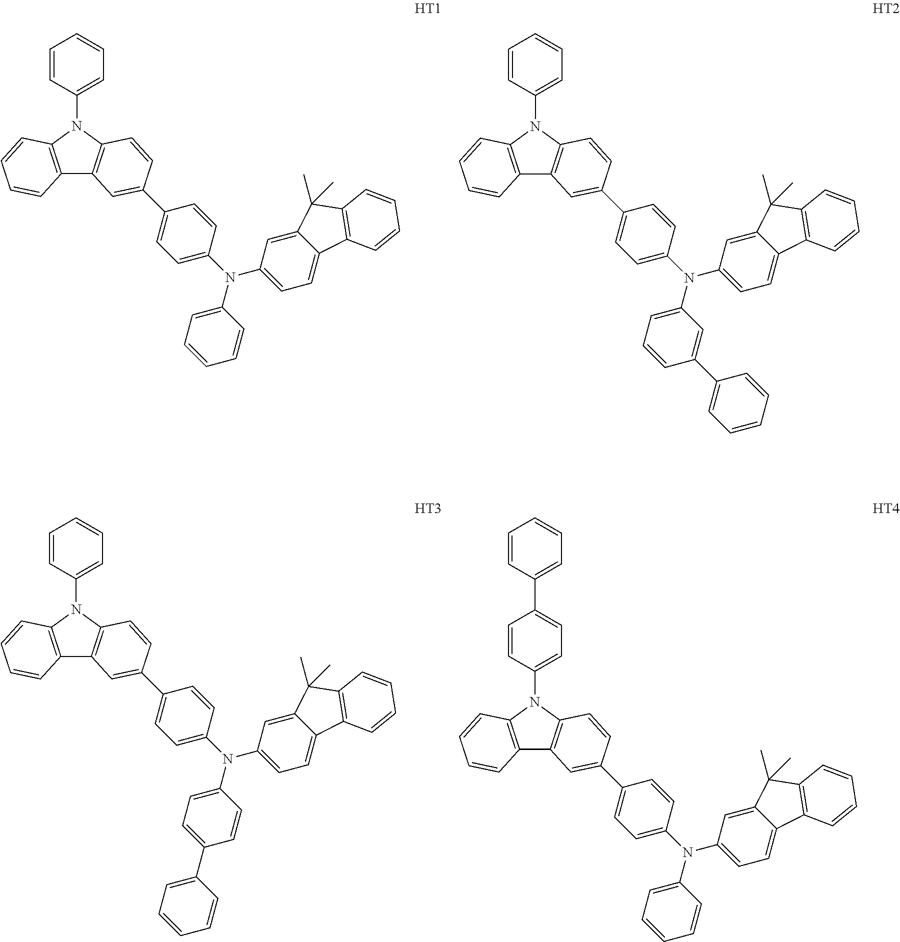 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccccc3)c3ccc(-c4ccc5c(c4)c4ccccc4n5-c4ccc(-c5ccccc5)cc4)cc3)cc21]

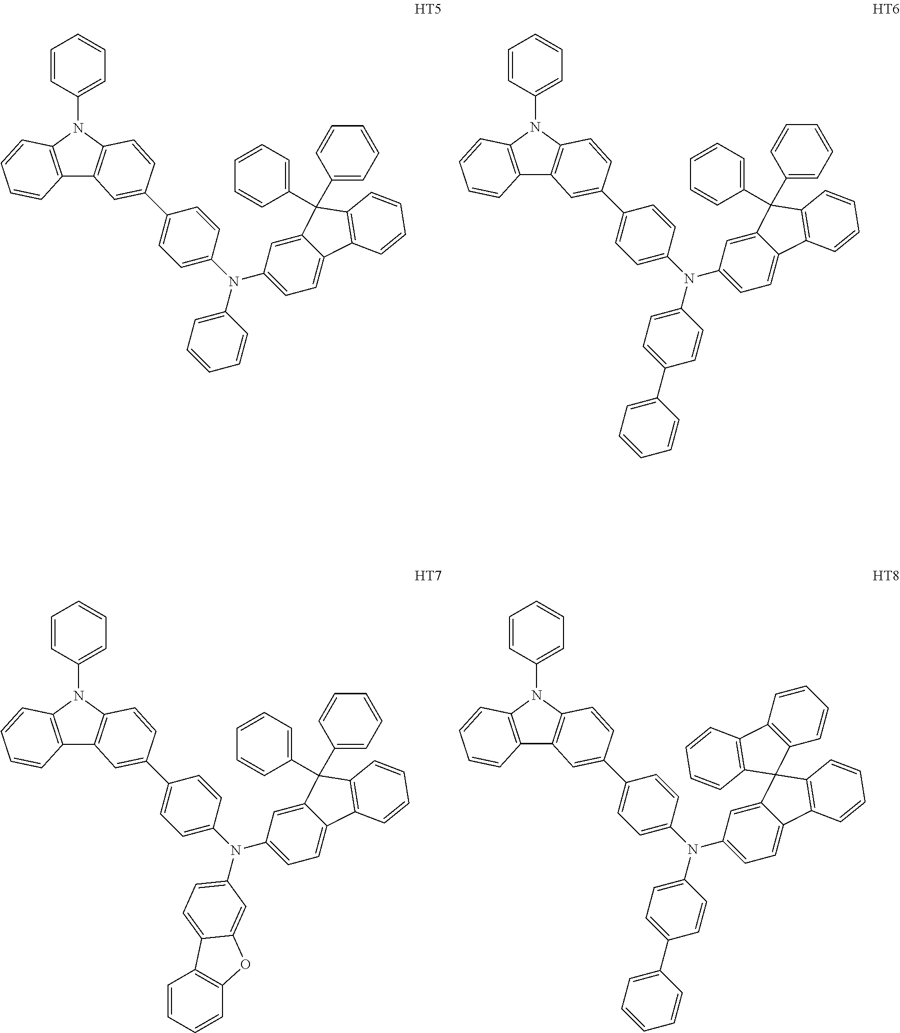 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

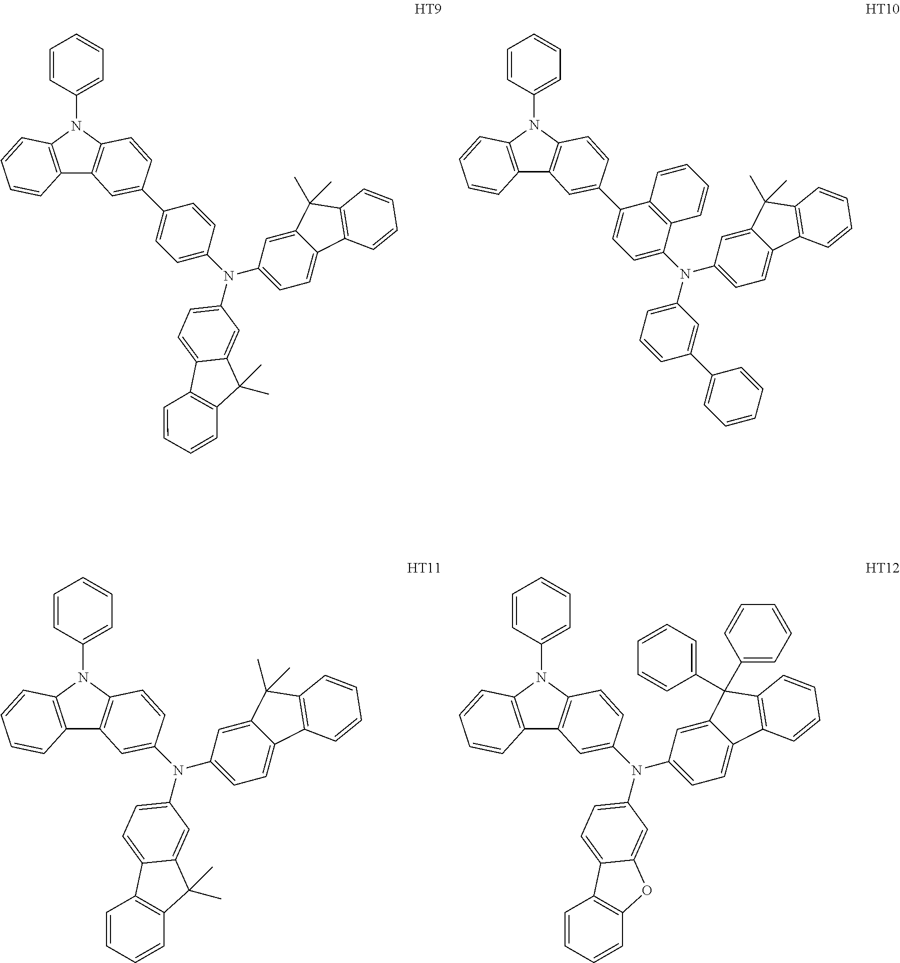 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

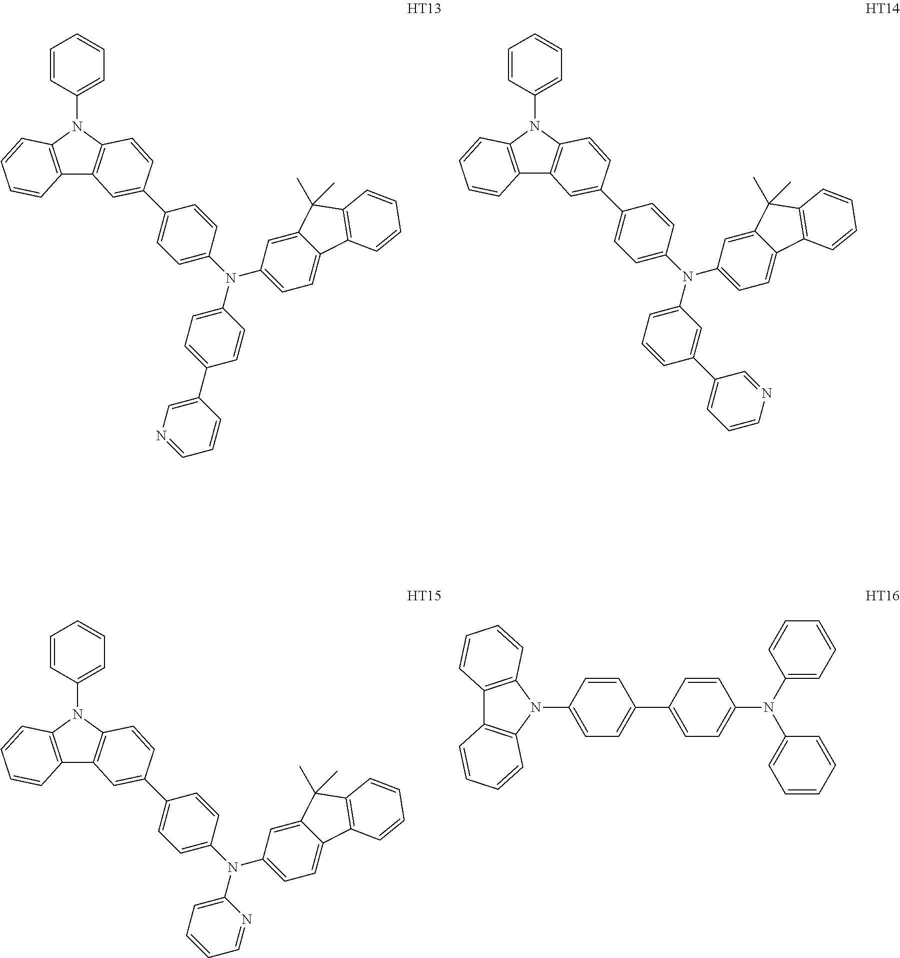 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1]

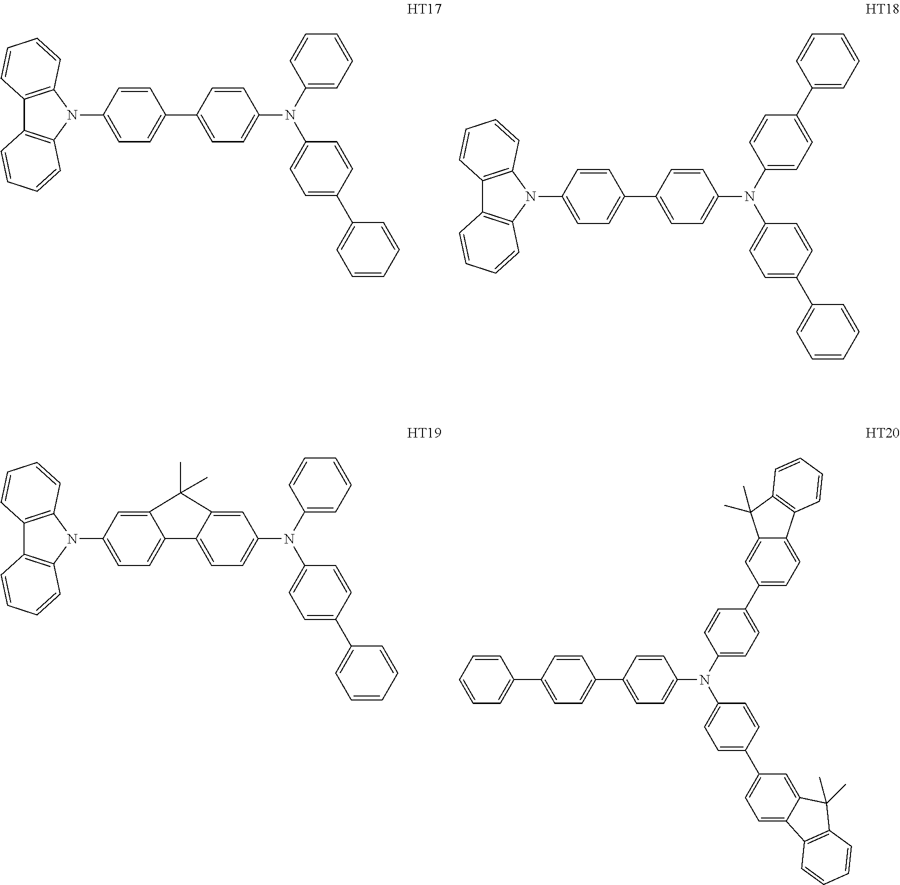 [SMILES: c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21]

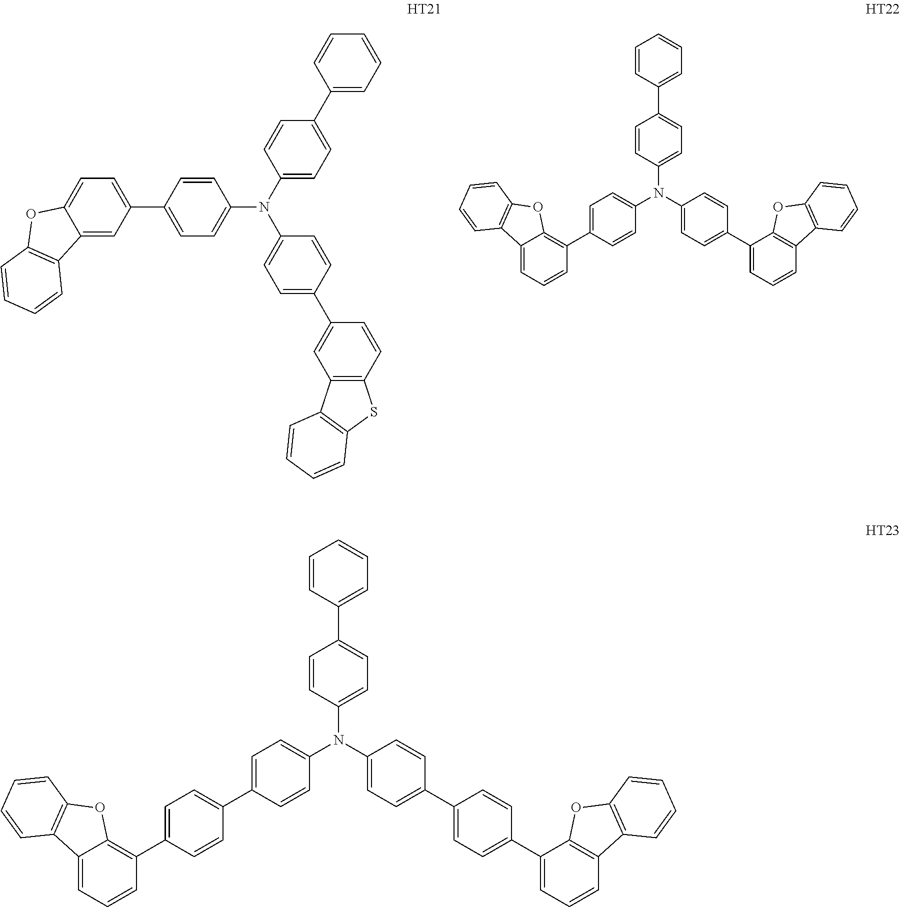 [SMILES: c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

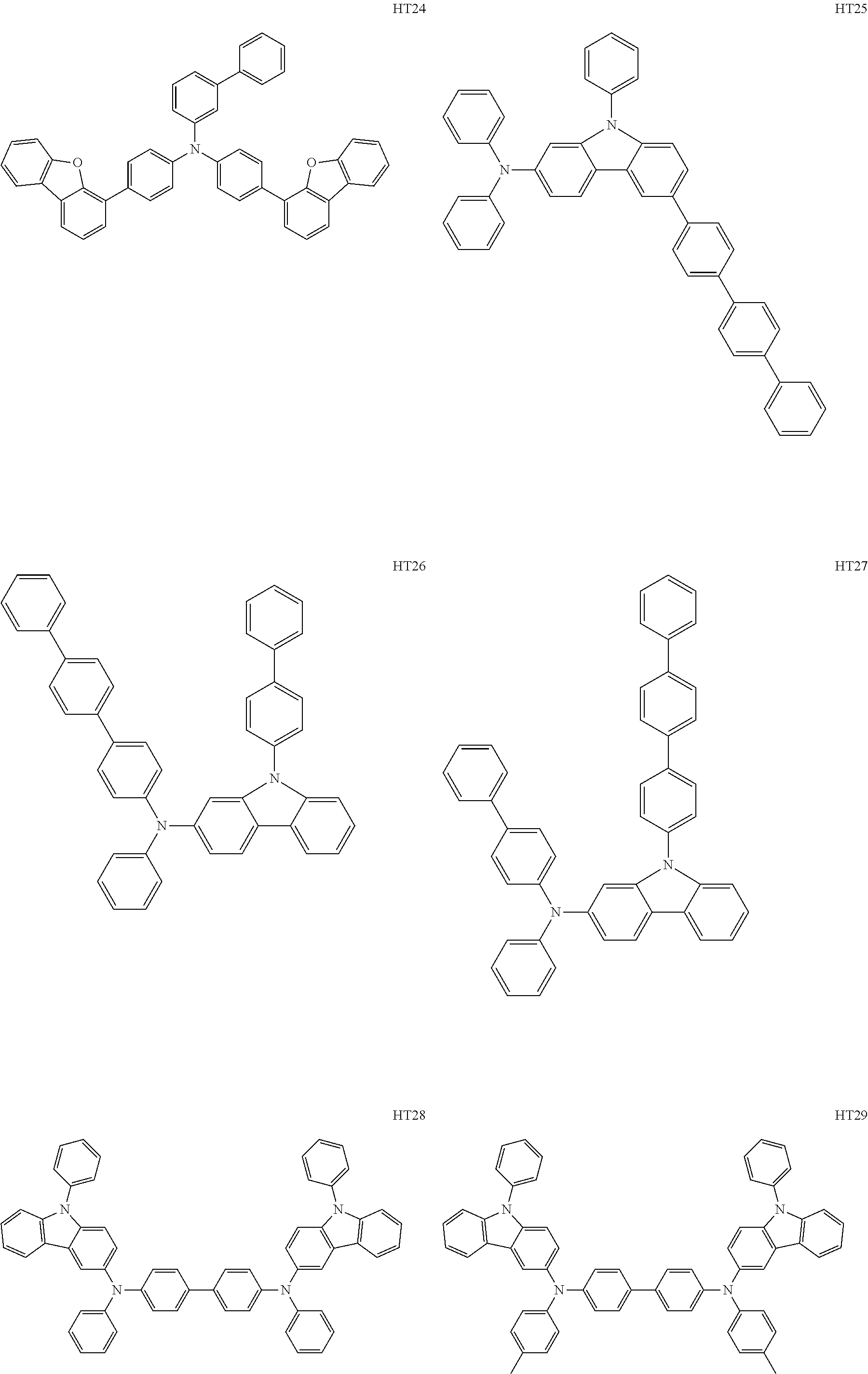

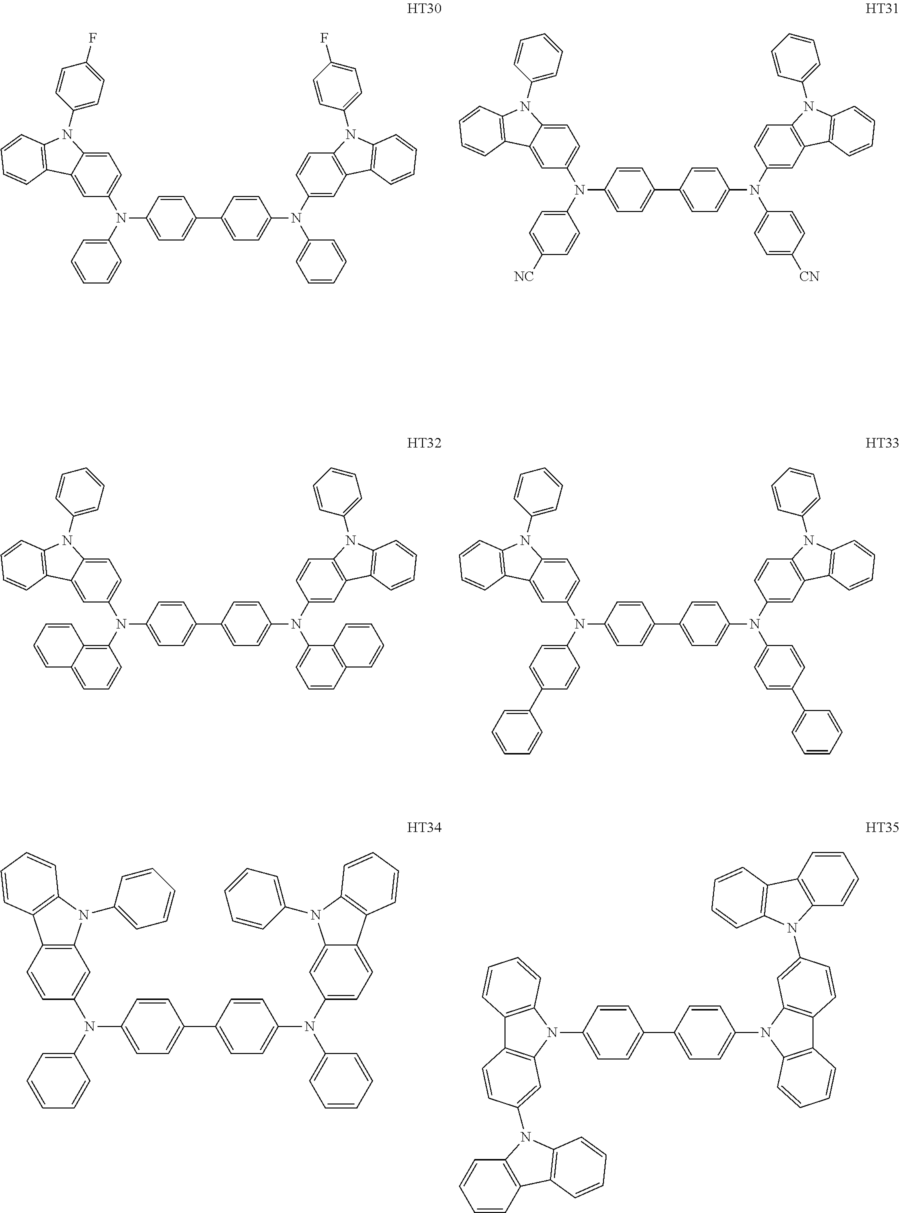

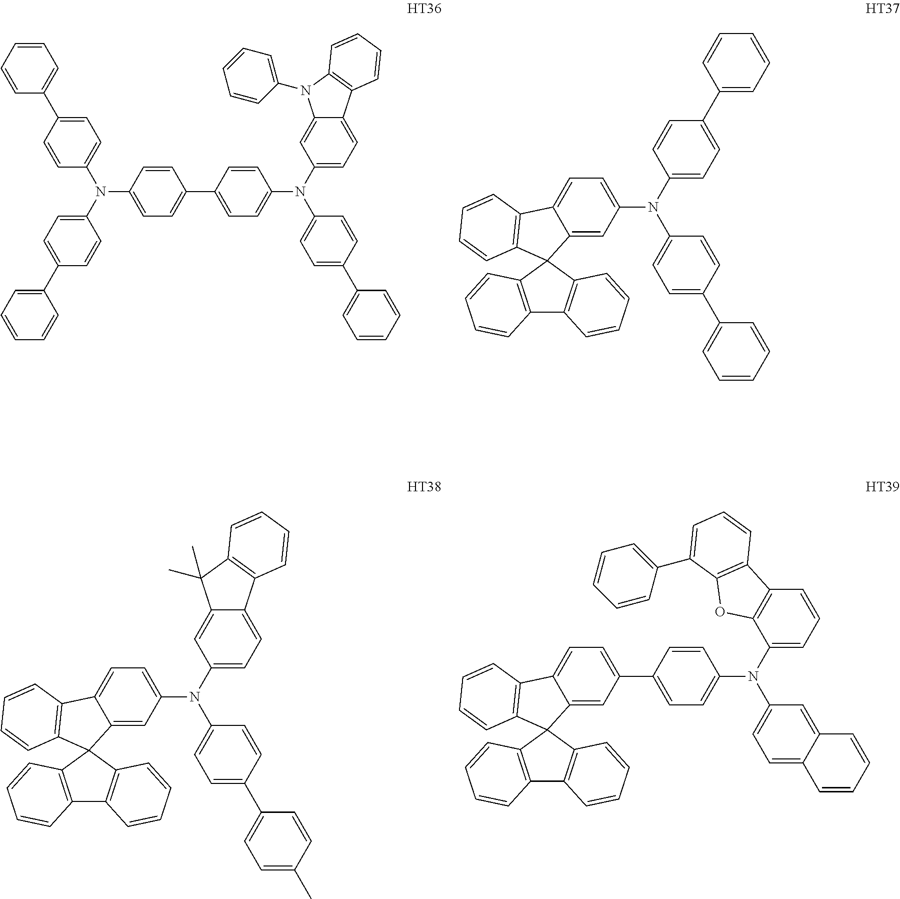 [SMILES: c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1 ; c1ccc(-c2ccc(N(c3ccc(-c4ccccc4)cc3)c3ccc4c(c3)C3(c5ccccc5-c5ccccc53)c3ccccc3-4)cc2)cc1 ; Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; c1ccc(-c2cccc3c2oc2c(N(c4ccc(-c5ccc6c(c5)C5(c7ccccc7-c7ccccc75)c5ccccc5-6)cc4)c4ccc5ccccc5c4)cccc23)cc1]

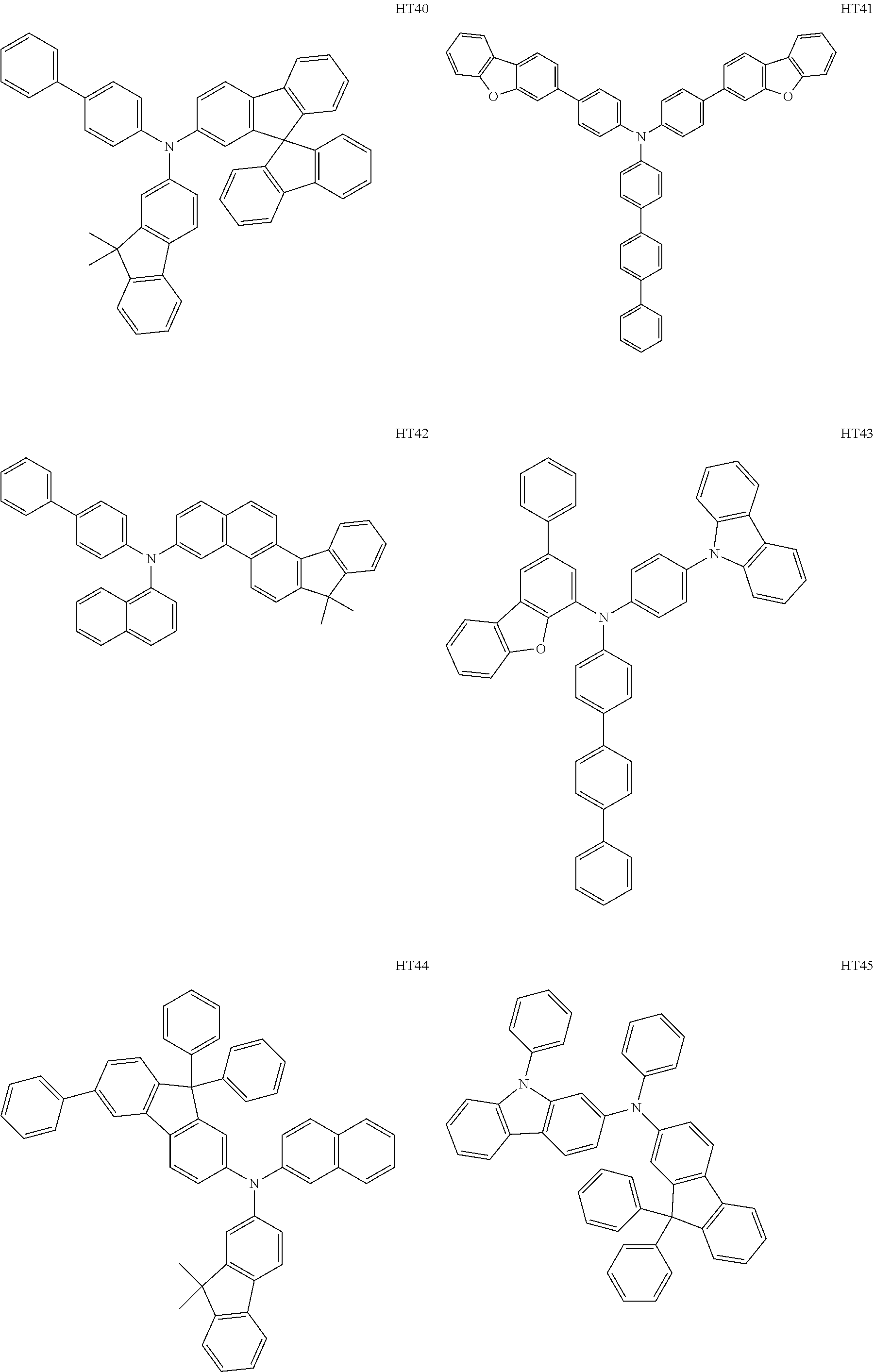

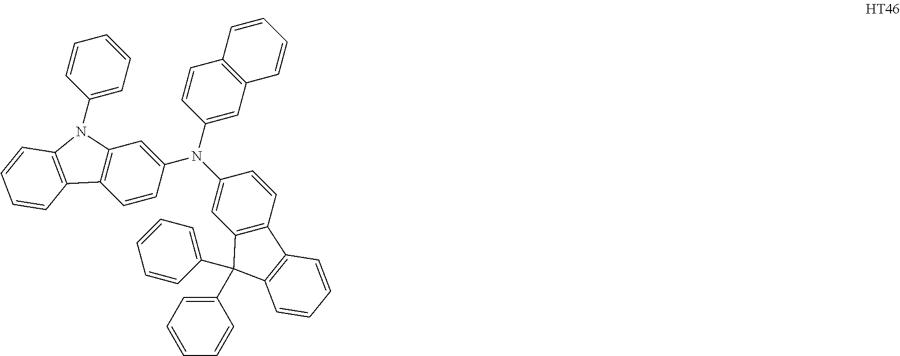 [SMILES: c1ccc(-n2c3ccccc3c3ccc(N(c4ccc5c(c4)C(c4ccccc4)(c4ccccc4)c4ccccc4-5)c4ccc5ccccc5c4)cc32)cc1]

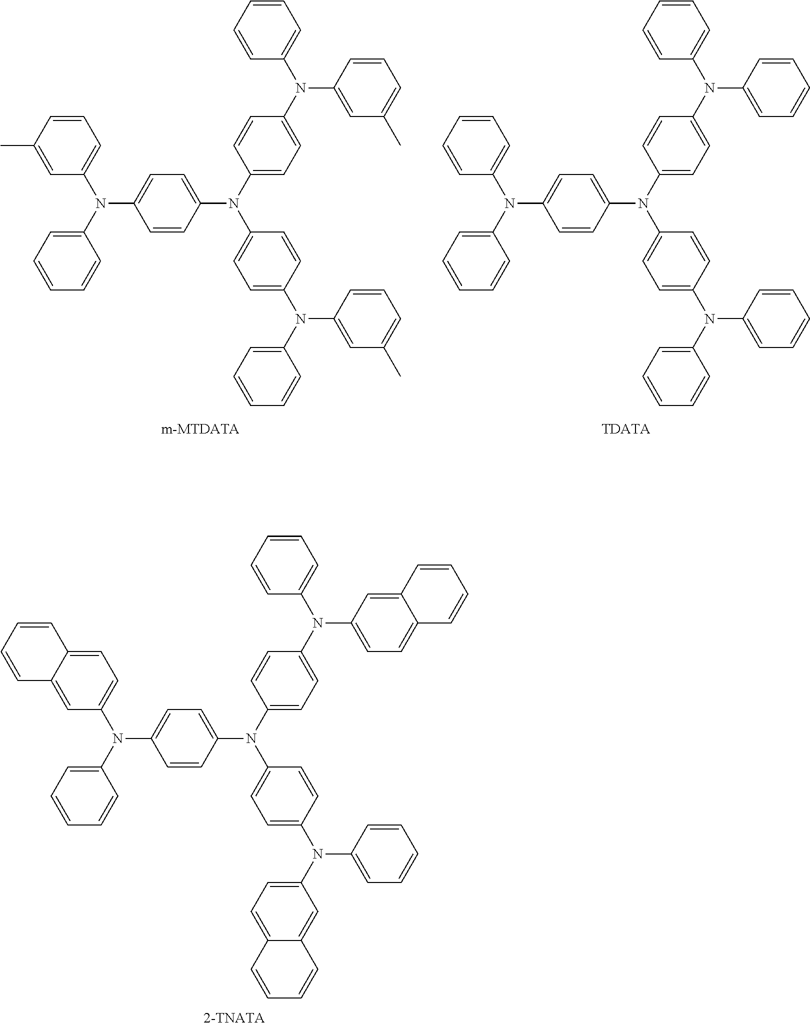

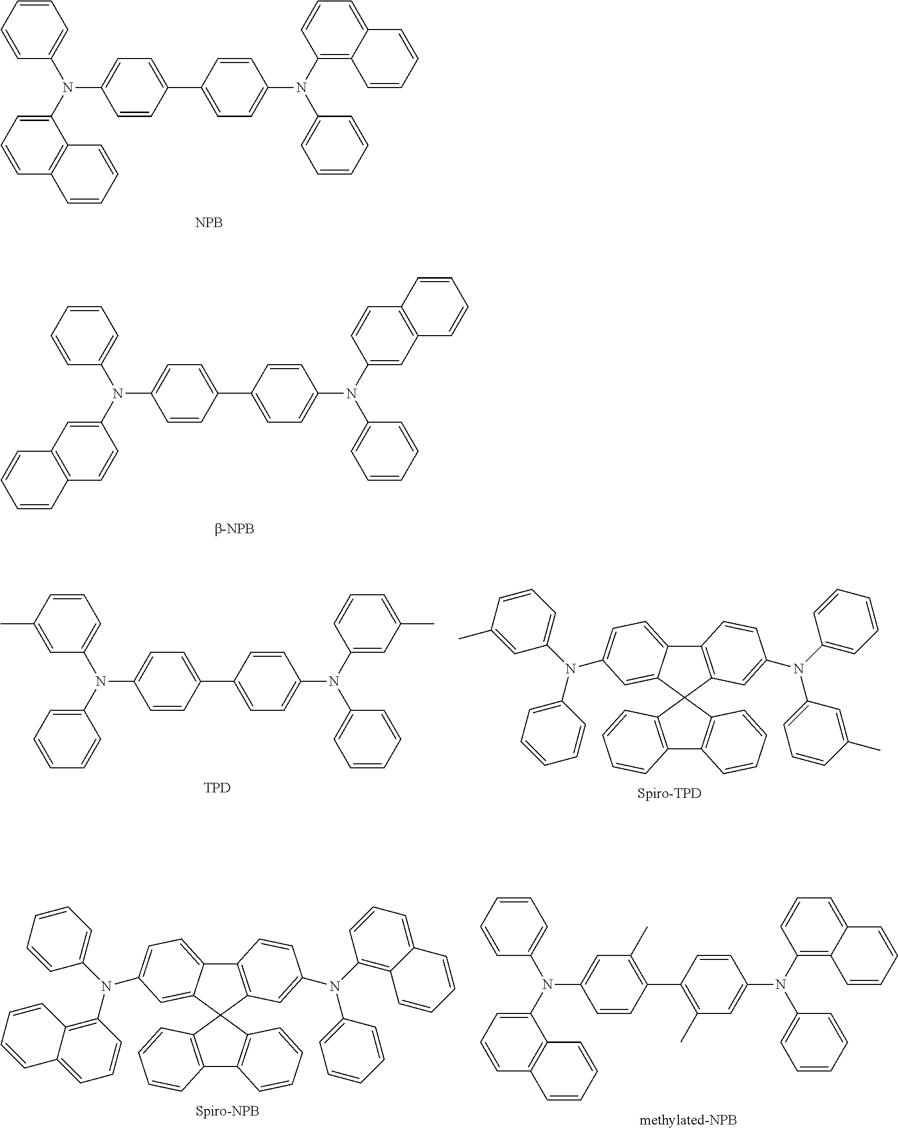

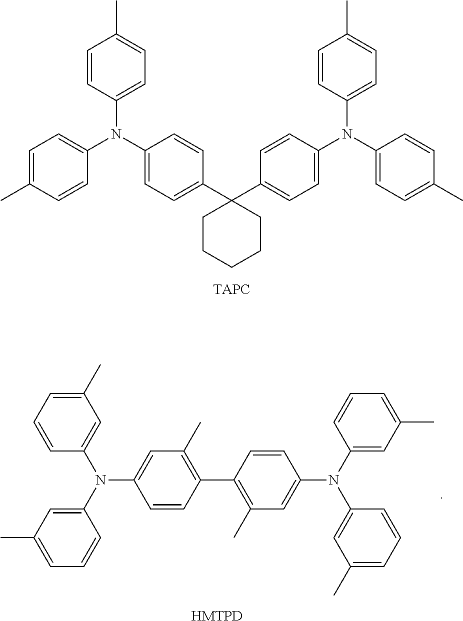

A thickness of the hole transport region 120 may be in a range of about 50 Å to about 10,000 Å, for example, about 100 Å to about 4,000 Å. When the hole transport region 120 includes a hole injection layer, a hole transport layer, or any combination thereof, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region 120, the hole injection layer, and the hole transport layer are within these ranges, suitable or satisfactory hole transporting characteristics may be obtained without a substantial increase in driving voltage.

The emission auxiliary layer may increase light-emission efficiency by compensating for an optical resonance distance according to the wavelength of light emitted by the emission layer 130, and the electron blocking layer may block or reduce the leakage of electrons from the emission layer 130 to the hole transport region 120. Materials that may be included in the hole transport region 120 may be included in the emission auxiliary layer and the electron blocking layer.

p-Dopant

The hole transport region 120 may further include, in addition to the materials described above, a charge-generation material for the improvement of conductive characteristics (e.g., electrically conductive characteristics). The charge-generation material may be uniformly or non-uniformly dispersed in the hole transport region 120 (e.g., in the form of a single layer consisting of a charge-generation material).

The charge-generation material may be, for example, a p-dopant.

For example, the lowest unoccupied molecular orbital (LUMO) energy level of the p-dopant may be −3.5 eV or less.

In one or more embodiments, the p-dopant may include a quinone derivative, a cyano group-containing compound, a compound containing element EL1 and element EL2, or any combination thereof.

Examples of the quinone derivative are TCNQ, F4-TCNQ, and the like.

Examples of the cyano group-containing compound may include HAT-CN, a compound represented by Formula 221, and the like:

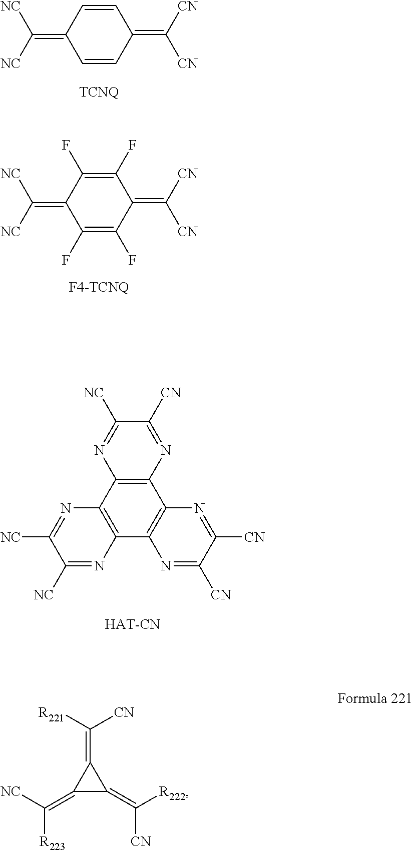

wherein, in Formula 221,

R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and

at least one selected from R₂₂₁ to R₂₂₃ may each independently be a C₃-C₆₀ carbocyclic group or a C₁-C₆₀ heterocyclic group, each substituted with: a cyano group; —F; —Cl; —Br; —I; a C₁-C₂₀ alkyl group substituted with a cyano group, —F, —Cl, —Br, —I, or any combination thereof; or any combination thereof.

In the compound containing element EL1 and element EL2, element EL1 may be a metal, a metalloid, or any combination thereof, and element EL2 may be a non-metal, a metalloid, or any combination thereof.

Examples of the metal may include: an alkali metal (e.g., lithium (Li), sodium (Na), potassium (K), rubidium (Rb), cesium (Cs), etc.); an alkaline earth metal (e.g., beryllium (Be), magnesium (Mg), calcium (Ca), strontium (Sr), barium (Ba), etc.); a transition metal (e.g., titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc.); a post-transition metal (e.g., zinc (Zn), indium (In), tin (Sn), etc.); a lanthanide metal (e.g., lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), etc.); and the like.

Examples of the metalloid may include silicon (Si), antimony (Sb), tellurium (Te), and the like.

Examples of the non-metal may include oxygen (O), halogen (e.g., F, Cl, Br, I, etc.), and the like.

Examples of the compound containing element EL1 and element EL2 may include a metal oxide, a metal halide (e.g., a metal fluoride, a metal chloride, a metal bromide, a metal iodide, etc.), a metalloid halide (e.g., a metalloid fluoride, a metalloid chloride, a metalloid bromide, a metalloid iodide, etc.), a metal telluride, or any combination thereof.

Examples of the metal oxide may include a tungsten oxide (e.g., WO, W₂O₃, WO₂, WO₃, W₂O₅, etc.), a vanadium oxide (e.g., VO, V₂O₃, VO₂, V₂O₅, etc.), a molybdenum oxide (e.g., MoO, Mo₂O₃, MoO₂, MoO₃, Mo₂O₅, etc.), a rhenium oxide (e.g., ReO₃, etc.), and the like.

Examples of the metal halide may include an alkali metal halide, an alkaline earth metal halide, a transition metal halide, a post-transition metal halide, a lanthanide metal halide, and the like.

Examples of the alkali metal halide may include LiF, NaF, KF, RbF, CsF, LiCl, NaCl, KCl, RbCl, CsCl, LiBr, NaBr, KBr, RbBr, CsBr, LiI, NaI, KI, RbI, CsI, and the like.

Examples of the alkaline earth metal halide may include BeF₂, MgF₂, CaF₂, SrF₂, BaF₂, BeCl₂, MgCl₂, CaCl₂), SrCl₂, BaCl₂, BeBr₂, MgBr₂, CaBr₂, SrBr₂, BaBr₂, BeI₂, MgI₂, CaI₂, SrI₂, BaI₂, and the like.

Examples of the transition metal halide may include a titanium halide (e.g., TiF₄, TiCl₄, TiBr₄, TiI₄, etc.), a zirconium halide (e.g., ZrF₄, ZrCl₄, ZrBr₄, ZrI₄, etc.), a hafnium halide (e.g., HfF₄, HfCl₄, HfBr₄, Hfl₄, etc.), a vanadium halide (e.g., VF₃, VCl₃, VBr₃, VI₃, etc.), a niobium halide (e.g., NbF₃, NbCl₃, NbBr₃, NbI₃, etc.), a tantalum halide (e.g., TaF₃, TaCl₃, TaBr₃, TaI₃, etc.), a chromium halide (e.g., CrF₃, CrO₃, CrBr₃, CrI₃, etc.), a molybdenum halide (e.g., MoF₃, MoCl₃, MoBr₃, MoI₃, etc.), a tungsten halide (e.g., WF₃, WCl₃, WBr₃, WI₃, etc.), a manganese halide (e.g., MnF₂, MnCl₂, MnBr₂, MnI₂, etc.), a technetium halide (e.g., TcF₂, TcCl₂, TcBr₂, TcI₂, etc.), a rhenium halide (e.g., ReF₂, ReCl₂, ReBr₂, ReI₂, etc.), a ferrous halide (e.g., FeF₂, FeCl₂, FeBr₂, FeI₂, etc.), a ruthenium halide (e.g., RuF₂, RuCl₂, RuBr₂, RuI₂, etc.), an osmium halide (e.g., OsF₂, OsCl₂, OsBr₂, OsI₂, etc.), a cobalt halide (e.g., CoF₂, COCl₂, CoBr₂, CoI₂, etc.), a rhodium halide (e.g., RhF₂, RhCl₂, RhBr₂, RhI₂, etc.), an iridium halide (e.g., IrF₂, IrCl₂, IrBr₂, IrI₂, etc.), a nickel halide (e.g., NiF₂, NiCl₂, NiBr₂, NiI₂, etc.), a palladium halide (e.g., PdF₂, PdCl₂, PdBr₂, PdI₂, etc.), a platinum halide (e.g., PtF₂, PtCl₂, PtBr₂, PtI₂, etc.), a cuprous halide (e.g., CuF, CuCl, CuBr, CuI, etc.), a silver halide (e.g., AgF, AgCl, AgBr, AgI, etc.), a gold halide (e.g., AuF, AuCl, AuBr, AuI, etc.), and the like.

Examples of the post-transition metal halide may include a zinc halide (e.g., ZnF₂, ZnCl₂, ZnBr₂, ZnI₂, etc.), an indium halide (e.g., InI₃, etc.), a tin halide (e.g., SnI₂, etc.), and the like.

Examples of the lanthanide metal halide may include YbF, YbF₂, YbF₃, SmF₃, YbCl, YbCl₂, YbCl₃, SmCl₃, YbBr, YbBr₂, YbBr₃, SmBr₃, YbI, YbI₂, YbI₃, SmI₃, and the like.

Examples of the metalloid halide may include an antimony halide (e.g., SbCl₅, etc.) and the like.

Examples of the metal telluride may include an alkali metal telluride (e.g., Li₂Te, Na₂Te, K₂Te, Rb₂Te, Cs₂Te, etc.), an alkaline earth metal telluride (e.g., BeTe, MgTe, CaTe, SrTe, BaTe, etc.), a transition metal telluride (e.g., TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, etc.), a post-transition metal telluride (e.g., ZnTe, etc.), a lanthanide metal telluride (e.g., LaTe, CeTe, PrTe, NdTe, PmTe, EuTe, GdTe, TbTe, DyTe, HoTe, ErTe, TmTe, YbTe, LuTe, etc.), and the like.

### Emission Layer 130

The light-emitting device 10 may include the emission layer 130 arranged on the hole transport region 120.

The emission layer 130 may further include, in addition to various suitable organic materials, a metal-containing compound, such as an organometallic compound, an inorganic material, such as a quantum dot, and/or the like.

In one or more embodiments, the emission layer 130 may include i) two or more emitting units sequentially stacked between the first electrode 110 and the second electrode 150, and ii) a charge generation layer arranged between the two or more emitting units. When the emission layer 130 includes the emitting units and the charge generation layer as described above, the light-emitting device 10 may be a tandem light-emitting device.

When the light-emitting device 10 is a full-color light-emitting device, the emission layer 130 may be patterned into a red emission layer, a green emission layer, and/or a blue emission layer, according to a sub-pixel. In one or more embodiments, the emission layer 130 may have a stacked structure of two or more layers of a red emission layer, a green emission layer, and a blue emission layer, in which the two or more layers contact each other or are separated from each other to emit white light. In one or more embodiments, the emission layer 130 may include two or more materials of a red light-emitting material, a green light-emitting material, and a blue light-emitting material, in which the two or more materials are mixed together with each other in a single layer to emit white light.

The emission layer 130 may include a host and a dopant. The dopant may include a phosphorescent dopant, a fluorescent dopant, or any combination thereof.

An amount of the dopant in the emission layer 130 may be in a range of about 0.01 part by weight to about 15 parts by weight based on 100 parts by weight of the host.

In one or more embodiments, the emission layer 130 may include a quantum dot.

In one or more embodiments, the emission layer 130 may include a delayed fluorescence material. The delayed fluorescence material may act as a host or a dopant in the emission layer 130.

A thickness of the emission layer 130 may be in a range of about 100 Å to about 1,000 Å, for example, about 200 Å to about 600 Å. When the thickness of the emission layer 130 is within these ranges, excellent luminescence characteristics may be obtained without a substantial increase in driving voltage.

### Host

The host may include a compound represented by Formula 301:

[Ar₃₀₁]_{xb11}-[(L₃₀₁)_{xb1}-R₃₀₁]_{xb21}, Formula 301

wherein, in Formula 301,

Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xb11 may be 1, 2, or 3,

xb1 may be an integer from 0 to 5,

R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),

xb21 may be an integer from 1 to 5, and

Q₃₀₁ to Q₃₀₃ may each be as defined herein in connection with Q₁.

For example, when xb11 in Formula 301 is 2 or more, two or more of Ar₃₀₁(s) may be linked to each other via a single bond.

In one or more embodiments, the host may include a compound represented by Formula 301-1, a compound represented by Formula 301-2, or any combination thereof:

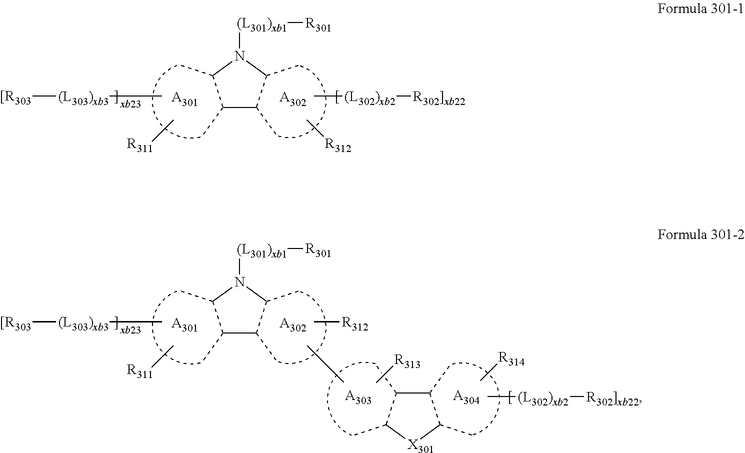

wherein, in Formulae 301-1 and 301-2,

ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

X₃₀₁ may be O, S, N[(L₃₀₄)_{xb4}-R₃₀₄], C(R₃₀₄)(R₃₀₅), or Si(R₃₀₄)(R₃₀₅),

xb22 and xb23 may each independently be 0, 1, or 2,

L₃₀₁, xb1, and R₃₀₁ may each be as defined herein,

L₃₀₂ to L₃₀₄ may each independently be as defined herein in connection with L₃₀₁,

xb2 to xb4 may each independently be as defined herein in connection with xb1, and
R₃₀₂ to R₃₀₅ and R₃₁₁ to R₃₁₄ may each be as defined herein in connection with R₃₀₁.

In one or more embodiments, the host may include an alkali earth metal complex, a post-transition metal complex, or any combination thereof. For example, the host may include a Be complex (e.g., Compound H55), a Mg complex, a Zn complex, or any combination thereof.

In one or more embodiments, the host may include one selected from Compounds H1 to H128, 9,10-di(2-naphthyl)anthracene (ADN), 2-methyl-9,10-bis(naphthalen-2-yl)anthracene (MADN), 9,10-di(2-naphthyl)-2-t-butylanthracene (TBADN), 4,4′-bis(N-carbazolyl)-1,1′-biphenyl (CBP), 1,3-di(carbazol-9-yl)benzene (mCP) and 1,3,5-tri(carbazol-9-yl)benzene (TCP), or any combination thereof:

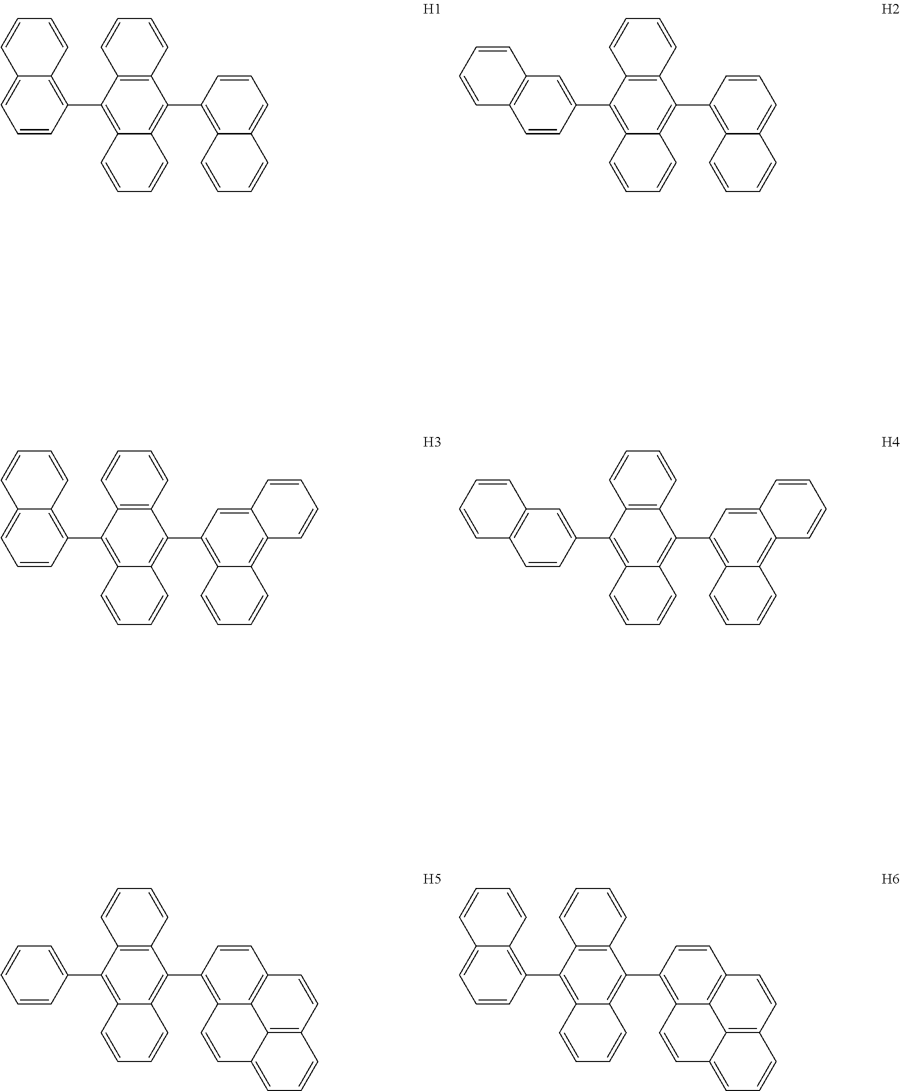

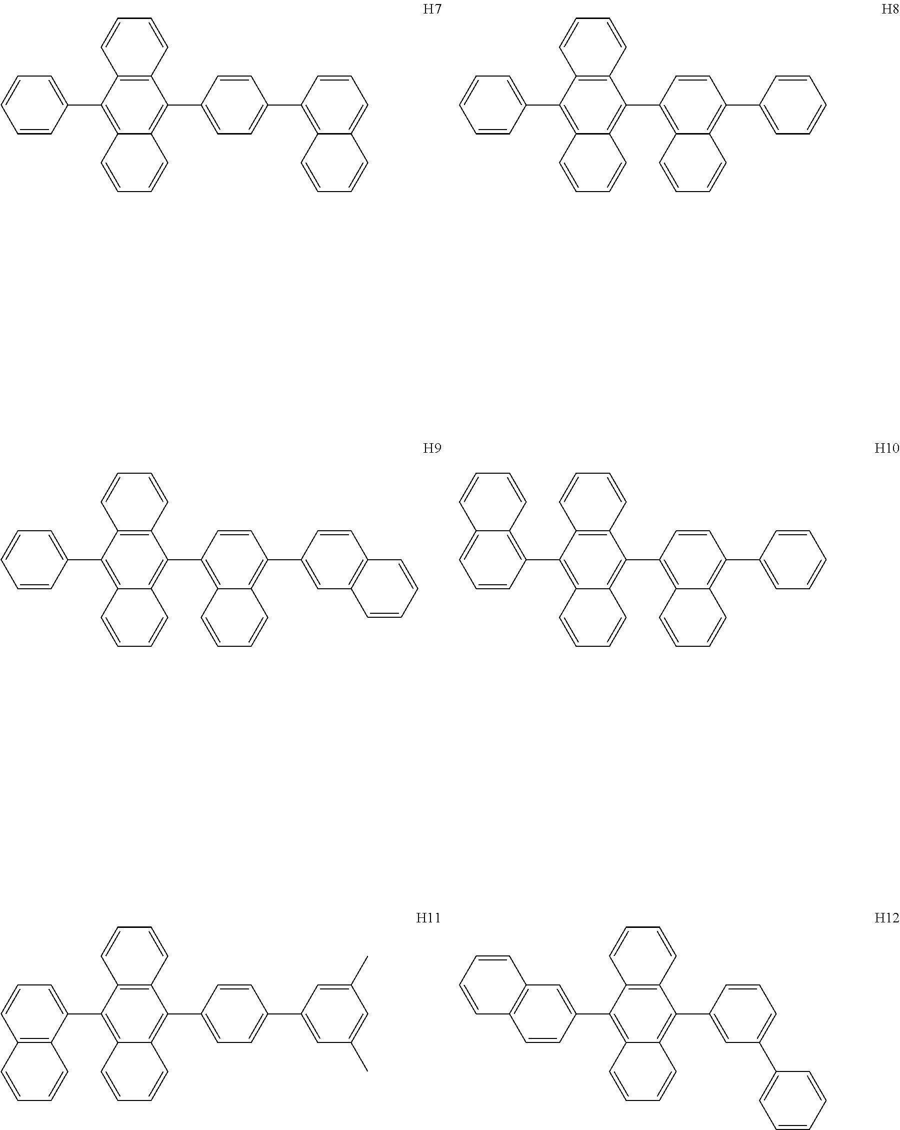

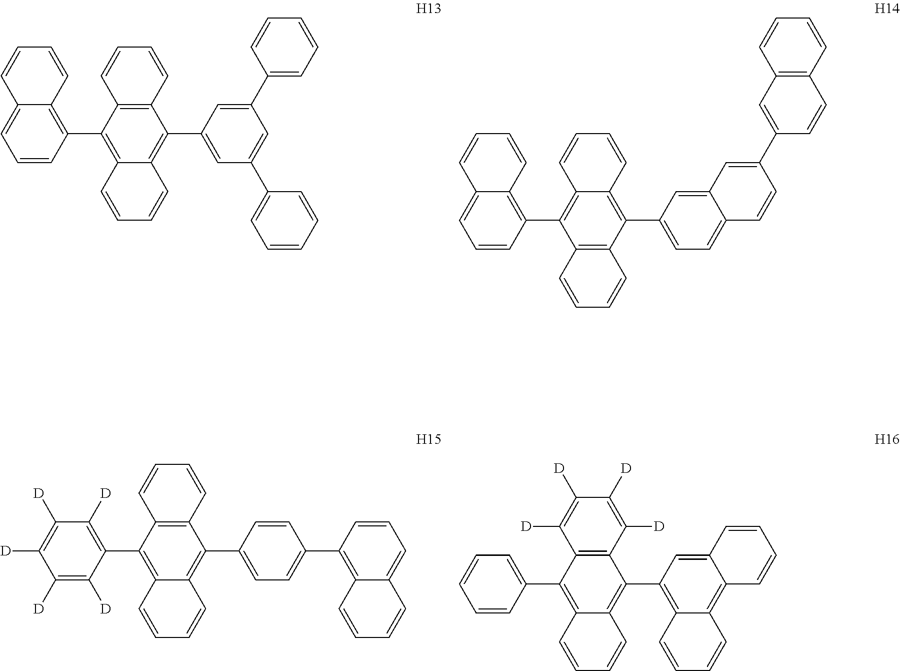

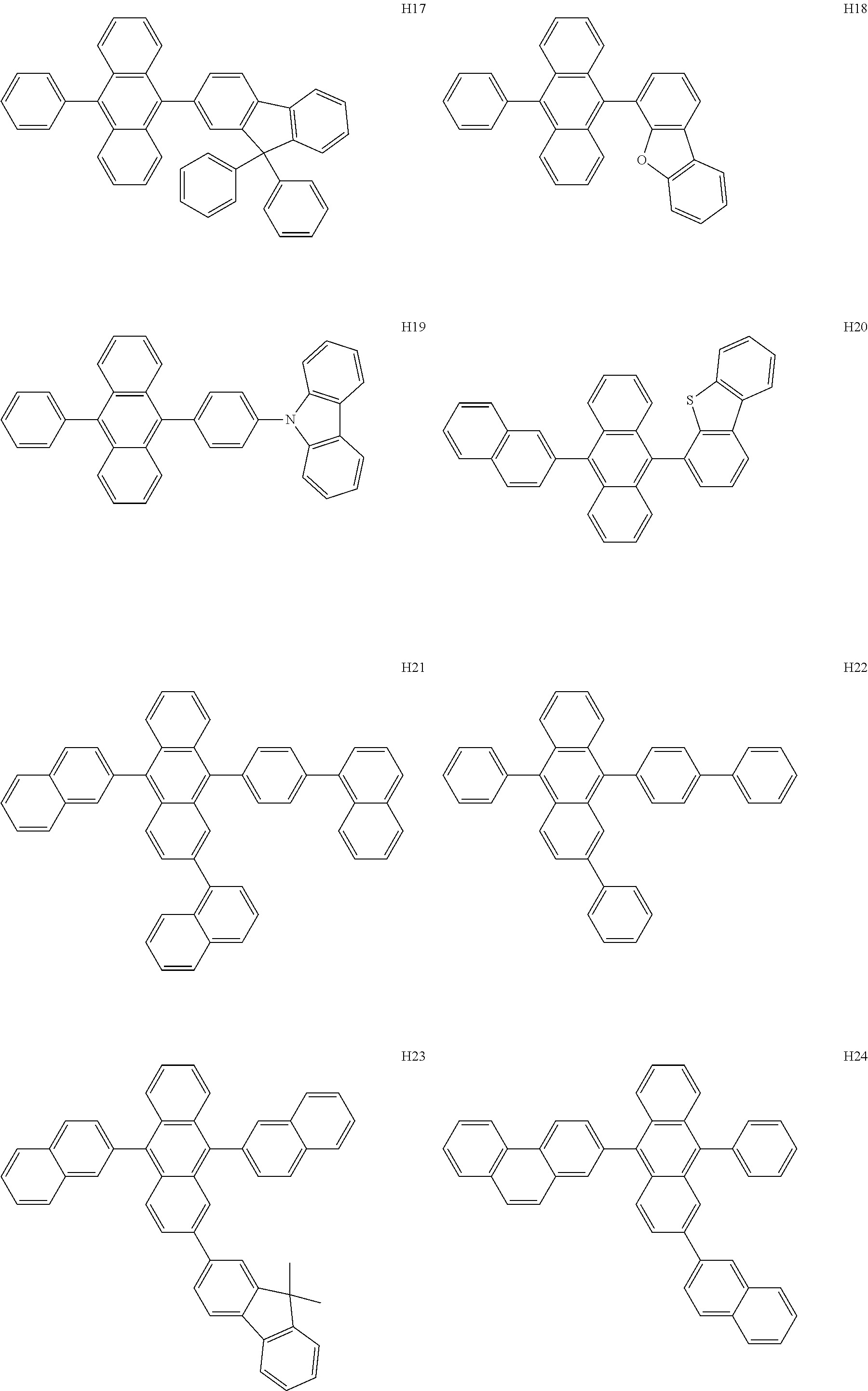

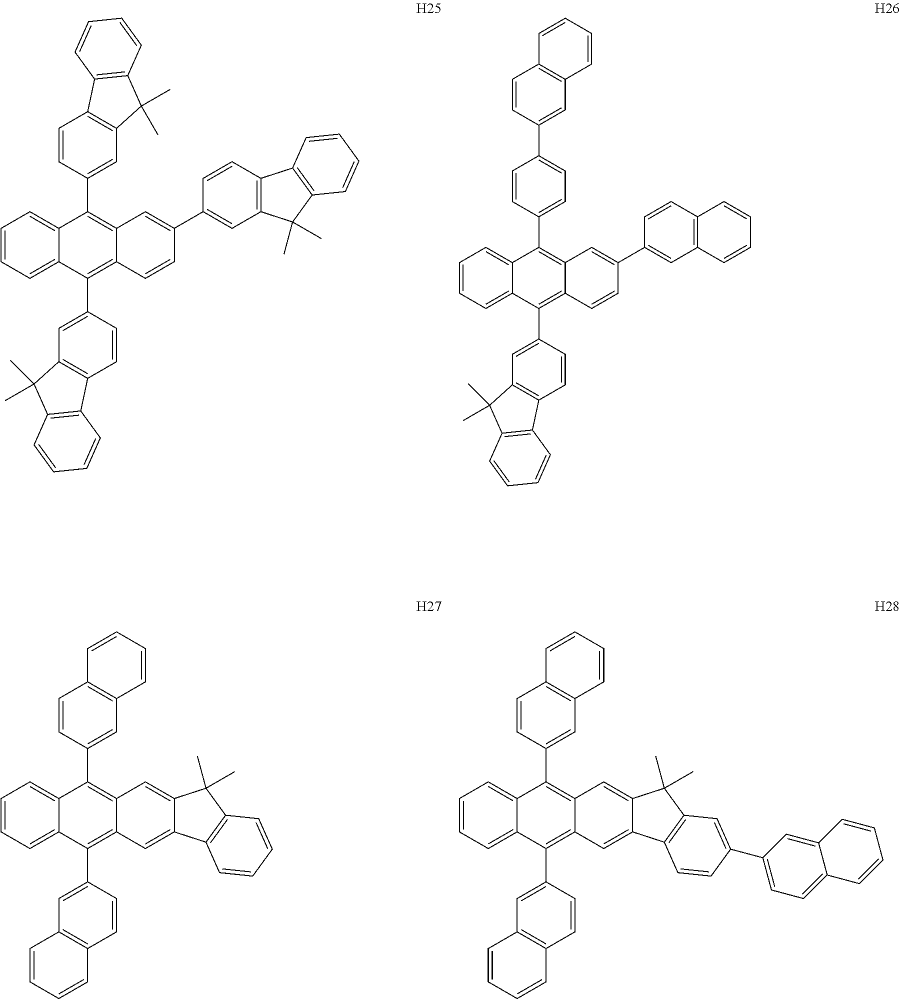

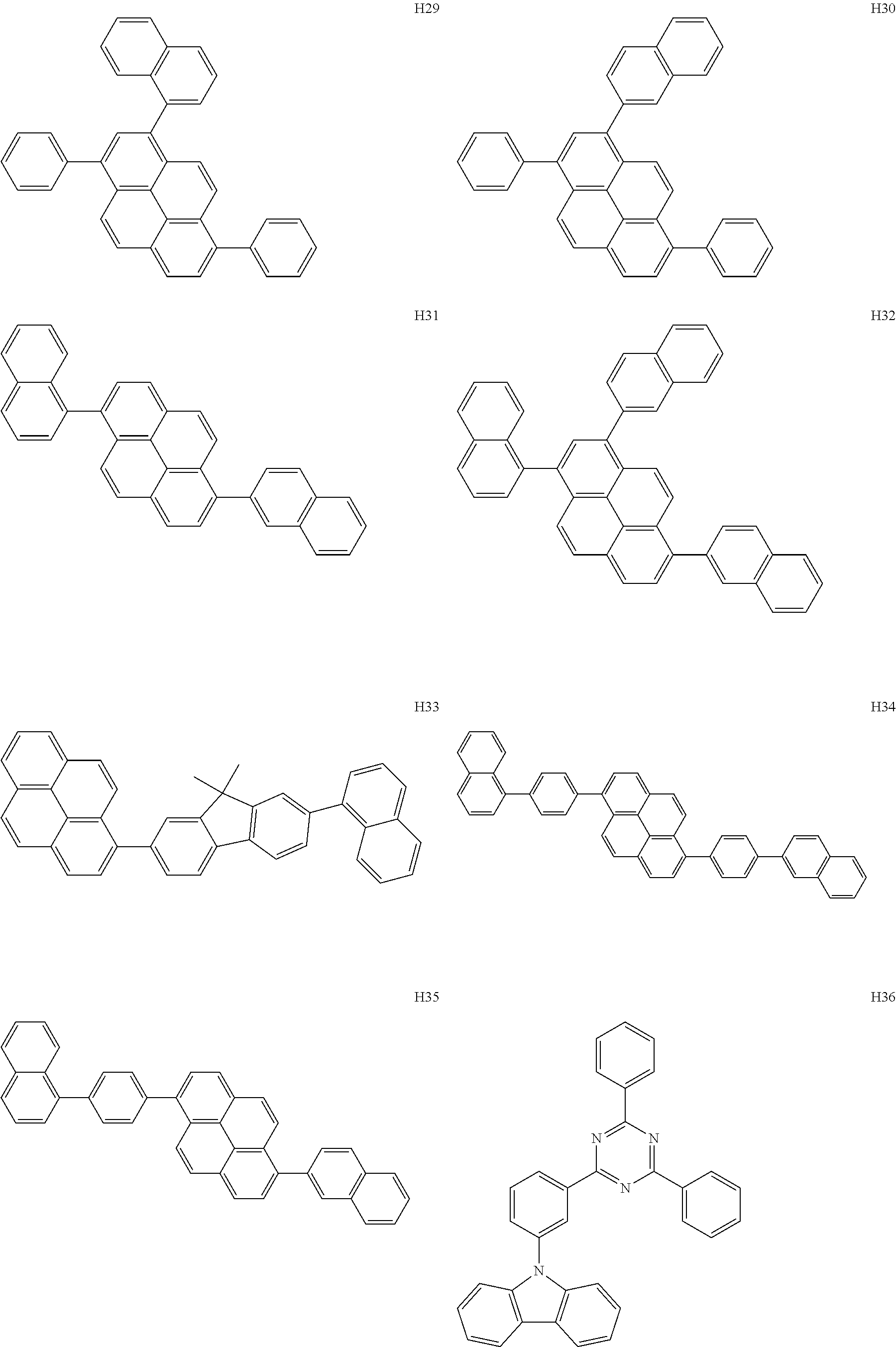

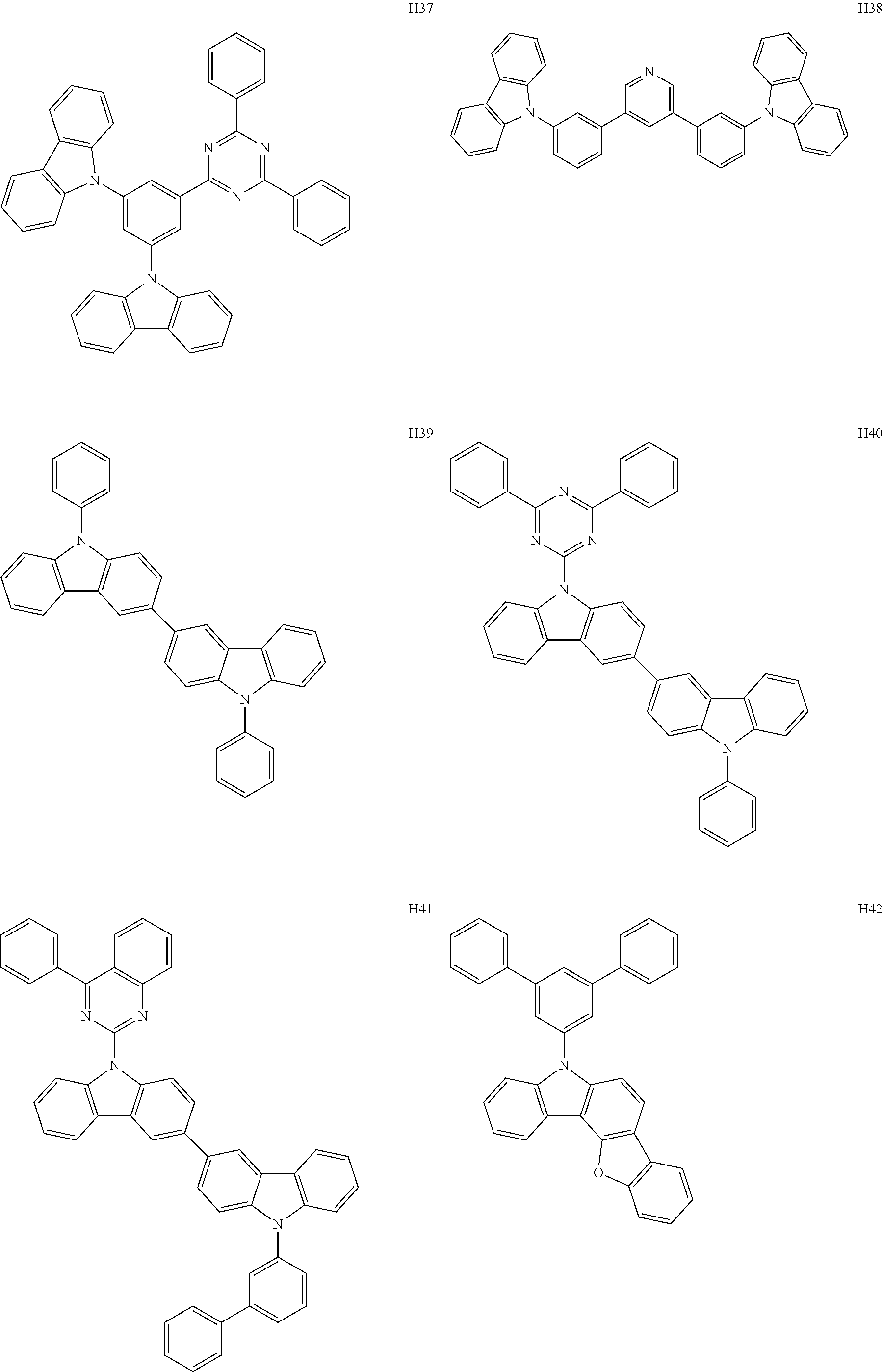 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-n2c3ccccc3c3cc(-c4ccc5c(c4)c4ccccc4n5-c4ccccc4)ccc32)cc1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2cccc(-n3c4ccccc4c4cc(-c5ccc6c(c5)c5ccccc5n6-c5nc(-c6ccccc6)c6ccccc6n5)ccc43)c2)cc1 ; c1ccc(-c2cc(-c3ccccc3)cc(-n3c4ccccc4c4c5oc6ccccc6c5ccc43)c2)cc1]

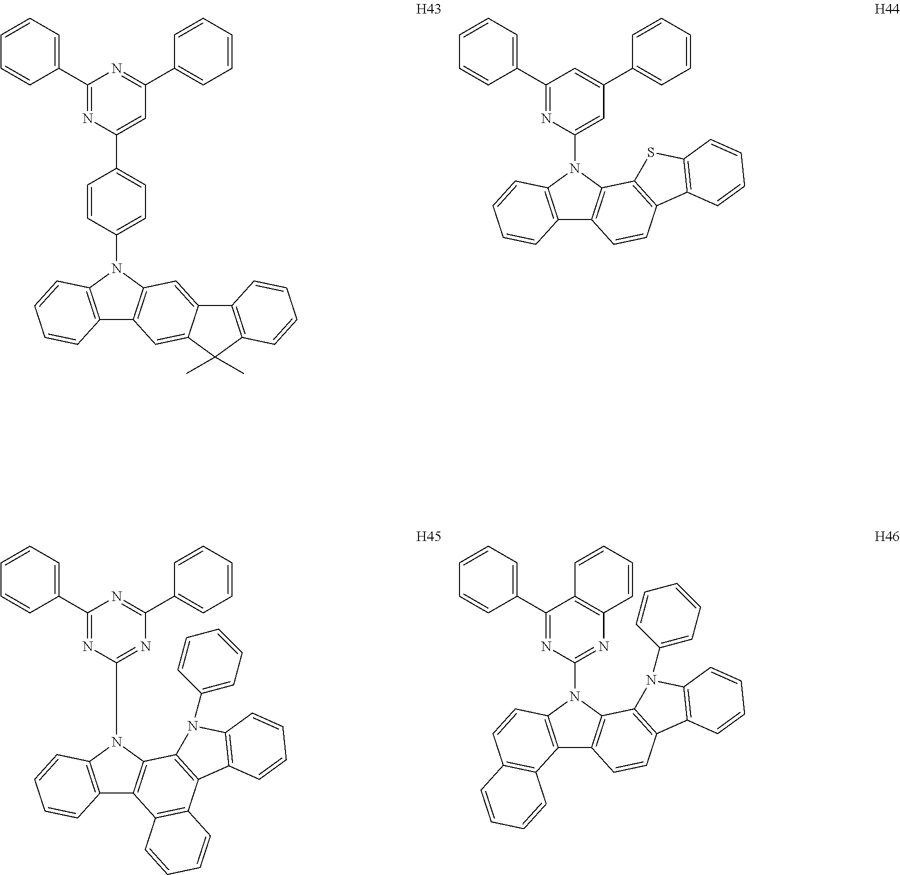 [SMILES: CC1(C)c2ccccc2-c2cc3c(cc21)c1ccccc1n3-c1ccc(-c2cc(-c3ccccc3)nc(-c3ccccc3)n2)cc1 ; c1ccc(-c2cc(-c3ccccc3)nc(-n3c4ccccc4c4ccc5c6ccccc6sc5c43)c2)cc1 ; c1ccc(-c2nc(-c3ccccc3)nc(-n3c4ccccc4c4c5ccccc5c5c6ccccc6n(-c6ccccc6)c5c43)n2)cc1 ; c1ccc(-c2nc(-n3c4ccc5ccccc5c4c4ccc5c6ccccc6n(-c6ccccc6)c5c43)nc3ccccc23)cc1]

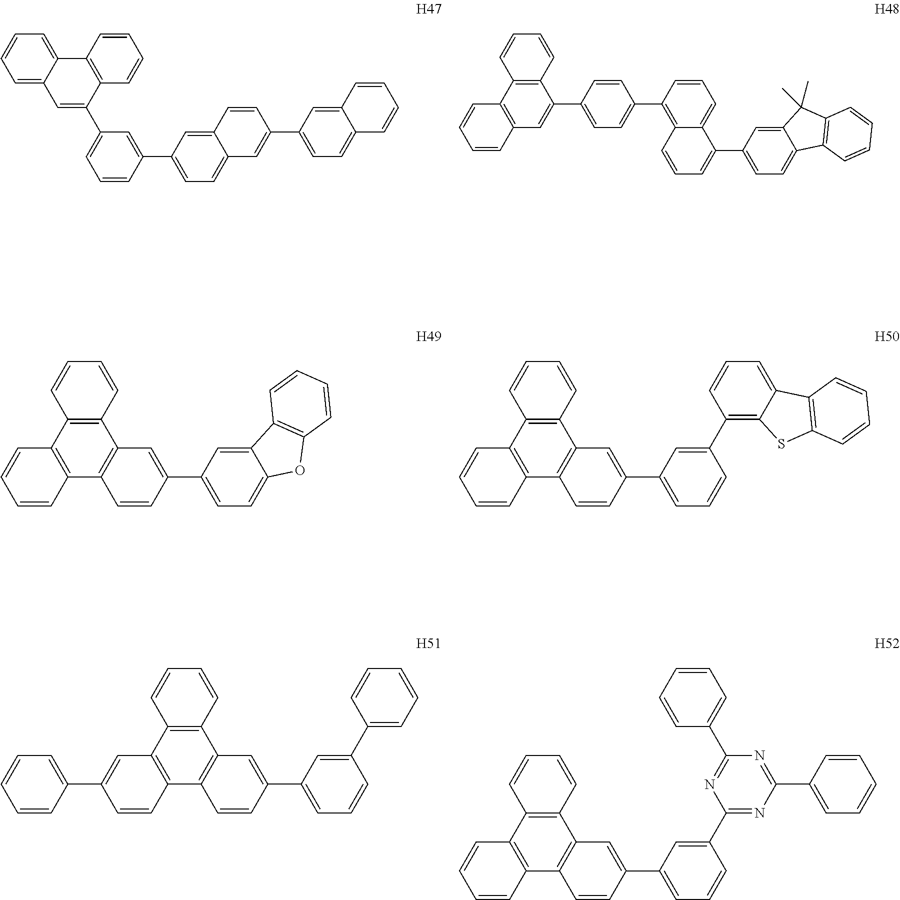

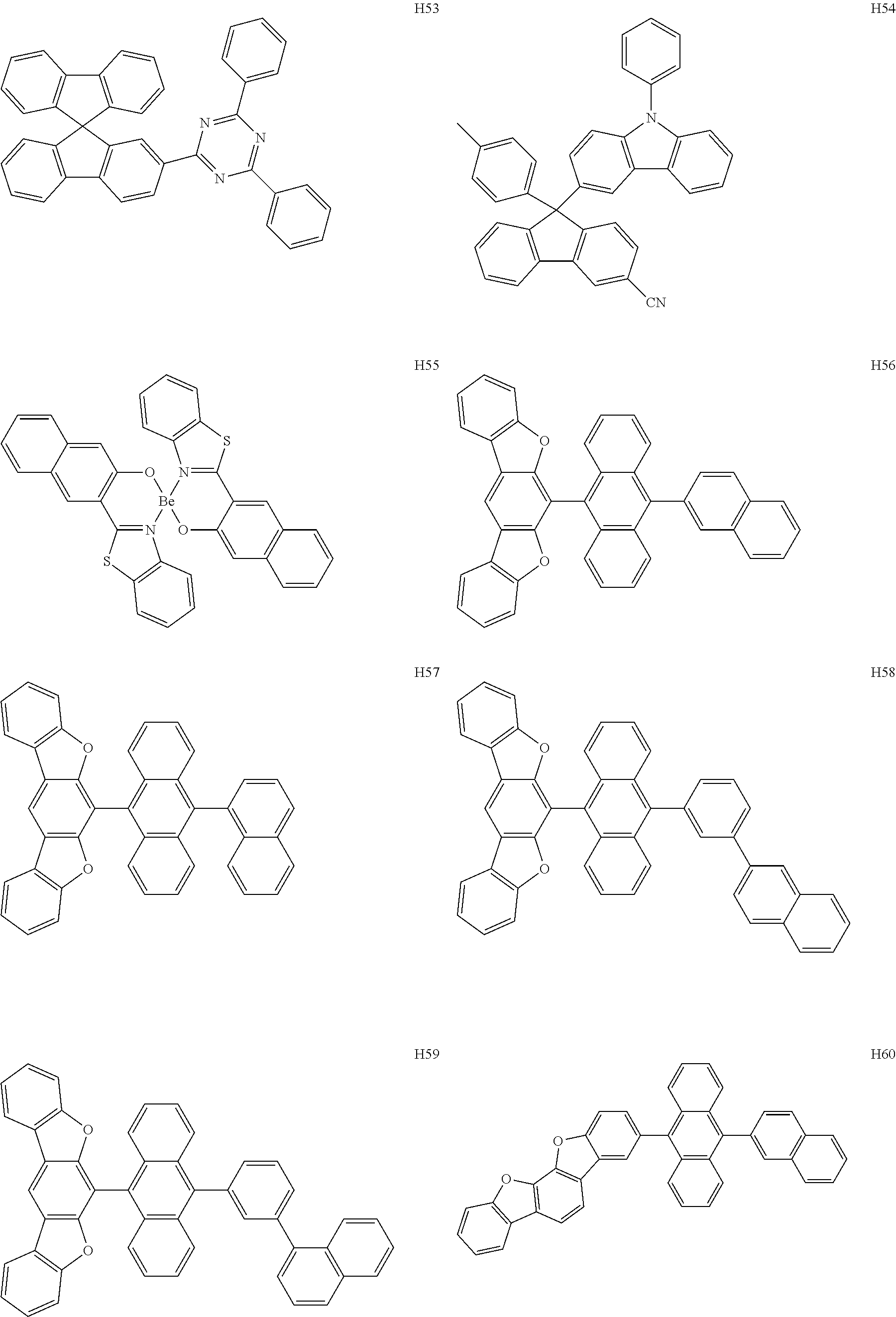

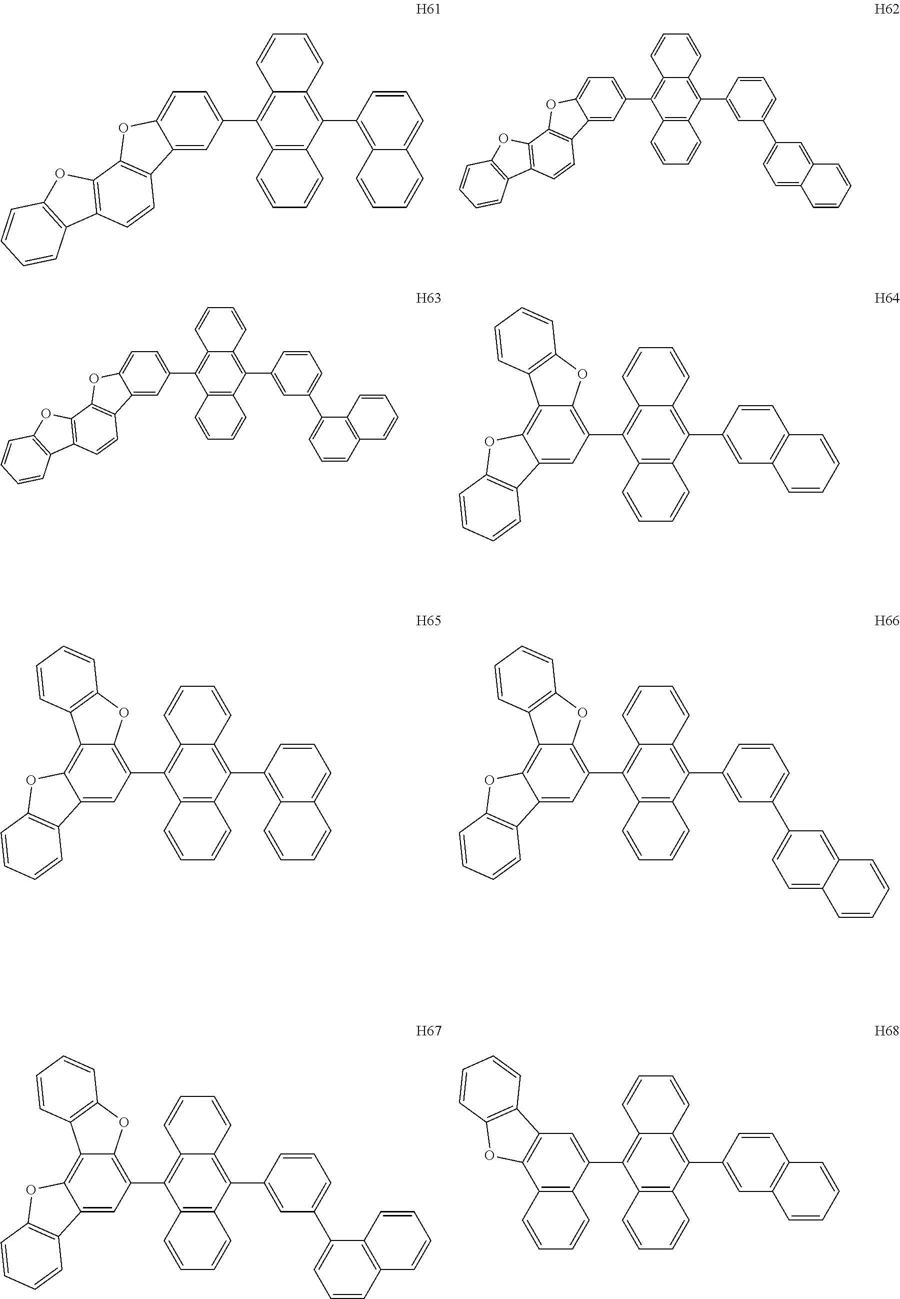

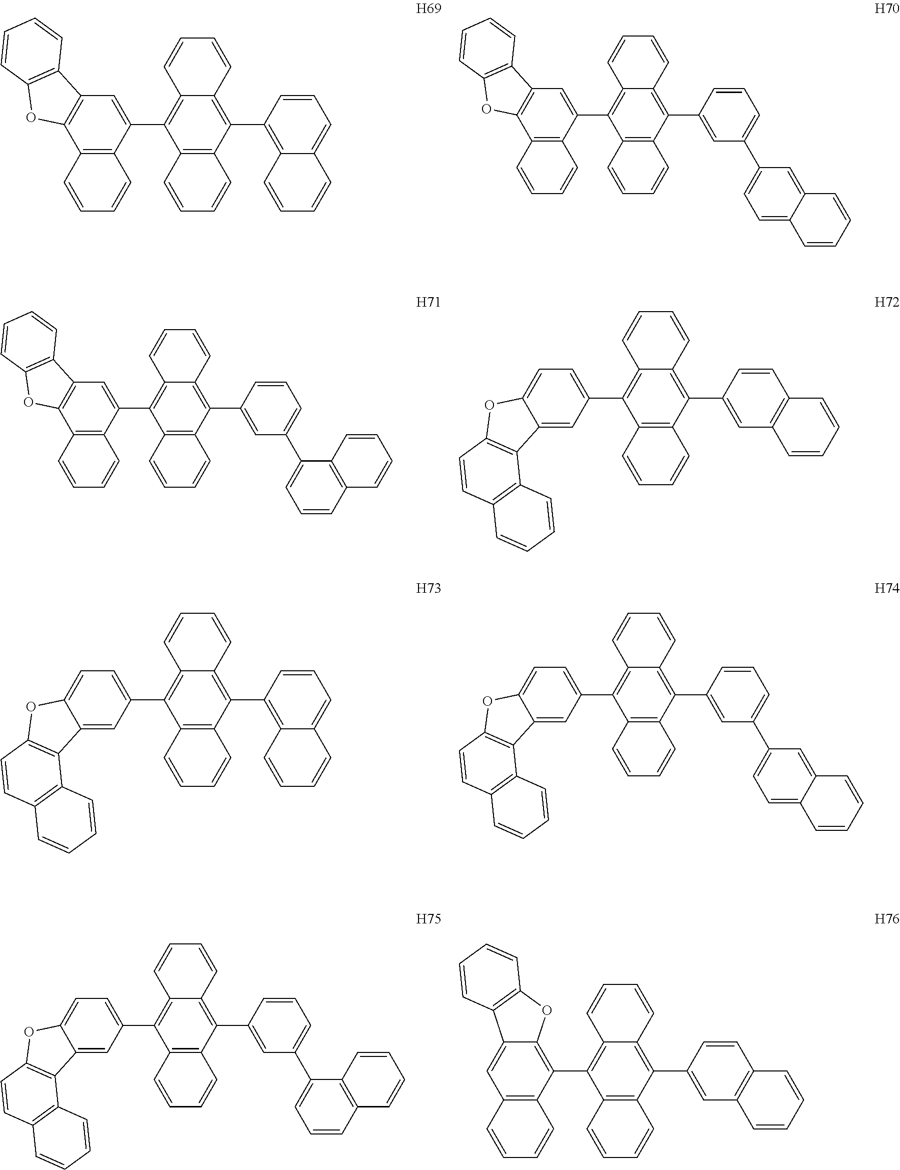

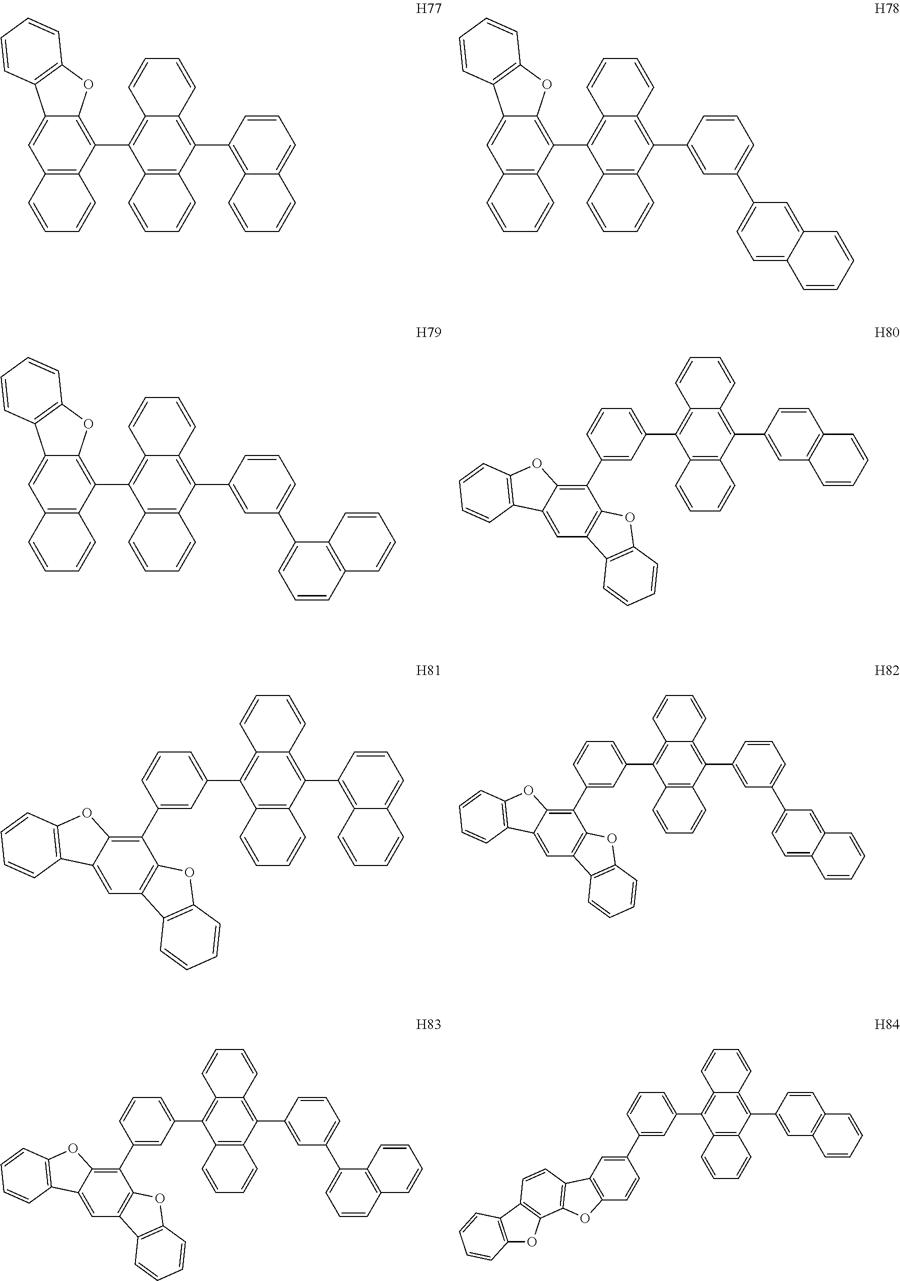

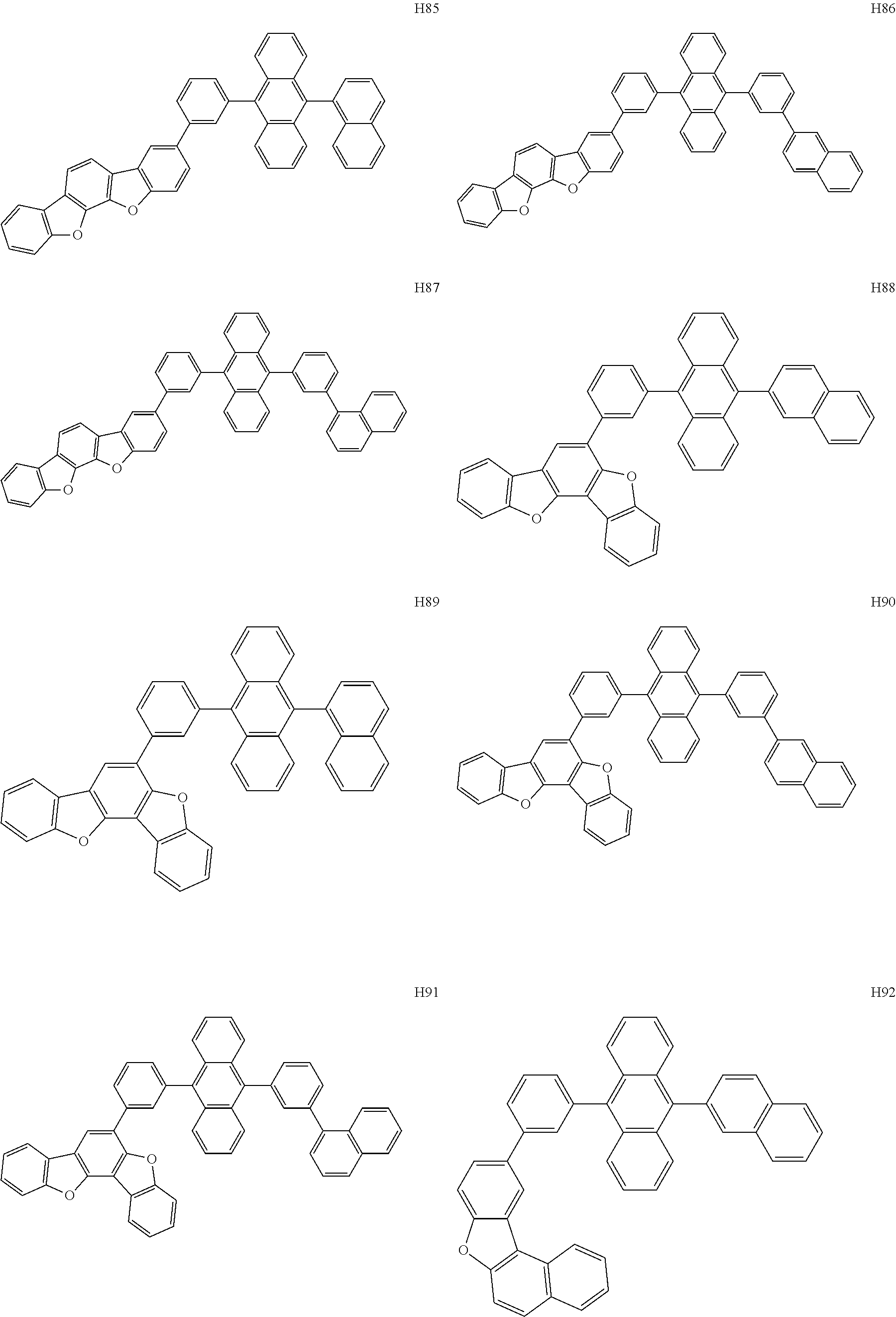

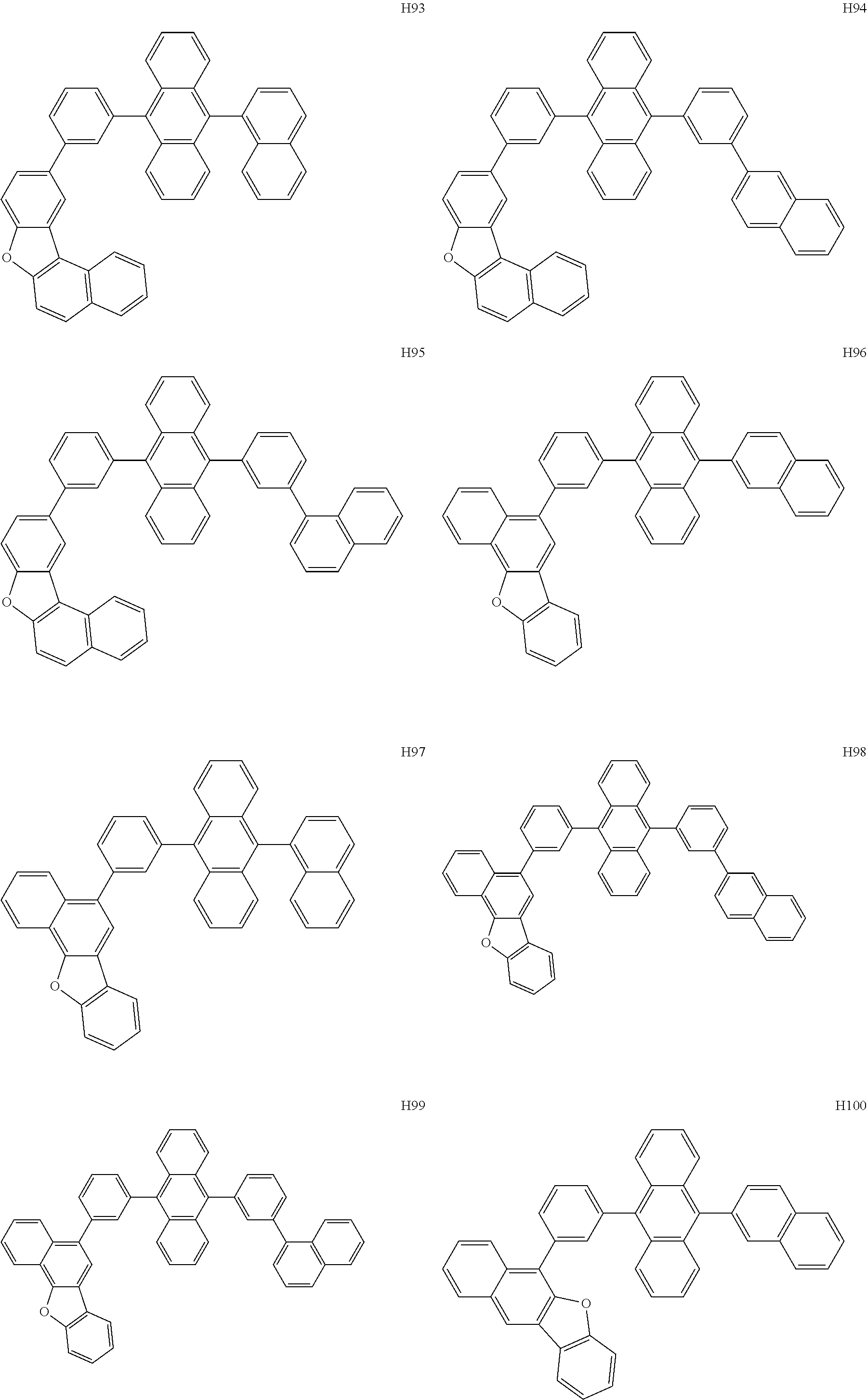

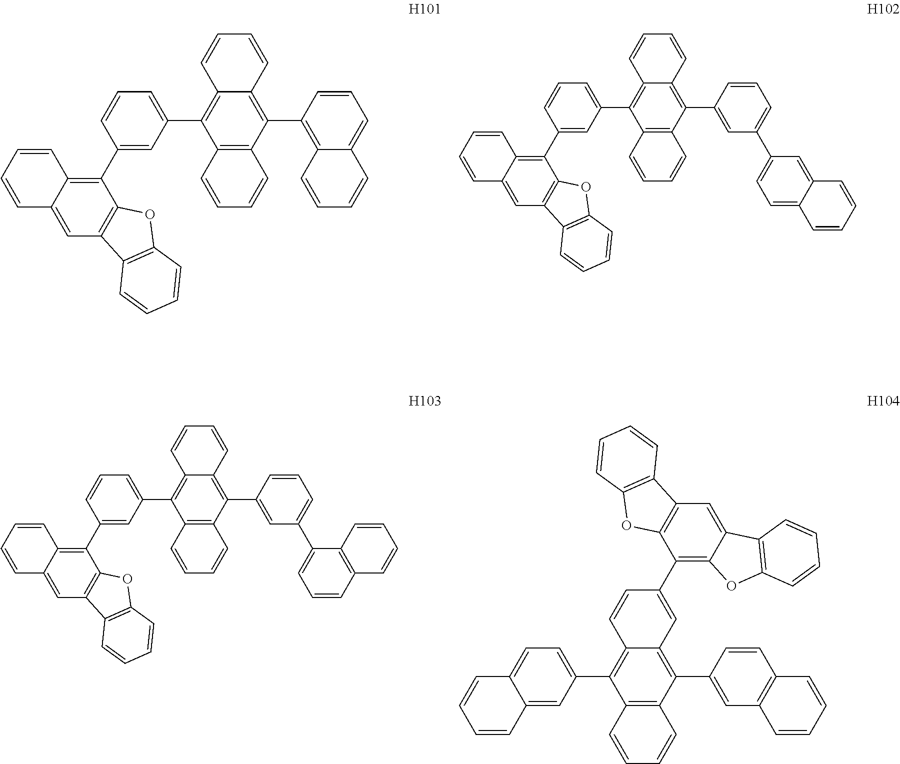 [SMILES: c1cc(-c2c3ccccc3c(-c3cccc4ccccc34)c3ccccc23)cc(-c2c3ccccc3cc3c2oc2ccccc23)c1 ; c1cc(-c2ccc3ccccc3c2)cc(-c2c3ccccc3c(-c3cccc(-c4c5ccccc5cc5c4oc4ccccc45)c3)c3ccccc23)c1 ; c1cc(-c2cccc3ccccc23)cc(-c2c3ccccc3c(-c3cccc(-c4c5ccccc5cc5c4oc4ccccc45)c3)c3ccccc23)c1 ; c1ccc2cc(-c3c4ccccc4c(-c4ccc5ccccc5c4)c4cc(-c5c6oc7ccccc7c6cc6c5oc5ccccc56)ccc34)ccc2c1]

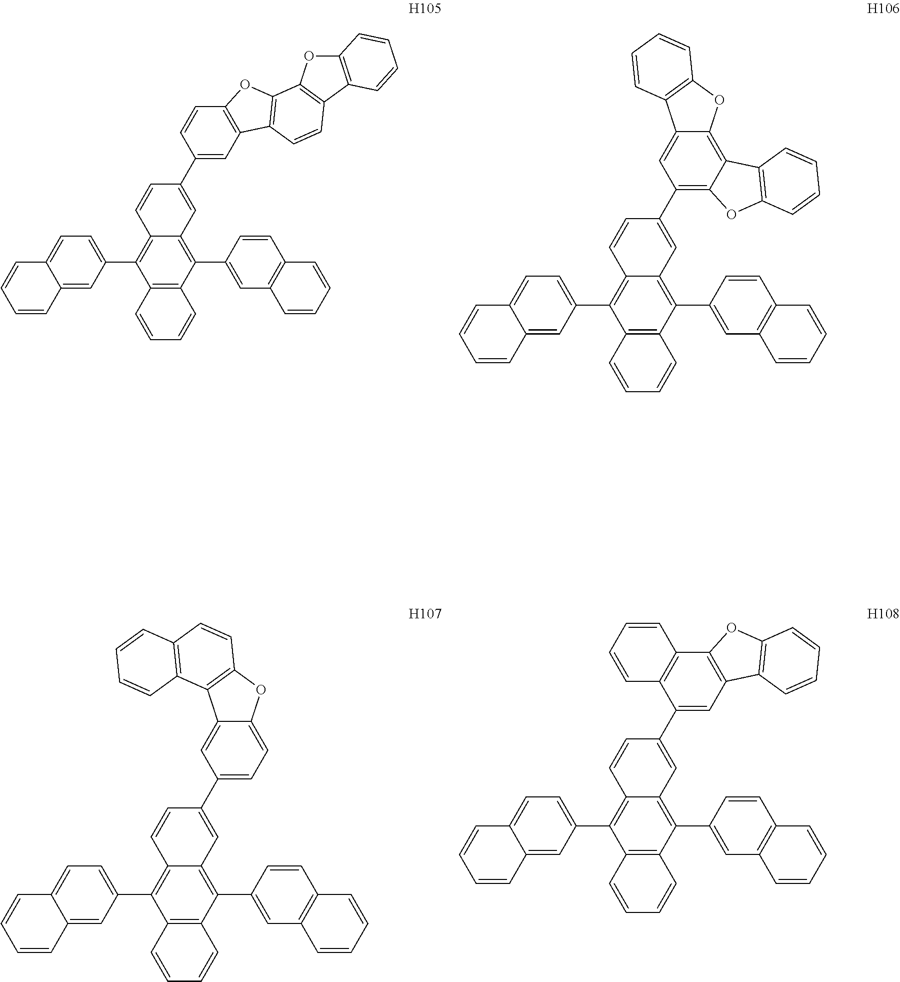 [SMILES: c1ccc2cc(-c3c4ccccc4c(-c4ccc5ccccc5c4)c4cc(-c5ccc6oc7c(ccc8c9ccccc9oc87)c6c5)ccc34)ccc2c1 ; c1ccc2cc(-c3c4ccccc4c(-c4ccc5ccccc5c4)c4cc(-c5cc6c7ccccc7oc6c6c5oc5ccccc56)ccc34)ccc2c1 ; c1ccc2cc(-c3c4ccccc4c(-c4ccc5ccccc5c4)c4cc(-c5ccc6oc7ccc8ccccc8c7c6c5)ccc34)ccc2c1 ; c1ccc2cc(-c3c4ccccc4c(-c4ccc5ccccc5c4)c4cc(-c5cc6c7ccccc7oc6c6ccccc56)ccc34)ccc2c1]

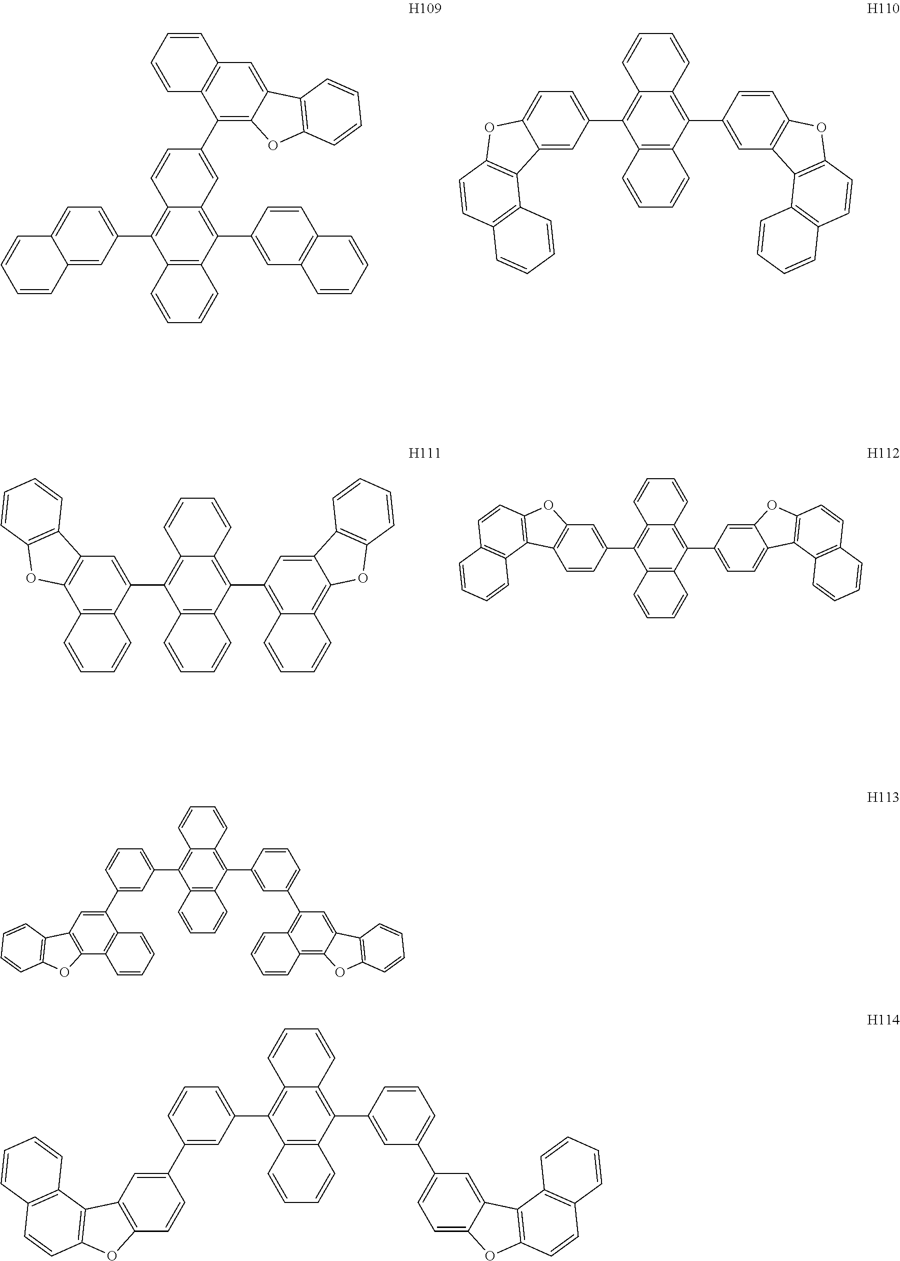

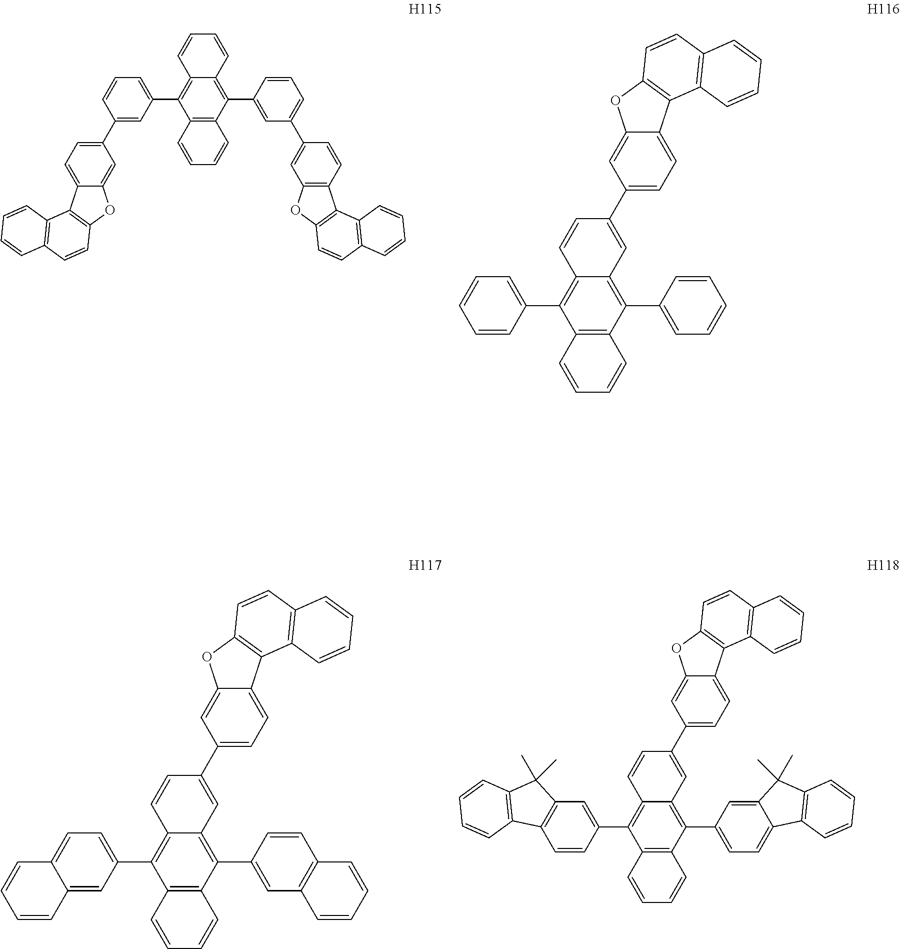 [SMILES: c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1 ; c1ccc(-c2c3ccccc3c(-c3ccccc3)c3cc(-c4ccc5c(c4)oc4ccc6ccccc6c45)ccc23)cc1 ; c1ccc2cc(-c3c4ccccc4c(-c4ccc5ccccc5c4)c4cc(-c5ccc6c(c5)oc5ccc7ccccc7c56)ccc34)ccc2c1 ; CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21]

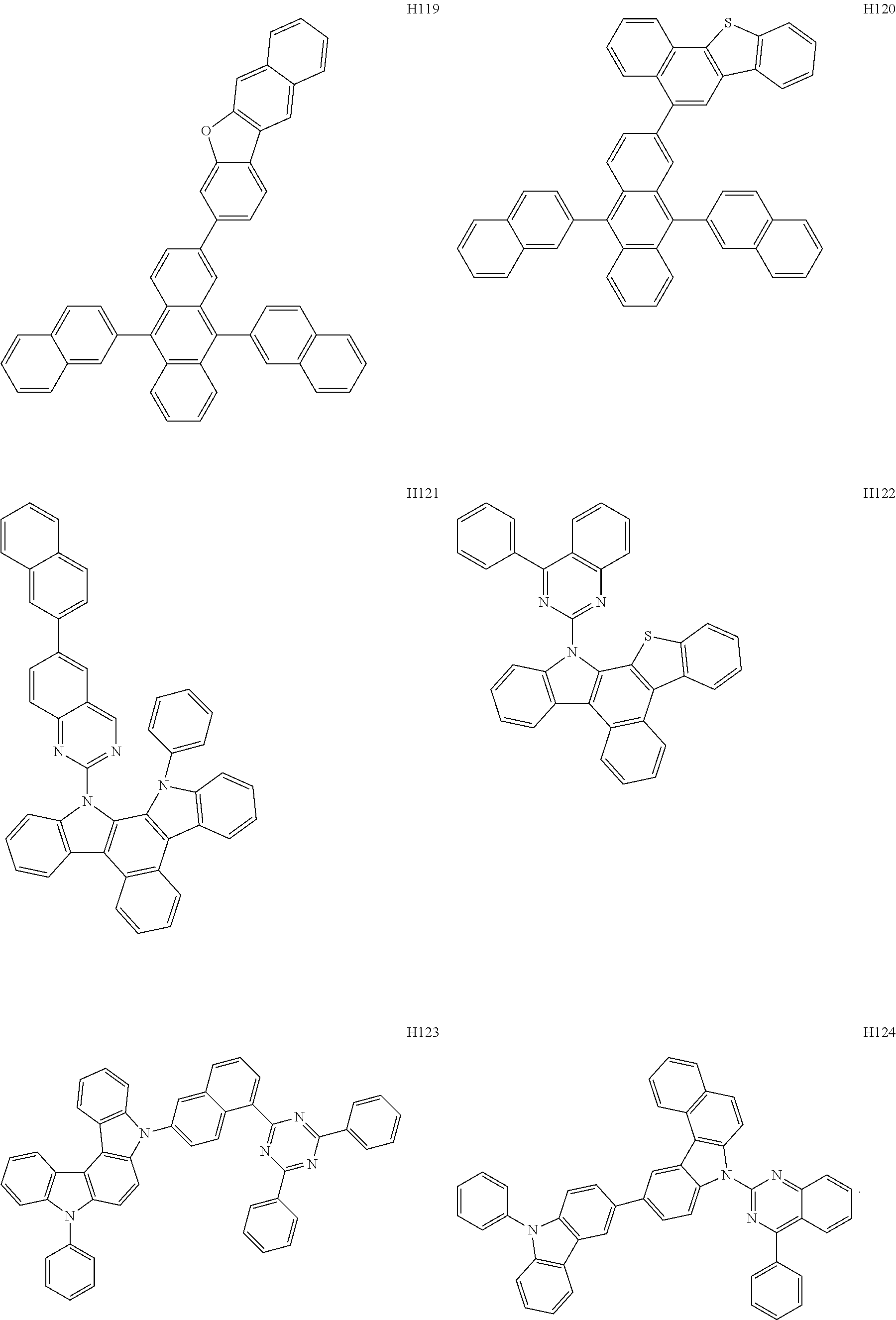

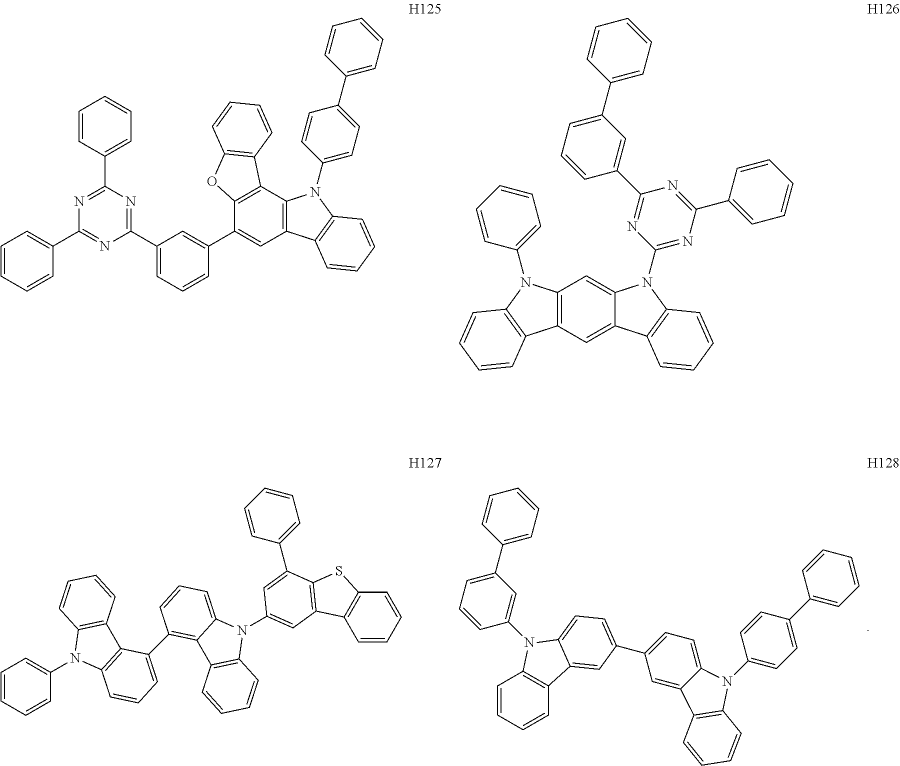 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc(-c2cc(-n3c4ccccc4c4c(-c5cccc6c5c5ccccc5n6-c5ccccc5)cccc43)cc3c2sc2ccccc23)cc1 ; c1ccc(-c2ccc(-n3c4ccccc4c4cc(-c5ccc6c(c5)c5ccccc5n6-c5cccc(-c6ccccc6)c5)ccc43)cc2)cc1]

### Phosphorescent Dopant

The phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, or any combination thereof.

The phosphorescent dopant may be electrically neutral.

For example, the phosphorescent dopant may include an organometallic compound represented by Formula 401:

M(L₄₀₁)_{xc1}(L₄₀₂)_{xc2} Formula 401

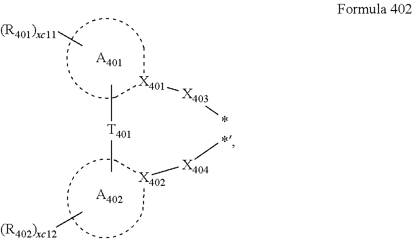

wherein, in Formulae 401 and 402,

M may be a transition metal (e.g., iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),

L₄₀₁ may be a ligand represented by Formula 402, and xc1 may be 1, 2, or 3, wherein, when xc1 is 2 or more, two or more of L₄₀₁ (s) may be identical to or different from each other,

L₄₀₂ may be an organic ligand, and xc2 may be 0, 1, 2, 3, or 4, wherein, when xc2 is 2 or more, two or more of L₄₀₂(s) may be identical to or different from each other,

X₄₀₁ and X₄₀₂ may each independently be nitrogen or carbon,

ring A₄₀₁ and ring A₄₀₂ may each independently be a C₃-C₆₀ carbocyclic group or a C₁-C₆₀ heterocyclic group,
T₄₀₁ may be a single bond, *—O—*′, *—S—*′, *—C(═O)—*′, *—N(Q₄₁₁)-*′, *—C(Q₄₁₁)(Q₄₁₂)-*′, *—C(Q₄₁₁)=C(Q₄₁₂)-*′, *—C(Q₄₁₁)=*′, or *═C=*′,
X₄₀₃ and X₄₀₄ may each independently be a chemical bond (e.g., a covalent bond or a coordinate bond (which may also be referred to as a coordinate covalent bond or a dative bond)), O, S, N(Q₄₁₃), B(Q₄₁₃), P(Q₄₁₃), C(Q₄₁₃)(Q₄₁₄), or Si(Q₄₁₃)(Q₄₁₄),

Q₄₁₁ to Q₄₁₄ may each be as defined herein in connection with Q₁,

R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),

Q₄₀₁ to Q₄₀₃ may each be as defined herein in connection with Q₁,

xc11 and xc12 may each independently be an integer from 0 to 10, and

* and *′ in Formula 402 may each indicate a binding site to M in Formula 401.

For example, in Formula 402, i) X₄₀₁ may be nitrogen, and X₄₀₂ may be carbon, or ii) X₄₀₁ and X₄₀₂ may each be nitrogen.

In one or more embodiments, when xc1 in Formula 401 is 2 or more, two ring A₄₀₁(s) in two or more of L₄₀₁(s) may optionally be linked to each other via T₄₀₂, which is a linking group, and two ring A₄₀₂(s) may optionally be linked to each other via T₄₀₃, which is a linking group (see Compounds PD1 to PD4 and PD7). T₄₀₂ and T₄₀₃ may each be as defined herein in connection with T₄₀₁.

L₄₀₂ in Formula 401 may be an organic ligand. For example, L₄₀₂ may include a halogen group, a diketone group (e.g., an acetylacetonate group), a carboxylic acid group (e.g., a picolinate group), —C(═O) group, an isonitrile group, a —CN group, a phosphorus-containing group (e.g., a phosphine group, a phosphite group, etc.), or any combination thereof.

The phosphorescent dopant may include, for example, one selected from Compounds PD1 to PD39 or any combination thereof:

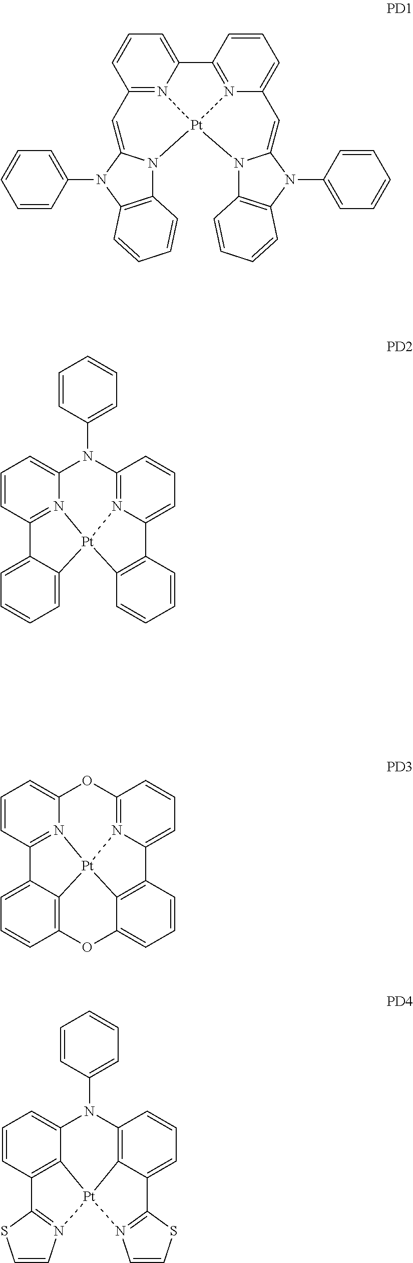

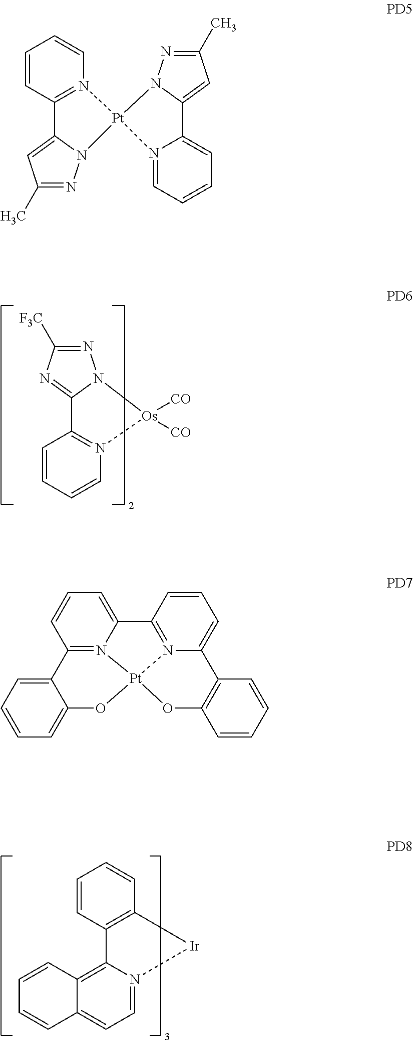

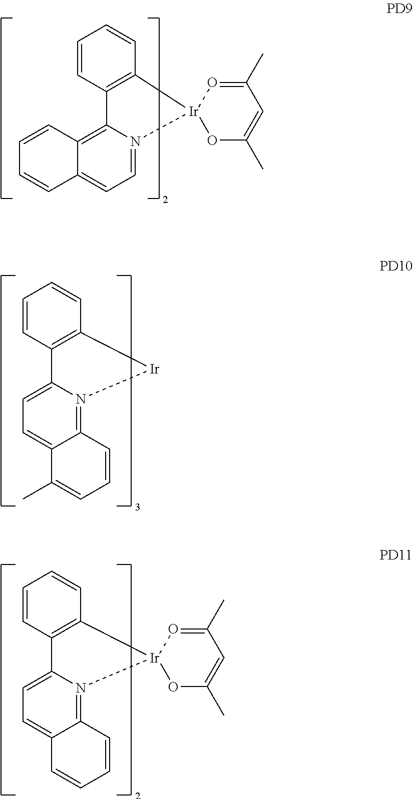

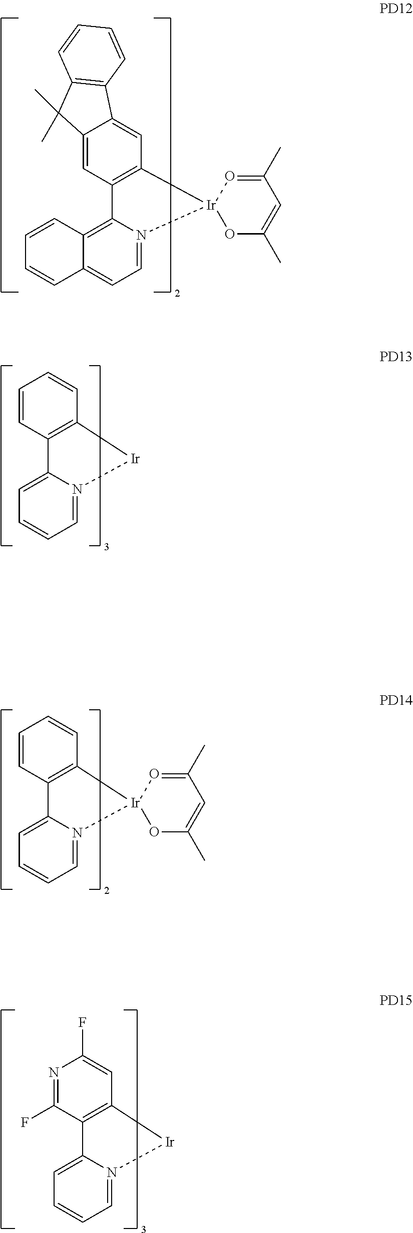

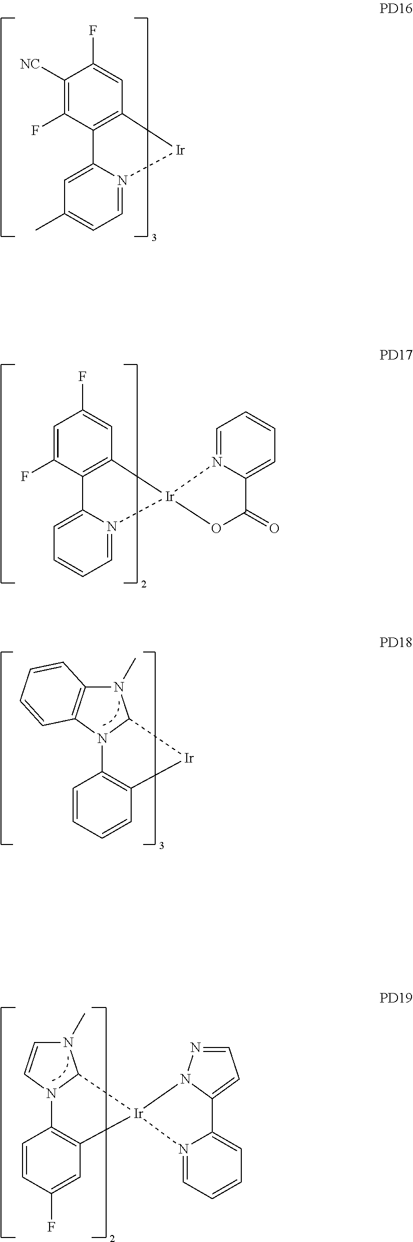

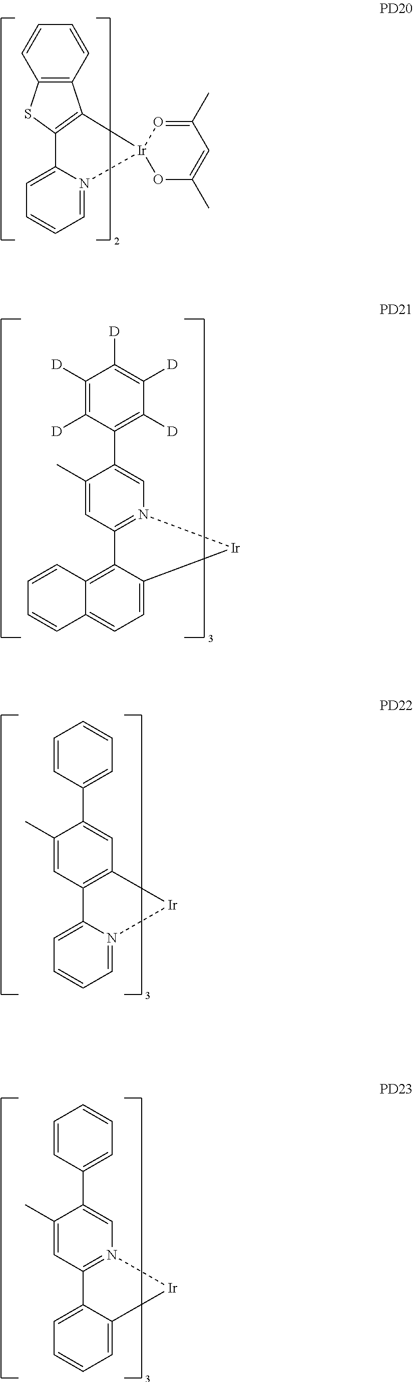

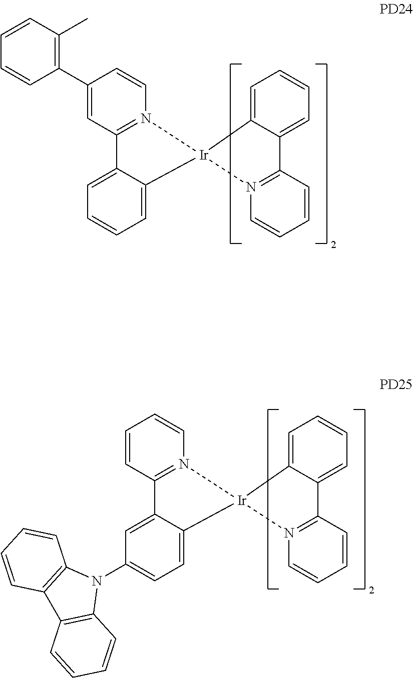

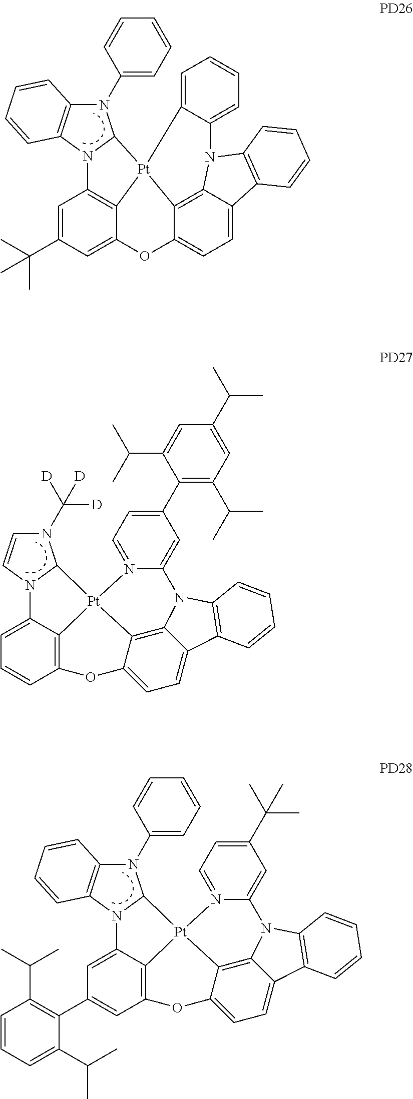

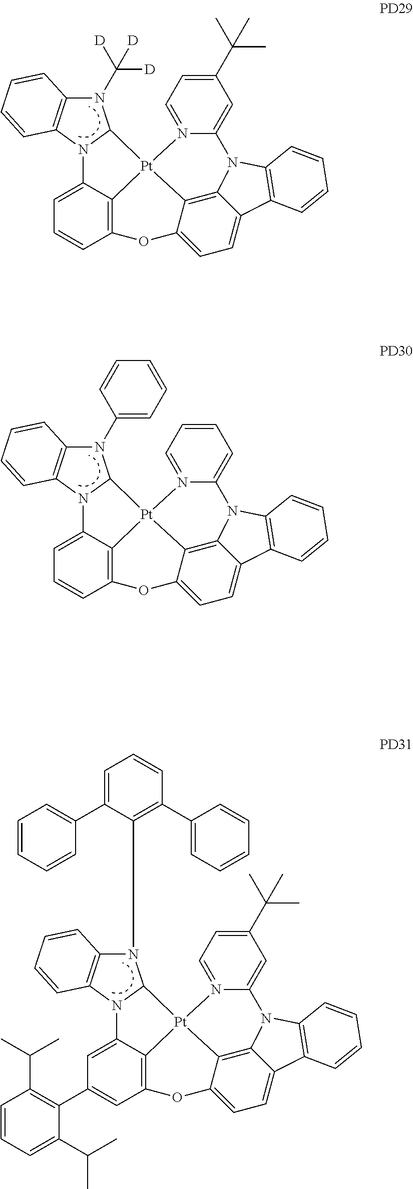

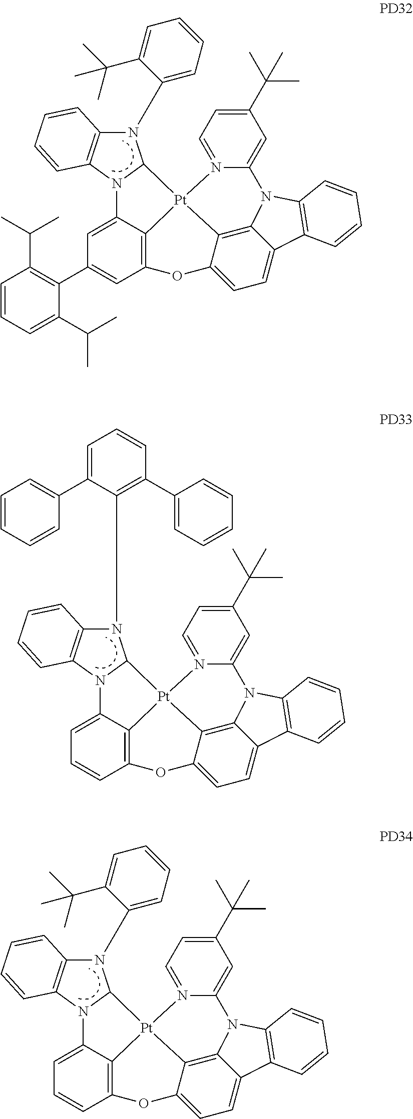

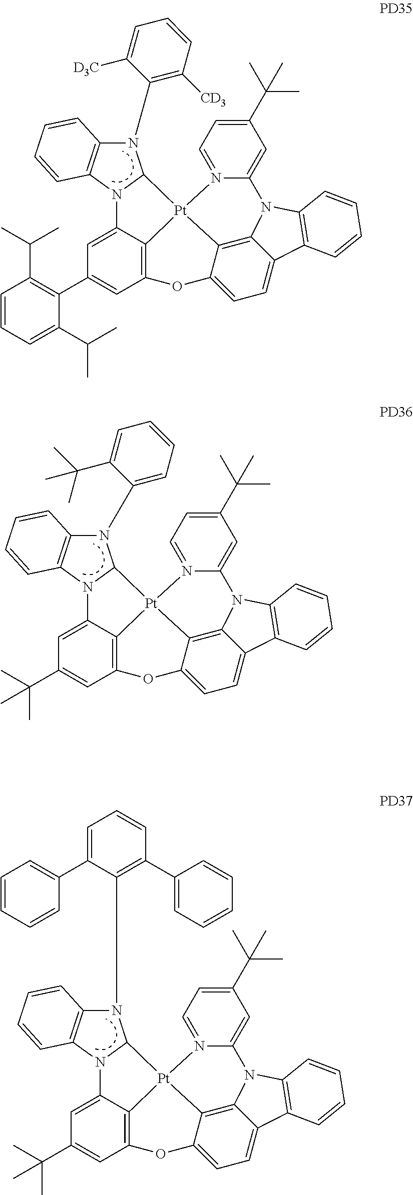

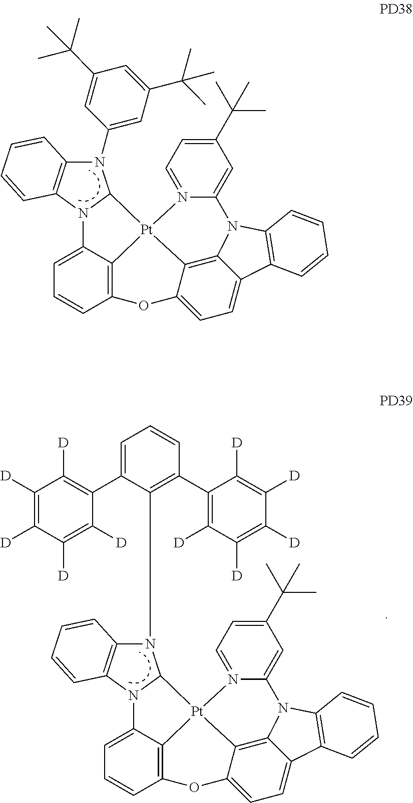

### Fluorescent Dopant

The fluorescent dopant may include an amine group-containing compound, a styryl group-containing compound, or any combination thereof.

For example, the fluorescent dopant may include a compound represented by Formula 501:

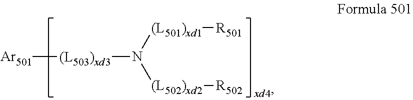

wherein, in Formula 501,

Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xd1 to xd3 may each independently be 0, 1, 2, or 3, and

xd4 may be 1, 2, 3, 4, 5, or 6.

For example, Ar₅₀₁ in Formula 501 may be a condensed cyclic group (e.g., an anthracene group, a chrysene group, a pyrene group, etc.) in which three or more monocyclic groups are condensed together with each other.

In one or more embodiments, xd4 in Formula 501 may be 2.

For example, the fluorescent dopant may include one selected from Compounds FD1 to FD37, DPVBi and DPAVBi, or any combination thereof:

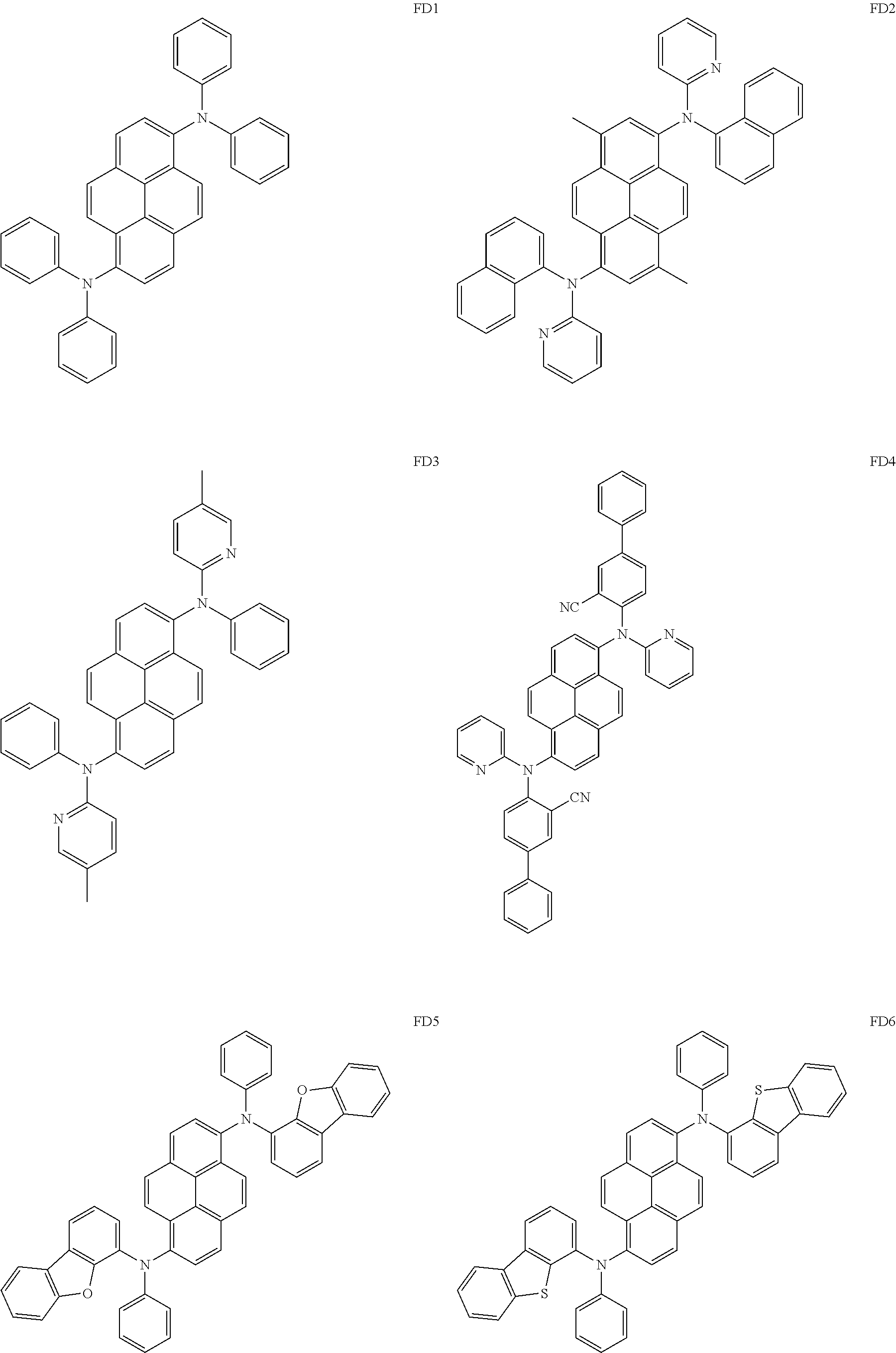

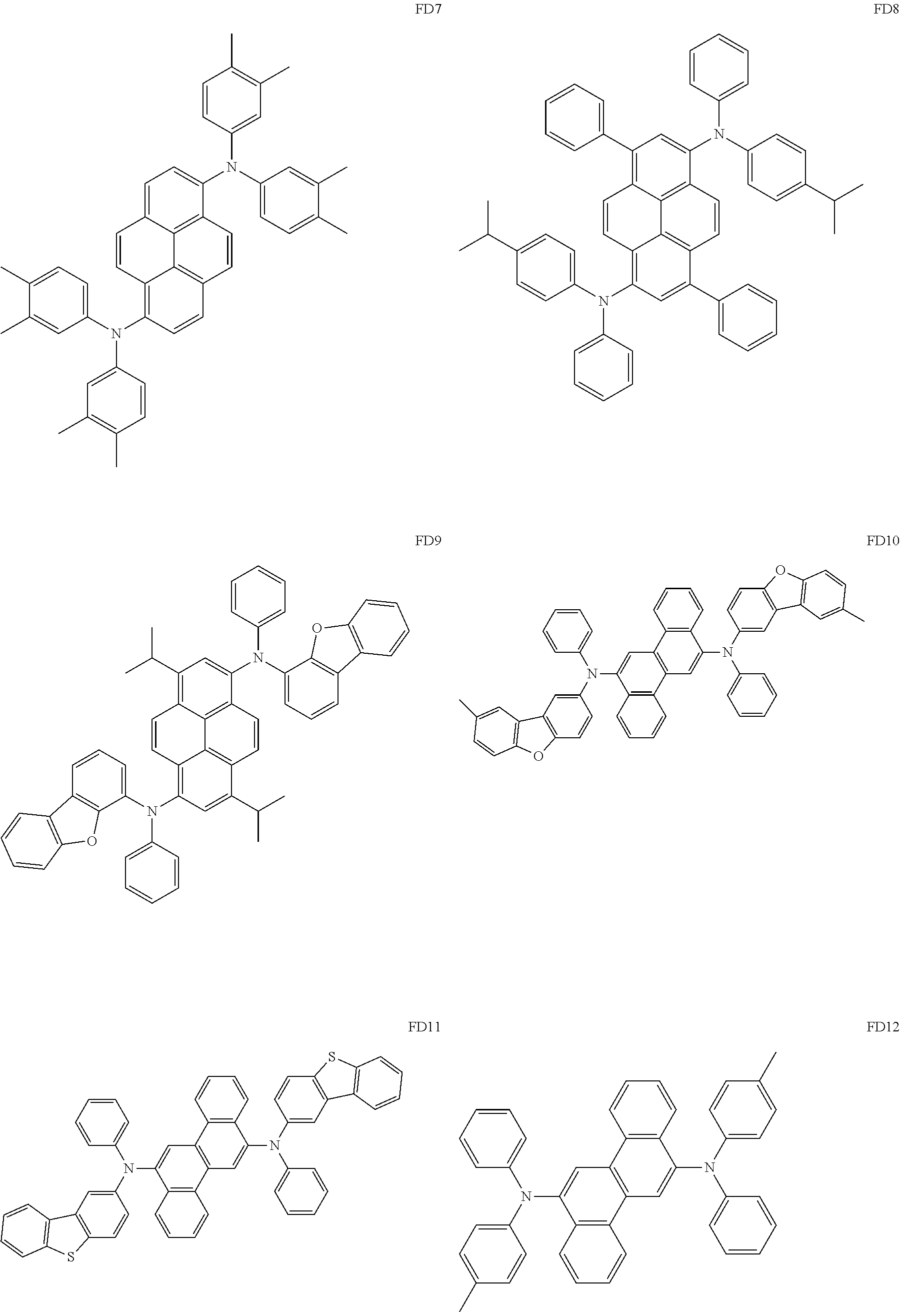

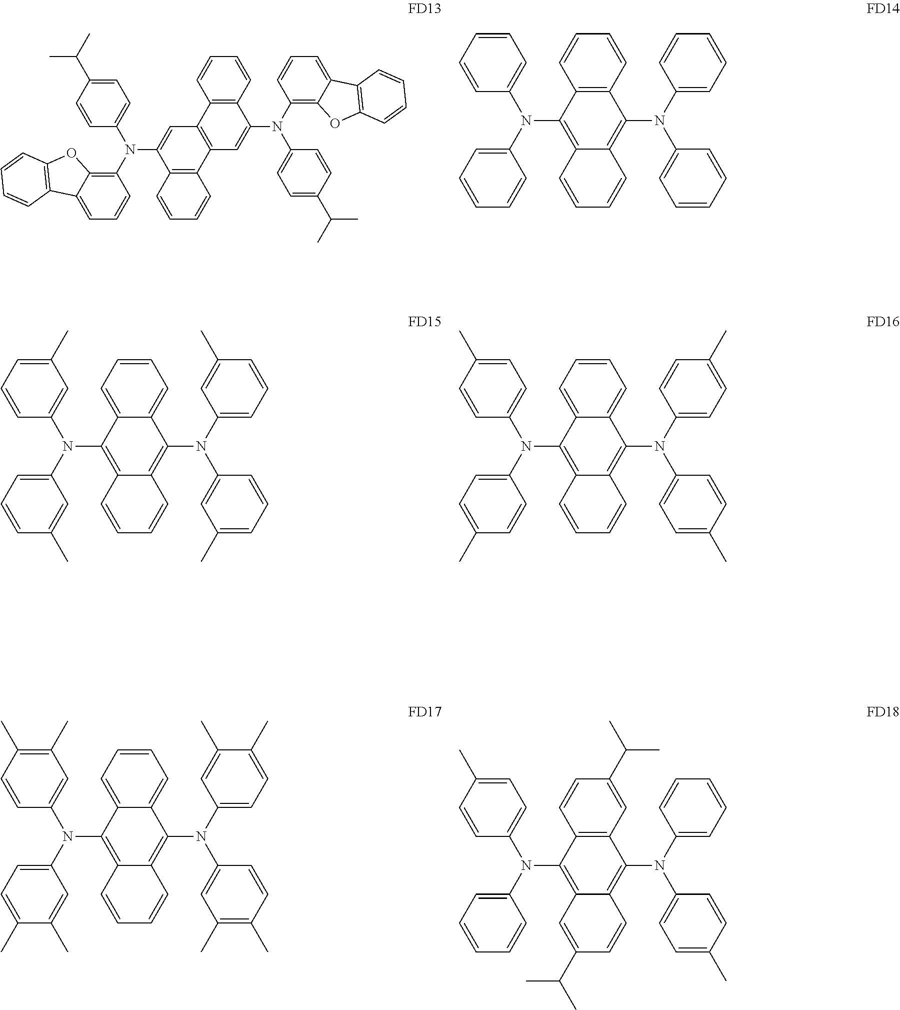 [SMILES: CC(C)c1ccc(N(c2cc3c4ccccc4c(N(c4ccc(C(C)C)cc4)c4cccc5c4oc4ccccc45)cc3c3ccccc23)c2cccc3c2oc2ccccc23)cc1 ; c1ccc(N(c2ccccc2)c2c3ccccc3c(N(c3ccccc3)c3ccccc3)c3ccccc23)cc1 ; Cc1cccc(N(c2cccc(C)c2)c2c3ccccc3c(N(c3cccc(C)c3)c3cccc(C)c3)c3ccccc23)c1 ; Cc1ccc(N(c2ccc(C)cc2)c2c3ccccc3c(N(c3ccc(C)cc3)c3ccc(C)cc3)c3ccccc23)cc1 ; Cc1ccc(N(c2ccc(C)c(C)c2)c2c3ccccc3c(N(c3ccc(C)c(C)c3)c3ccc(C)c(C)c3)c3ccccc23)cc1C ; Cc1ccc(N(c2ccccc2)c2c3ccc(C(C)C)cc3c(N(c3ccccc3)c3ccc(C)cc3)c3ccc(C(C)C)cc23)cc1]

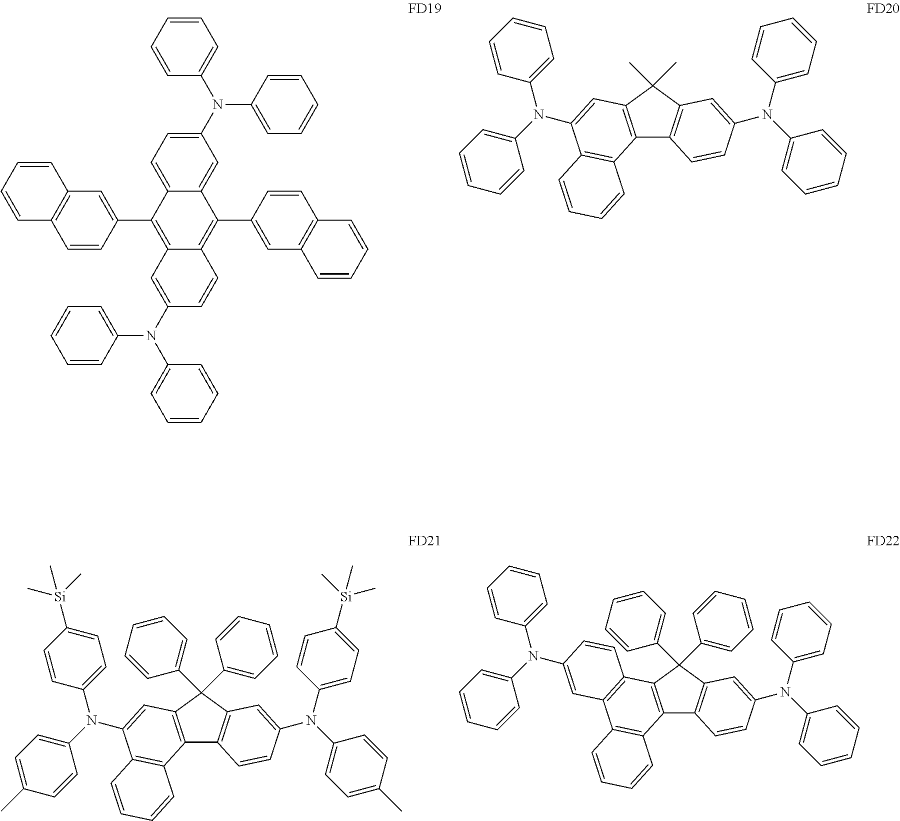

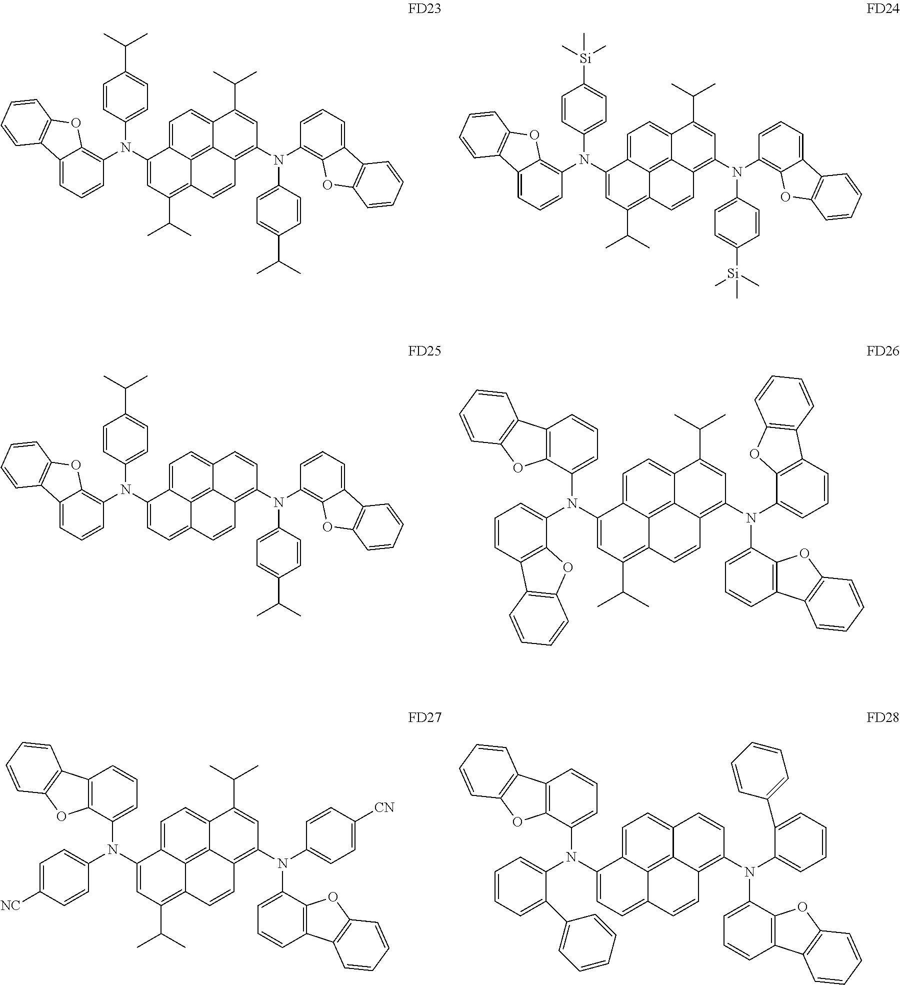

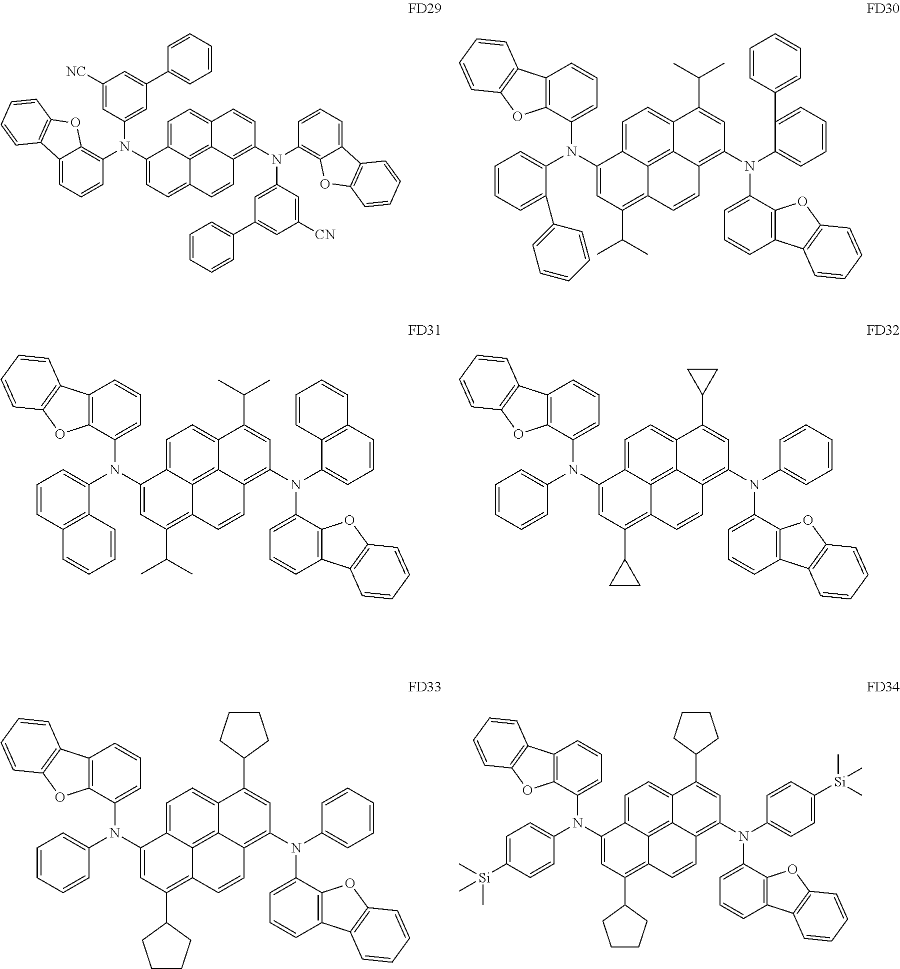

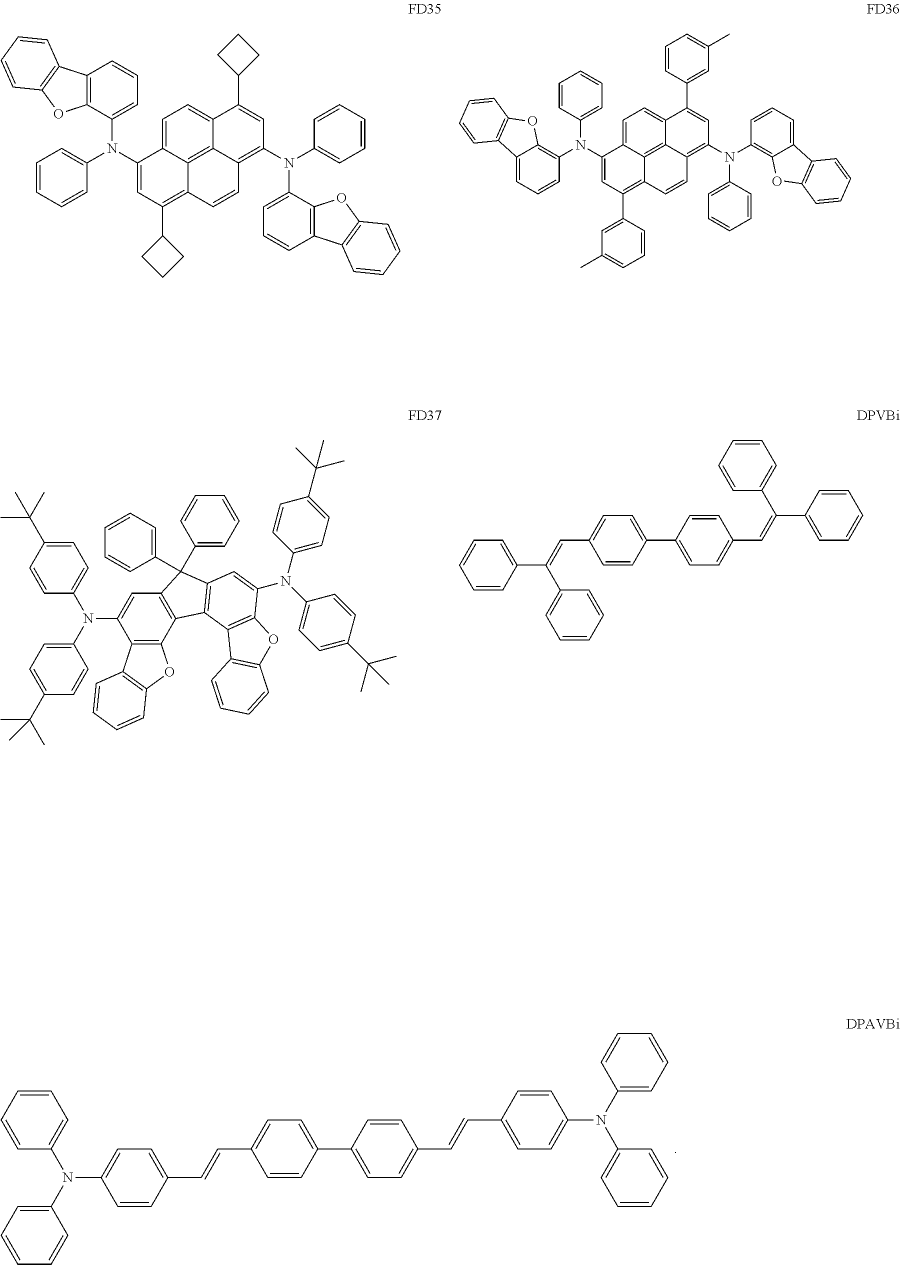

### Delayed Fluorescence Material

The emission layer 130 may include a delayed fluorescence material.

In the present specification, the delayed fluorescence material may be selected from compounds capable of emitting delayed fluorescent light based on a delayed fluorescence emission mechanism.

The delayed fluorescence material included in the emission layer 130 may act as a host or a dopant depending on the type (or kind) of other materials included in the emission layer 130.

In one or more embodiments, a difference between a triplet energy level (eV) of the delayed fluorescence material and a singlet energy level (eV) of the delayed fluorescence material may be 0 eV or more and 0.5 eV or less. When the difference between the triplet energy level (eV) of the delayed fluorescence material and the singlet energy level (eV) of the delayed fluorescence material satisfies the above-described range, up-conversion from the triplet state to the singlet state of the delayed fluorescence materials may effectively occur, and thus, the luminescence efficiency of the light-emitting device 10 may be improved.

For example, the delayed fluorescence material may include i) a material including at least one electron donor (e.g., a π electron-rich C₃-C₆₀ cyclic group, such as a carbazole group, etc.) and at least one electron acceptor (e.g., a sulfoxide group, a cyano group, a π electron-deficient nitrogen-containing C₁-C₆₀ cyclic group, etc.), and ii) a material including a C₈-C₆₀ polycyclic group in which two or more cyclic groups are condensed together while sharing boron (B).

Examples of the delayed fluorescence material may include at least one selected from Compounds DF1 to DF14:

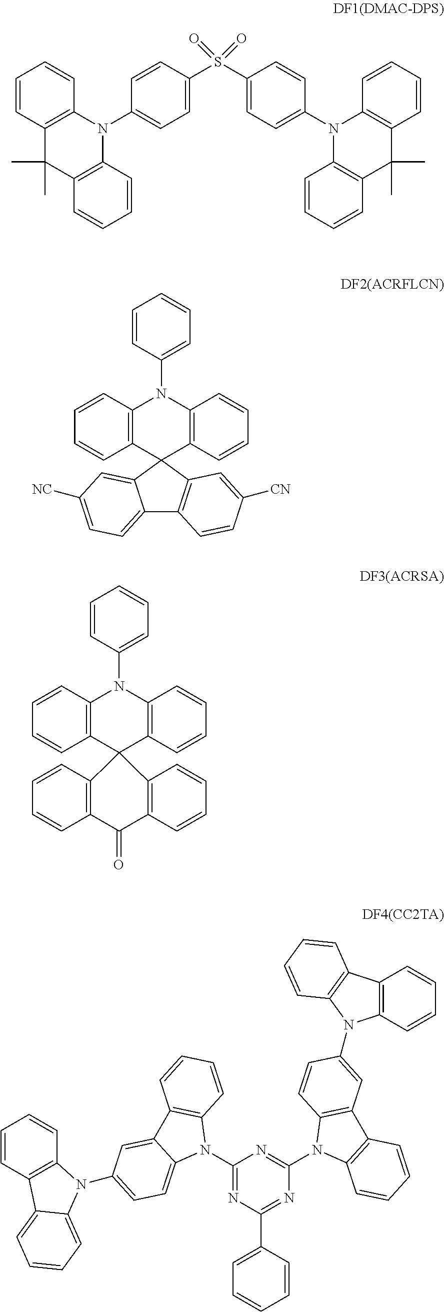

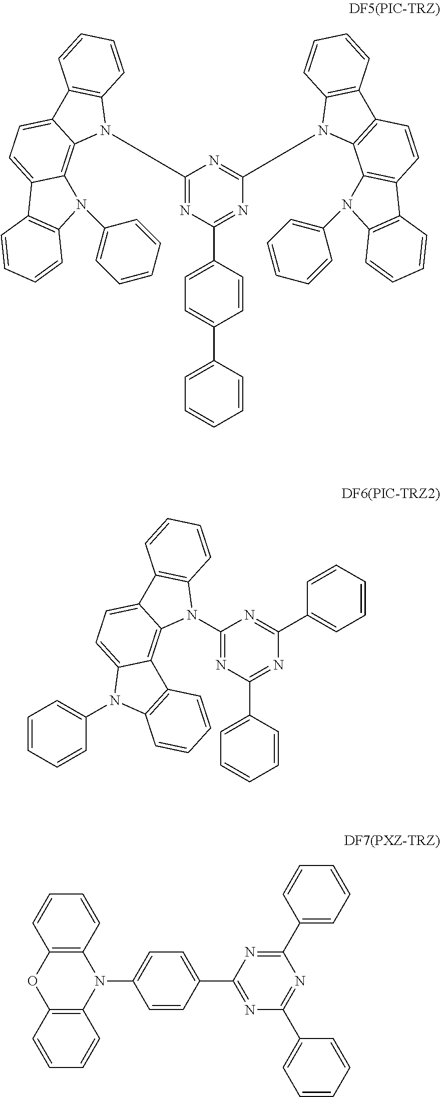 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-n3c4ccccc4c4ccc5c(c6ccccc6n5-c5ccccc5)c43)n2)cc1 ; c1ccc(-c2nc(-c3ccccc3)nc(-c3ccc(N4c5ccccc5Oc5ccccc54)cc3)n2)cc1]

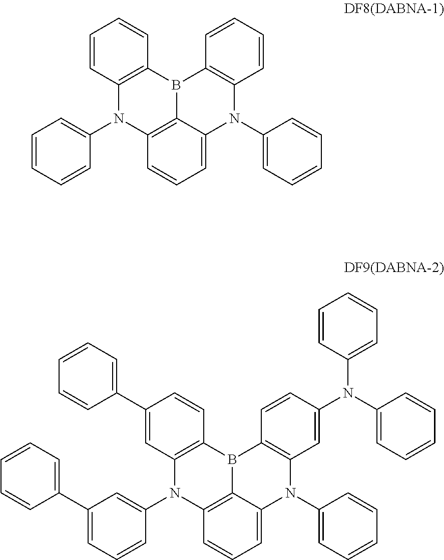 [SMILES: c1ccc(N2c3ccccc3B3c4ccccc4N(c4ccccc4)c4cccc2c43)cc1 ; c1ccc(-c2cccc(N3c4cc(-c5ccccc5)ccc4B4c5ccc(N(c6ccccc6)c6ccccc6)cc5N(c5ccccc5)c5cccc3c54)c2)cc1]

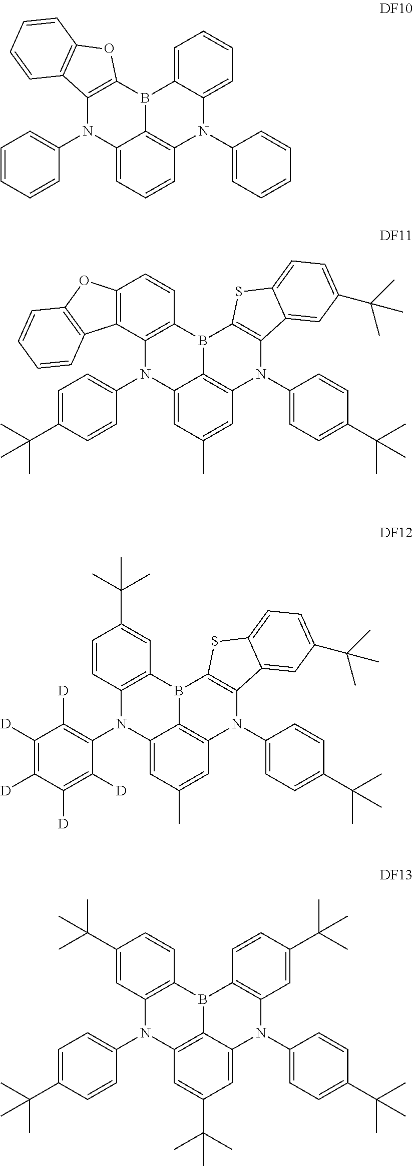

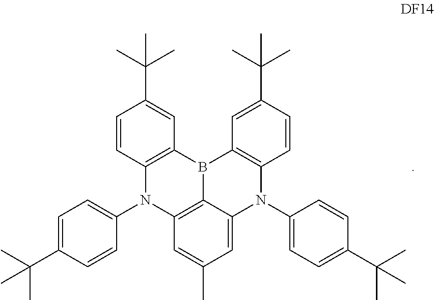 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1]

### Quantum Dot

The emission layer 130 may include a quantum dot.

The term “quantum dot” as used herein refers to a crystal of a semiconductor compound, and may include any suitable material capable of emitting light of various suitable emission wavelengths according to the size of the crystal.

A diameter of the quantum dot may be, for example, in a range of about 1 nm to about 10 nm.

The quantum dot may be synthesized by a wet chemical process, a metal organic chemical vapor deposition process, a molecular beam epitaxy process, and/or any suitable process similar thereto.

The wet chemical process is a method including mixing a precursor material together with an organic solvent and then growing a quantum dot particle crystal. When the crystal grows, the organic solvent naturally acts as a dispersant coordinated on the surface of the quantum dot crystal and controls the growth of the crystal so that the growth of quantum dot particles can be controlled through a process which costs lower, and is easier than vapor deposition methods, such as metal organic chemical vapor deposition (MOCVD) or molecular beam epitaxy (MBE).

The quantum dot may include a Group II-VI semiconductor compound, a Group III-V semiconductor compound, a Group III-VI semiconductor compound, a Group I-III-VI semiconductor compound, a Group IV-VI semiconductor compound, a Group IV element or compound, or any combination thereof.

Examples of the Group II-VI semiconductor compound may include: a binary compound, such as CdS, CdSe, CdTe, ZnS, ZnSe, ZnTe, ZnO, HgS, HgSe, HgTe, MgSe, and/or MgS; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, and/or MgZnS; a quaternary compound, such as CdZnSeS, CdZnSeTe, CdZnSTe, CdHgSeS, CdHgSeTe, CdHgSTe, HgZnSeS, HgZnSeTe, and/or HgZnSTe; or any combination thereof.

Examples of the Group III-V semiconductor compound may include: a binary compound, such as GaN, GaP, GaAs, GaSb, AlN, AlP, AlAs, AlSb, InN, InP, InAs, and/or InSb; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, and/or InPSb; a quaternary compound, such as GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, and/or InAlPSb; or any combination thereof. In one or more embodiments, the Group III-V semiconductor compound may further include a Group II element. Examples of the Group III-V semiconductor compound further including a Group II element may include InZnP, InGaZnP, InAlZnP, and the like.

Examples of the Group III-VI semiconductor compound may include: a binary compound, such as GaS, GaSe, Ga₂Se₃, GaTe, InS, InSe, In₂S₃, In₂Se₃, and/or InTe; a ternary compound, such as InGaS₃ and/or InGaSe₃; or any combination thereof.

Examples of the Group I-III-VI semiconductor compound may include: a ternary compound, such as AgInS, AgInS₂, CuInS, CuInS₂, CuGaO₂, AgGaO₂, and/or AgAlO₂; or any combination thereof.

Examples of the Group IV-VI semiconductor compound may include: a binary compound, such as SnS, SnSe, SnTe, PbS, PbSe, and/or PbTe; a ternary compound, such as SnSeS, SnSeTe, SnSTe, PbSeS, PbSeTe, PbSTe, SnPbS, SnPbSe, and/or SnPbTe; a quaternary compound, such as SnPbSSe, SnPbSeTe, and/or SnPbSTe; or any combination thereof.

The Group IV element or compound may include: a single element, such as Si or Ge; a binary compound, such as SiC and/or SiGe; or any combination thereof.

Each element included in a multi-element compound, such as the binary compound, the ternary compound, and the quaternary compound, may be present at a uniform concentration or non-uniform concentration in a particle.

In one or more embodiments, the quantum dot may have a single structure in which the concentration of each element in the quantum dot is uniform (e.g., substantially uniform), or a core-shell dual structure. For example, a material included in the core and a material included in the shell may be different from each other.

The shell of the quantum dot may act as a protective layer that prevents or reduces chemical degeneration of the core to maintain semiconductor characteristics, and/or as a charging layer that imparts electrophoretic characteristics to the quantum dot. The shell may be a single layer or a multi-layer. The interface between the core and the shell may have a concentration gradient in which the concentration of an element existing in the shell decreases along a direction toward the center of the core.

Examples of the shell of the quantum dot may include an oxide of metal, metalloid, and/or non-metal, a semiconductor compound, or any combination thereof. Examples of the oxide of metal, metalloid, and/or non-metal may include: a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, and/or NiO; a ternary compound, such as MgAl₂O₄, CoFe₂O₄, NiFe₂O₄, and/or CoMn₂O₄; or any combination thereof. Examples of the semiconductor compound may include, as described herein, a Group II-VI semiconductor compound, a Group III-V semiconductor compound, a Group III-VI semiconductor compound, a Group I-III-VI semiconductor compound, a Group IV-VI semiconductor compound, or any combination thereof. For example, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, or any combination thereof.

A full width at half maximum (FWHM) of an emission wavelength spectrum of the quantum dot may be about 45 nm or less, for example, about 40 nm or less, for example, about 30 nm or less, and within these ranges, color purity and/or color reproducibility may be improved. In addition, because the light emitted through the quantum dot is emitted in all directions, the wide viewing angle may be improved.

In addition, the quantum dot may be in the form of a spherical particle, a pyramidal particle, a multi-arm particle, a cubic nanoparticle, a nanotube particle, a nanowire particle, a nanofiber particle, and/or a nanoplate particle.

Because the energy band gap may be adjusted by controlling the size of the quantum dot, light having various suitable wavelength bands may be obtained from an emission layer including the quantum dot. Accordingly, by using quantum dots of different sizes, a light-emitting device that emits light of various suitable wavelengths may be implemented. In more detail, the size of the quantum dot may be selected to emit red, green and/or blue light. In addition, the size of the quantum dot may be configured to emit white light by combining light of various suitable colors.

### Photoactive Layer 135

The opto-electronic device 30 may include the photoactive layer 135 arranged on the hole transport region 120. The photoactive layer 135 may include a first layer 131 adjacent to the hole transport region 120 and a second layer 132 adjacent to the electron transport region 140.

The emission layer 130 may emit light to the outside of an electronic apparatus. The light may be reflected by an external object to be incident on the electronic apparatus.

The photoactive layer 135 may generate an electrical signal by absorbing the light incident on the electronic apparatus. Accordingly, the opto-electronic device 30 including the photoactive layer 135 may serve as a photosensor.

### Electron Transport Region 140

The electron transport region 140 may be arranged on the emission layer 130 of the light-emitting device 10 and the photoactive layer 135 of the opto-electronic device 30. The electron transport region 140 may have i) a single-layered structure consisting of a single layer consisting of a single material, ii) a single-layered structure consisting of a single layer including a plurality of different materials, or iii) a multi-layered structure including a plurality of layers including different materials.

The electron transport region 140 may include a buffer layer, a hole blocking layer, an electron control layer, an electron transport layer, an electron injection layer, or any combination thereof.

For example, the electron transport region 140 may have an electron transport layer/electron injection layer structure, a hole blocking layer/electron transport layer/electron injection layer structure, an electron control layer/electron transport layer/electron injection layer structure, or a buffer layer/electron transport layer/electron injection layer structure, the layers of each structure being sequentially stacked from the emission layer 130.

The electron transport region 140 (e.g., the buffer layer, the hole blocking layer, the electron control layer, or the electron transport layer in the electron transport region 140) may include a metal-free compound including at least one π electron-deficient nitrogen-containing C₁-C₆₀ cyclic group.

For example, the electron transport region 140 may include a compound represented by Formula 601:

[Ar₆₀₁]ₓₑ₁₁-[(L₆₀₁)ₓₑ₁-R₆₀₁]ₓₑ₂₁, Formula 601

wherein, in Formula 601,

Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xe11 may be 1, 2, or 3,

xe1 may be 0, 1, 2, 3, 4, or 5,

R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),

Q₆₀₁ to Q₆₀₃ may each be as defined herein in connection with Q₁,

xe21 may be 1, 2, 3, 4, or 5, and

at least one selected from Ar₆₀₁, L₆₀₁, and R₆₀₁ may each independently be a π electron-deficient nitrogen-containing C₁-C₆₀ cyclic group unsubstituted or substituted with at least one R₁₀ₐ.

For example, when xe11 in Formula 601 is 2 or more, two or more of Ar₆₀₁(s) may be linked to each other via a single bond.

In one or more embodiments, Ar₆₀₁ in Formula 601 may be a anthracene group unsubstituted or substituted with at least one R₁₀ₐ.

In one or more embodiments, the electron transport region 140 may include a compound represented by Formula 601-1:

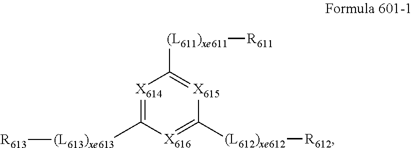

wherein, in Formula 601-1,

X₆₁₄ may be N or C(R₆₁₄), X₆₁₅ may be N or C(R₆₁₅), X₆₁₆ may be N or C(R₆₁₆), and at least one selected from X₆₁₄ to X₆₁₆ may be N,

L₆₁₁ to L₆₁₃ may each be as defined herein in connection with L₆₀₁,

xe611 to xe613 may each be as defined herein in connection with xe1,

R₆₁₁ to R₆₁₃ may each be as defined herein in connection with R₆₀₁ and

R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, xe1 and xe611 to xe613 in Formulae 601 and 601-1 may each independently be 0, 1, or 2.

The electron transport region 140 may include one selected from Compounds ET1 to ET45, 2,9-dimethyl-4,7-diphenyl-1,10-phenanthroline (BCP), 4,7-diphenyl-1,10-phenanthroline (Bphen), Alq₃, BAlq, TAZ and NTAZ, or any combination thereof:

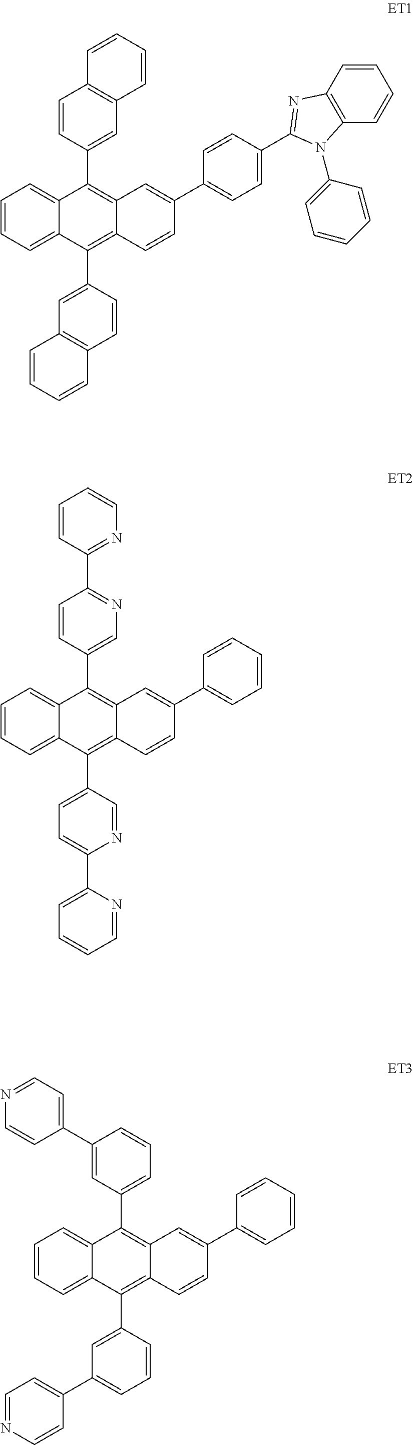 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

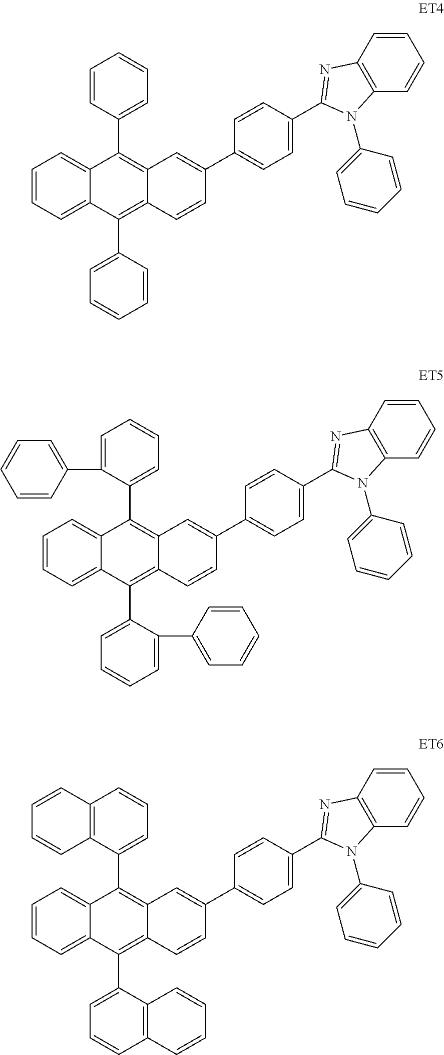 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

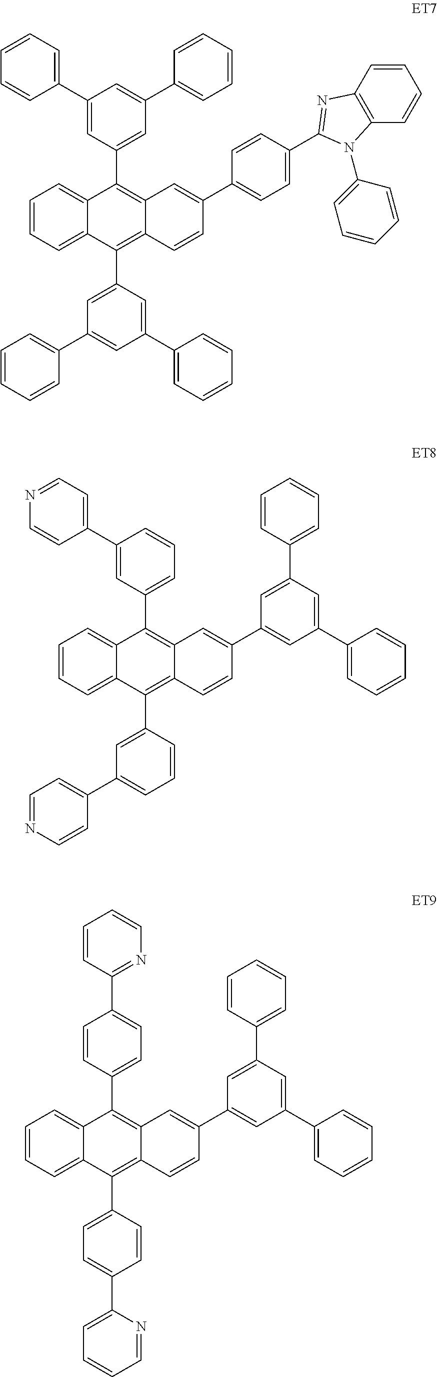 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccncc6)c5)c5ccccc5c(-c5cccc(-c6ccncc6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

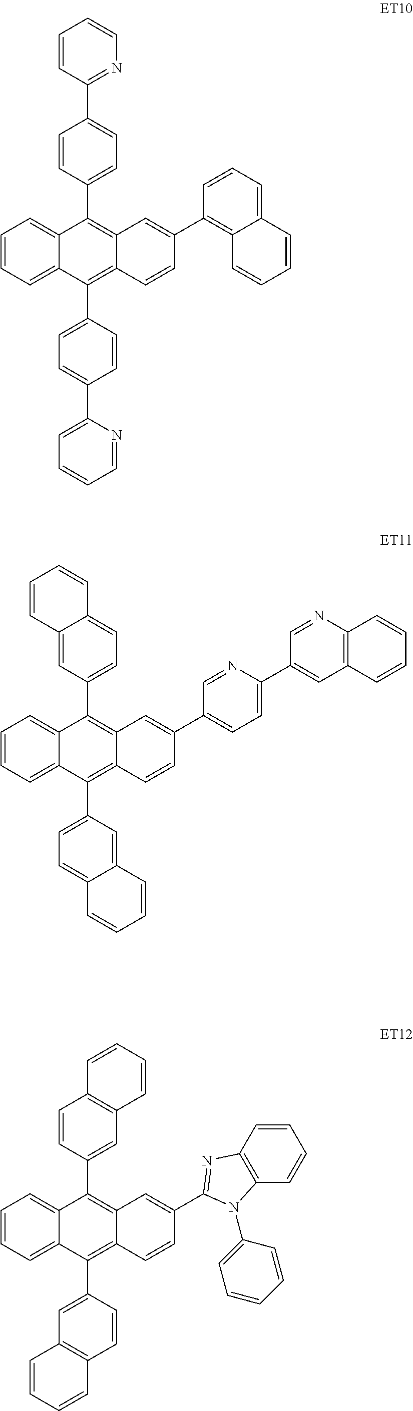 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

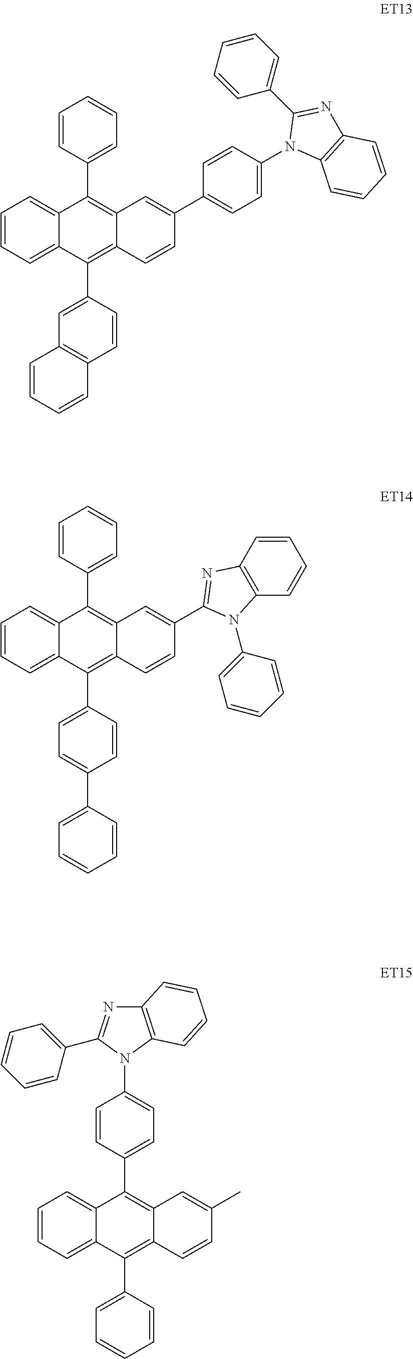 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

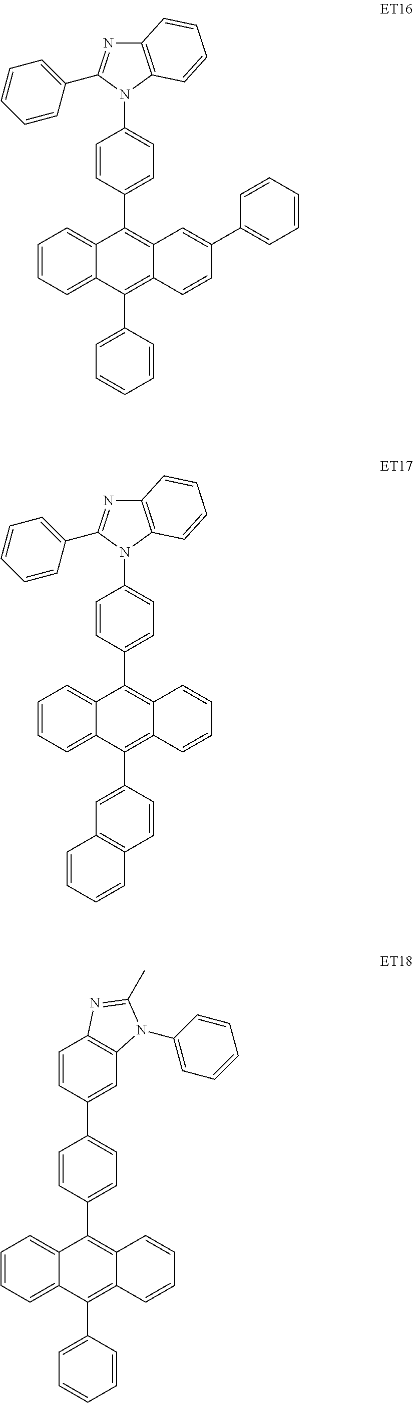 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

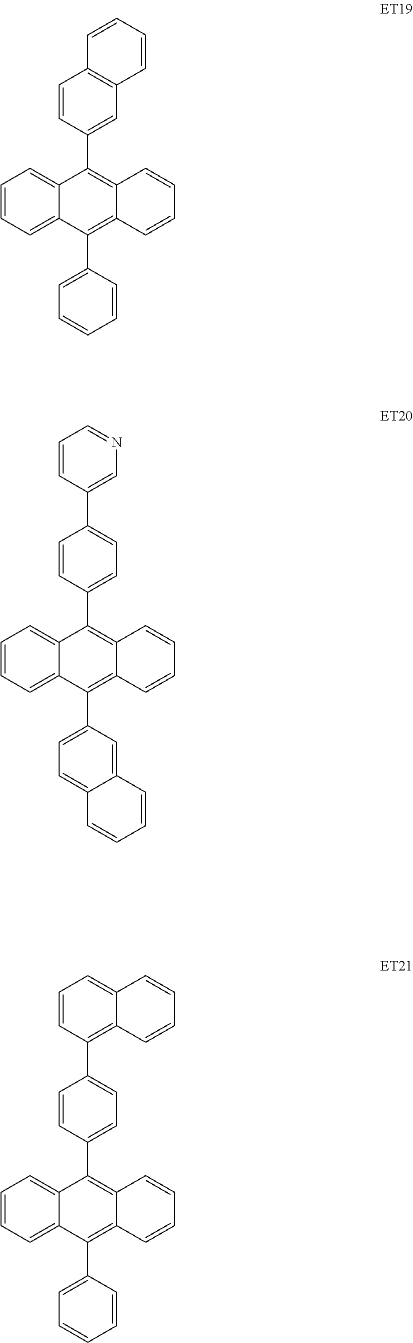

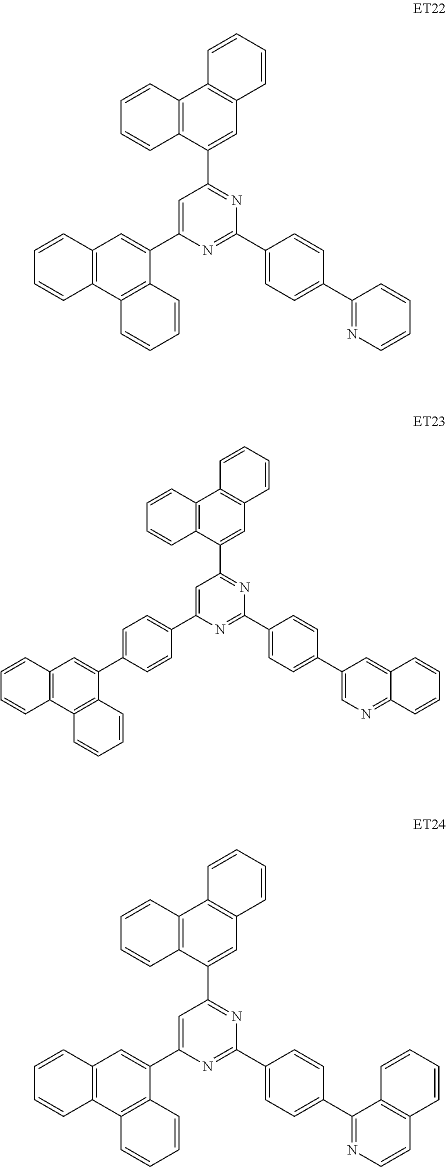 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

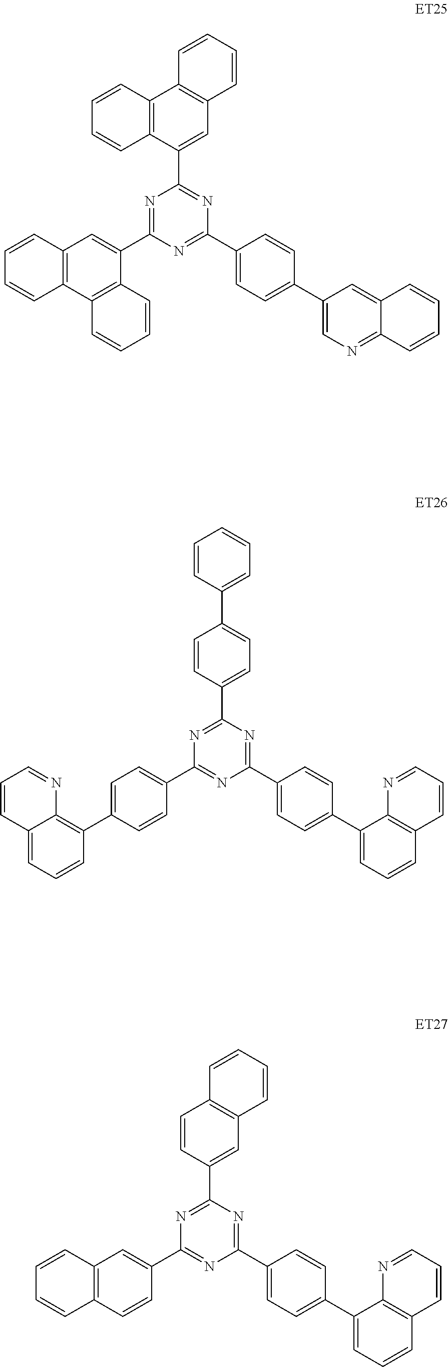 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

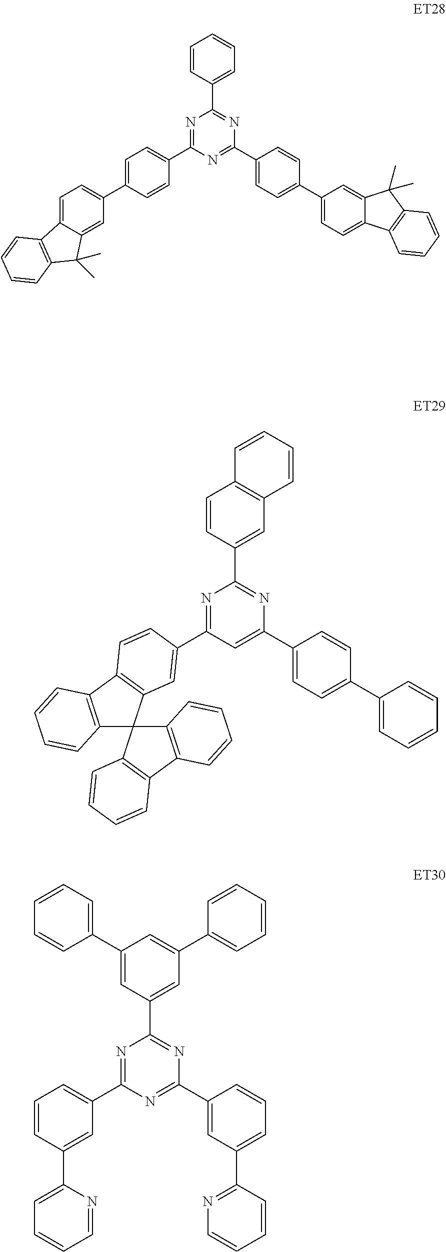 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

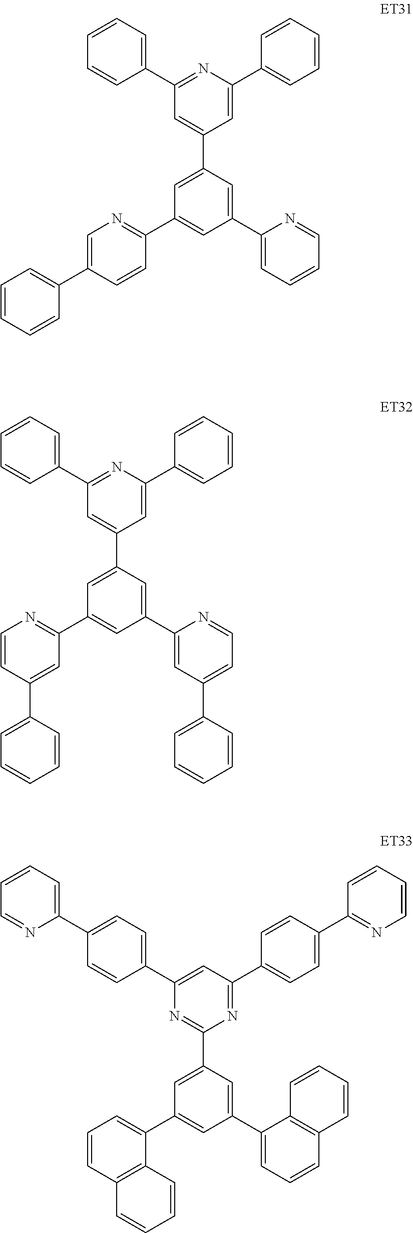 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

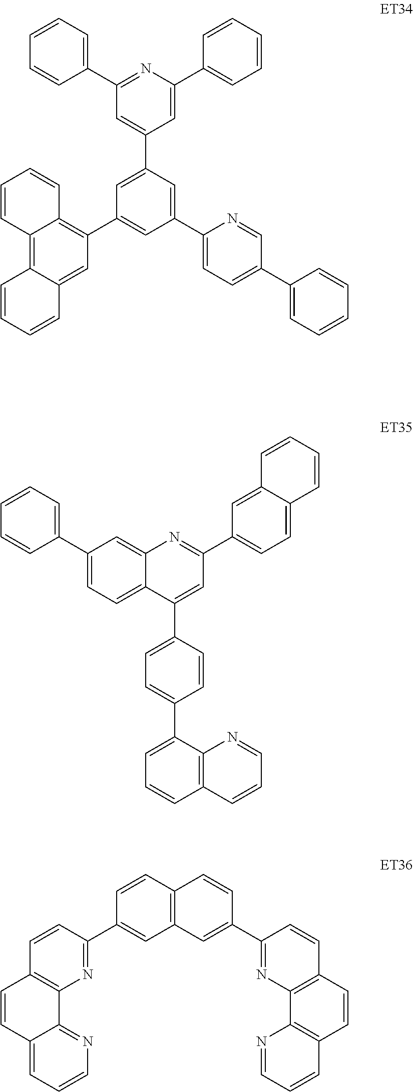 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

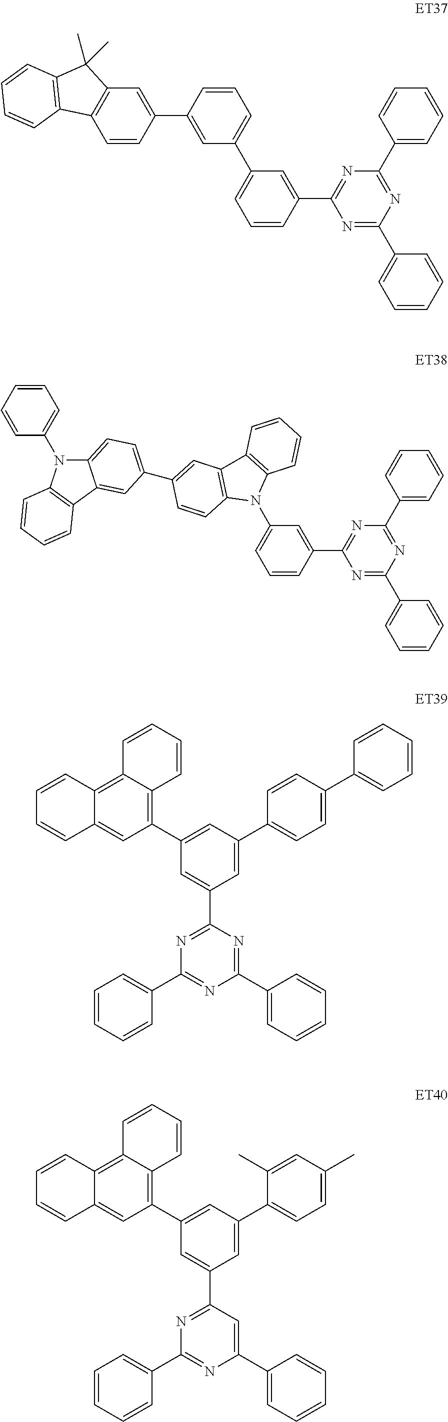 [SMILES: CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1 ; Cc1ccc(-c2cc(-c3cc(-c4ccccc4)nc(-c4ccccc4)n3)cc(-c3cc4ccccc4c4ccccc34)c2)c(C)c1]

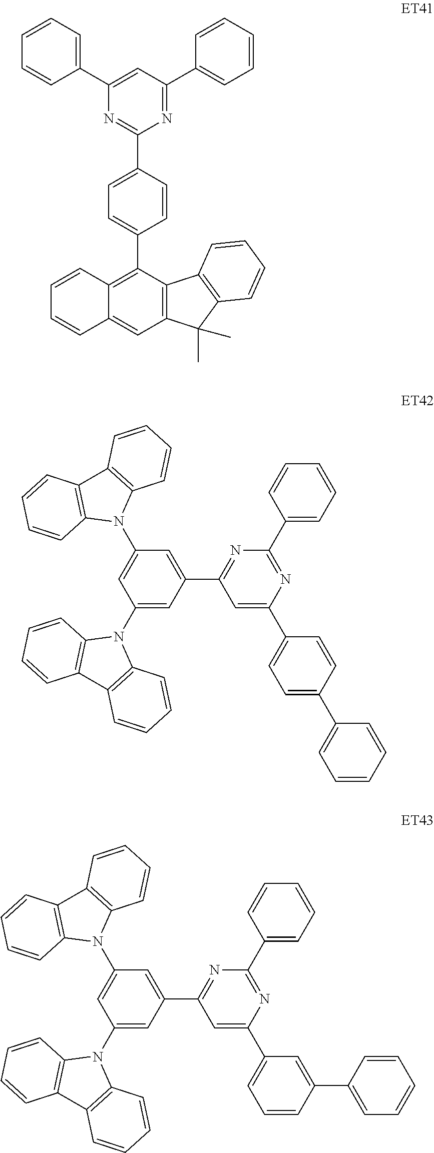 [SMILES: CC1(C)c2ccccc2-c2c1cc1ccccc1c2-c1ccc(-c2nc(-c3ccccc3)cc(-c3ccccc3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1]

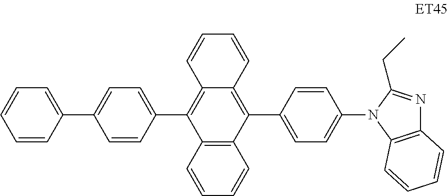 [SMILES: CCc1nc2ccccc2n1-c1ccc(-c2c3ccccc3c(-c3ccc(-c4ccccc4)cc3)c3ccccc23)cc1]

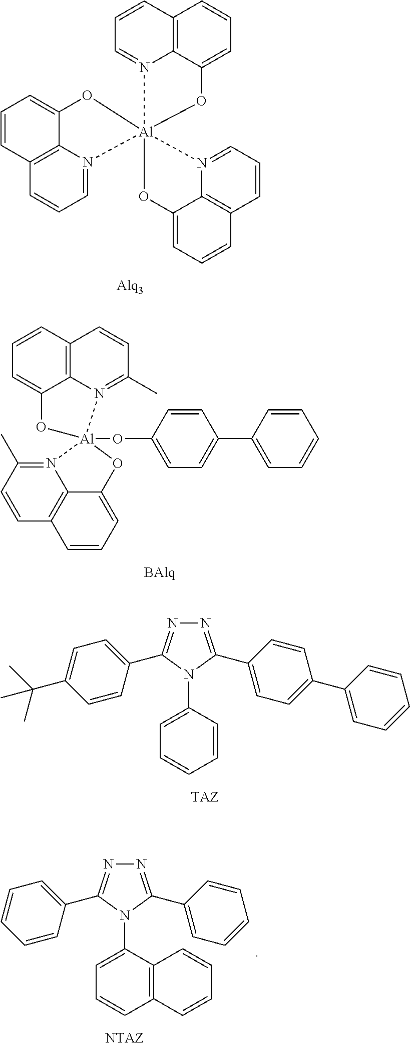

A thickness of the electron transport region 140 may be in a range of about 100 Å to about 5,000 Å, for example, about 160 Å to about 4,000 Å. When the electron transport region 140 includes a buffer layer, a hole blocking layer, an electron control layer, an electron transport layer, or any combination thereof, a thickness of the buffer layer, the hole blocking layer, or the electron control layer may each independently be in a range of about 20 Å to about 1,000 Å, for example, about 30 Å to about 300 Å, and a thickness of the electron transport layer may be in a range of about 100 Å to about 1,000 Å, for example, about 150 Å to about 500 Å. When the thicknesses of the buffer layer, the hole blocking layer, the electron control layer, the electron transport layer, and/or the electron transport region 140 are within these ranges, suitable or satisfactory electron transporting characteristics may be obtained without a substantial increase in driving voltage.

The electron transport region 140 (e.g., the electron transport layer in the electron transport region 140) may further include, in addition to the materials described above, a metal-containing material.

The metal-containing material may include an alkali metal complex, an alkaline earth metal complex, or any combination thereof. A metal ion of the alkali metal complex may be a Li ion, a Na ion, a K ion, a Rb ion, and/or a Cs ion, and a metal ion of the alkaline earth metal complex may be a Be ion, a Mg ion, a Ca ion, a Sr ion, and/or a Ba ion. A ligand coordinated with the metal ion of the alkali metal complex or the alkaline earth-metal complex may include hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenylbenzimidazole, hydroxyphenylbenzothiazole, bipyridine, phenanthroline, cyclopentadiene, or any combination thereof.

For example, the metal-containing material may include a Li complex. The Li complex may include, for example, Compound ET-D1 (Liq) and/or ET-D2:

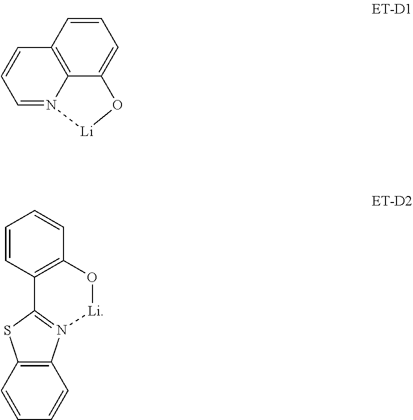

The electron transport region 140 may include an electron injection layer that facilitates the injection of electrons from the second electrode 150. The electron injection layer may be in direct contact with the second electrode 150.

The electron injection layer may have i) a single-layered structure consisting of a single layer consisting of a single material, ii) a single-layered structure consisting of a single layer including a plurality of different materials, or iii) a multi-layered structure including a plurality of layers including different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may include oxides, halides (e.g., fluorides, chlorides, bromides, iodides, etc.), and/or tellurides of the alkali metal, the alkaline earth metal, and/or the rare earth metal, or any combination thereof.

The alkali metal-containing compound may include an alkali metal oxide, such as Li₂O, Cs₂O, and/or K₂O, an alkali metal halide, such as LiF, NaF, CsF, KF, LiI, NaI, CsI, and/or KI, or any combination thereof. The alkaline earth metal-containing compound may include an alkaline earth metal compound (e.g., oxide), such as BaO, SrO, CaO, BaₓSr₁₋ₓO (wherein x is a real number satisfying the condition of 0<x<1), and/or BaₓCa₁₋ₓO (wherein x is a real number satisfying the condition of 0<x<1). The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In one or more embodiments, the rare earth metal-containing compound may include lanthanide metal telluride. Examples of the lanthanide metal telluride may include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, Lu₂Te₃, and/or the like.

The alkali metal complex, the alkaline earth-metal complex, and the rare earth metal complex may include i) one selected from ions of the alkali metal, the alkaline earth metal, and the rare earth metal and ii), as a ligand linked to the metal ion, for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or any combination thereof.

The electron injection layer may consist of (e.g., include) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In one or more embodiments, the electron injection layer may further include an organic material (e.g., a compound represented by Formula 601).

In one or more embodiments, the electron injection layer may consist of i) an alkali metal-containing compound (e.g., alkali metal halide), or ii) a) an alkali metal-containing compound (e.g., alkali metal halide); and b) an alkali metal, an alkaline earth metal, a rare earth metal, or any combination thereof. For example, the electron injection layer may be a KI:Yb co-deposited layer, a RbI:Yb co-deposited layer, a LiF:Yb co-deposited layer, and/or the like.

When the electron injection layer further includes an organic material, an alkali metal, an alkaline earth metal, a rare earth metal, an alkali metal-containing compound, an alkaline earth metal-containing compound, a rare earth metal-containing compound, an alkali metal complex, an alkaline earth-metal complex, a rare earth metal complex, or any combination thereof may be uniformly or non-uniformly dispersed in a matrix including the organic material.

A thickness of the electron injection layer may be in a range of about 1 Å to about 100 Å, for example, about 3 Å to about 90 Å. When the thickness of the electron injection layer is within these ranges, suitable or satisfactory electron injection characteristics may be obtained without a substantial increase in driving voltage.

### Second Electrode 150

The second electrode 150 may be arranged on the electron transport region 140. The second electrode 150 may be a cathode, which is an electron injection electrode, and as a material for forming the second electrode 150, a metal, an alloy, an electrically conductive compound, or any combination thereof, each having a low work function, may be used.

The second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a semi-transmissive electrode, or a reflective electrode.

The second electrode 150 may have a single-layered structure or a multi-layered structure including a plurality of layers.

### Capping Layer

A first capping layer may be arranged outside the first electrode 110, and/or a second capping layer may be arranged outside the second electrode 150. In more detail, the light-emitting device 10 may have a structure in which the first capping layer, the first electrode 110, the emission layer 130, and the second electrode 150 are sequentially stacked in the stated order, a structure in which the first electrode 110, the emission layer 130, the second electrode 150, and the second capping layer are sequentially stacked in the stated order, or a structure in which the first capping layer, the first electrode 110, the emission layer 130, the second electrode 150, and the second capping layer are sequentially stacked in the stated order.

In one or more embodiments, light generated in the emission layer 130 of the light-emitting device 10 may be extracted toward the outside through the first electrode 110, which is a semi-transmissive electrode or a transmissive electrode, and the first capping layer. In one or more embodiments, light generated in the emission layer 130 of the light-emitting device 10 may be extracted toward the outside through the second electrode 150, which is a semi-transmissive electrode or a transmissive electrode, and the second capping layer.

The first capping layer and the second capping layer may increase external luminescence efficiency according to the principle of constructive interference. Accordingly, the light extraction efficiency of the light-emitting device 10 may be increased, so that the luminescence efficiency of the light-emitting device 10 may be improved.

Each of the first capping layer and the second capping layer may include a material having a refractive index of 1.6 or more (at a wavelength of 589 nm).

The first capping layer and the second capping layer may each independently be an organic capping layer including an organic material, an inorganic capping layer including an inorganic material, or an organic-inorganic composite capping layer including an organic material and an inorganic material.

At least one selected from the first capping layer and the second capping layer may each independently include a carbocyclic compound, a heterocyclic compound, an amine group-containing compound, a porphine derivative, a phthalocyanine derivative, a naphthalocyanine derivative, an alkali metal complex, an alkaline earth metal complex, or any combination thereof. The carbocyclic compound, the heterocyclic compound, and the amine group-containing compound may optionally be substituted with a substituent including O, N, S, Se, Si, F, Cl, Br, I, or any combination thereof. In one or more embodiments, at least one selected from the first capping layer and the second capping layer may each independently include an amine group-containing compound.

For example, at least one selected from the first capping layer and the second capping layer may each independently include a compound represented by Formula 201, a compound represented by Formula 202, or any combination thereof.

In one or more embodiments, at least one selected from the first capping layer and the second capping layer may each independently include one selected from Compounds HT28 to HT33, one selected from Compounds CP1 to CP6, p-NPB, or any combination thereof:

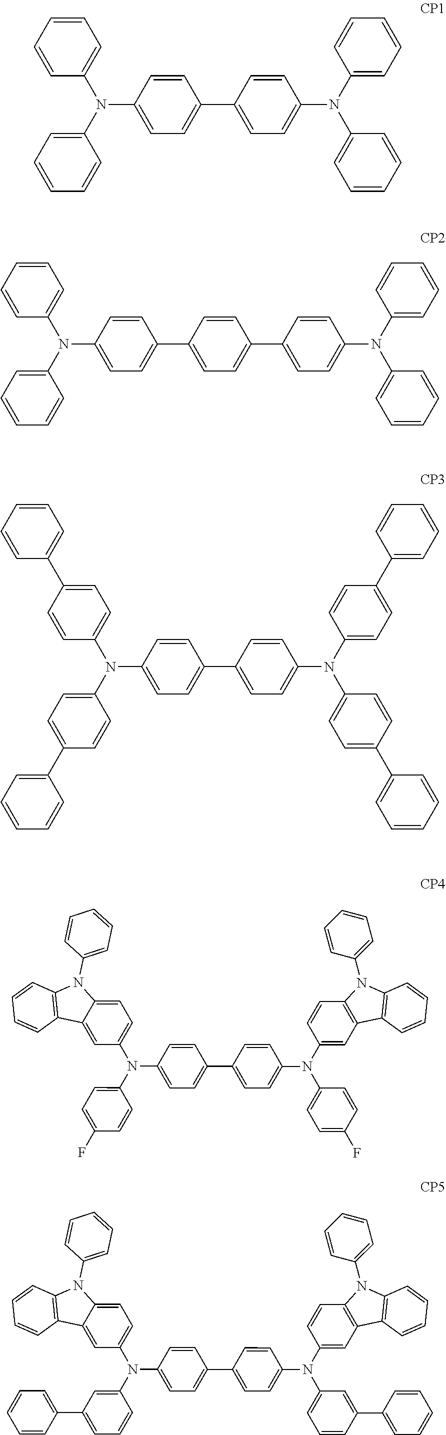 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1]

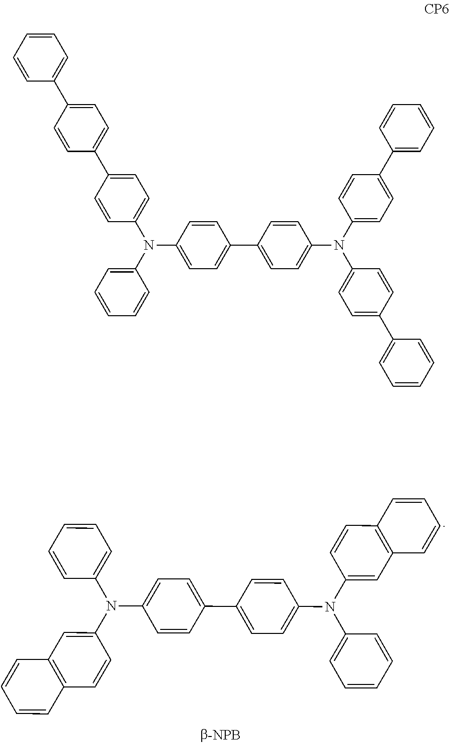 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

### Film

A film may be, for example, an optical member (or a light control member) (e.g., a color filter, a color conversion member, a capping layer, a light extraction efficiency improvement layer, a selective light absorption layer, a polarizing layer, a quantum dot-containing layer, etc.), a light blocking member (e.g., a light reflection layer, a light absorption layer, etc.), a protection member (e.g., an insulating layer, a dielectric layer, etc.), and/or the like.

### Electronic Apparatus

The light-emitting device 10 and the opto-electronic device 30 may be included in various suitable electronic apparatuses. For example, the electronic apparatus including the light-emitting device 10 and the opto-electronic device 30 may be a light-emitting apparatus, an authentication apparatus, and/or the like.

The electronic apparatus (e.g., a light-emitting apparatus) may further include, in addition to the light-emitting device 10 and the opto-electronic device 30, i) a color filter, ii) a color conversion layer, or iii) a color filter and a color conversion layer. The color filter and/or the color conversion layer may be arranged in at least one direction in which light emitted from the light-emitting device 10 travels. For example, the light emitted from the light-emitting device 10 may be blue light or white light. Further details of the light-emitting device 10 may be as described herein. In one or more embodiments, the color conversion layer may include a quantum dot. The quantum dot may be, for example, a quantum dot as described herein.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel defining layer may be arranged among the subpixel areas to define each of the subpixel areas.

The color filter may further include a plurality of color filter areas and light-shielding patterns arranged among the color filter areas, and the color conversion layer may further include a plurality of color conversion areas and light-shielding patterns arranged among the color conversion areas.

The color filter areas (or the color conversion areas) may include a first area that emits a first color light, a second area that emits a second color light, and/or a third area that emits a third color light, and the first color light, the second color light, and/or the third color light may have different maximum emission wavelengths. For example, the first color light may be red light, the second color light may be green light, and the third color light may be blue light. For example, the color filter areas (or the color conversion areas) may include quantum dots. In more detail, the first area may include a red quantum dot, the second area may include a green quantum dot, and the third area may not include a quantum dot. Further details of the quantum dot may be the same as described herein. The first area, the second area, and/or the third area may each further include a scatterer (e.g., a light scatterer).

For example, the light-emitting device 10 may emit a first light, the first area may absorb the first light to emit a first-first color light, the second area may absorb the first light to emit a second-first color light, and the third area may absorb the first light to emit a third-first color light. In this regard, the first-first color light, the second-first color light, and the third-first color light may have different maximum emission wavelengths. In more detail, the first light may be blue light, the first-first color light may be red light, the second-first color light may be green light, and the third-first color light may be blue light.

The electronic apparatus may further include a thin-film transistor, in addition to the light-emitting device 10 as described above. The thin-film transistor may include a source electrode, a drain electrode, and an active layer, and one selected from the source electrode and the drain electrode may be electrically connected to one selected from the first electrode 110 and the second electrode 150 of the light-emitting device 10.

The thin-film transistor may further include a gate electrode, a gate insulating film, and/or the like.

The active layer may include crystalline silicon, amorphous silicon, an organic semiconductor, an oxide semiconductor, and/or the like.

The electronic apparatus may further include a sealing portion for sealing the light-emitting device 10. The sealing portion may be arranged between the color filter and/or the color conversion layer and the light-emitting device 10. The sealing portion may allow light from the light-emitting device 10 to be extracted to the outside, and may concurrently (e.g., simultaneously) prevent or reduce penetration of ambient air and/or moisture into the light-emitting device 10. The sealing portion may be a sealing substrate including a transparent glass substrate and/or a plastic substrate. The sealing portion may be a thin-film encapsulation layer including at least one layer of an organic layer and/or an inorganic layer. When the sealing portion is a thin-film encapsulation layer, the electronic apparatus may be flexible.

Various suitable functional layers may be additionally arranged on the sealing portion, in addition to the color filter and/or the color conversion layer, according to the use of the electronic apparatus. Examples of the functional layers may include a touch screen layer, a polarizing layer, and/or the like. The touch screen layer may be a pressure-sensitive touch screen layer, a capacitive touch screen layer, and/or an infrared touch screen layer. The authentication apparatus may be, for example, a biometric authentication apparatus that authenticates an individual by using biometric information of a living body (e.g., fingertips, pupils, etc.).

The authentication apparatus may further include, in addition to the light-emitting device 10 as described above, a biometric information collector.

The electronic apparatus may be applied to various suitable displays, light sources, lighting, personal computers (e.g., mobile personal computers), mobile phones, digital cameras, electronic organizers, electronic dictionaries, electronic game machines, medical instruments (e.g., electronic thermometers, sphygmomanometers, blood glucose meters, pulse measurement devices, pulse wave measurement devices, electrocardiogram displays, ultrasonic diagnostic devices, and/or endoscope displays), fish finders, various suitable measuring instruments, meters (e.g., meters for a vehicle, an aircraft, and/or a vessel), projectors, and/or the like.

### Electronic Device

The light-emitting device 10 may be included in various suitable electronic devices.

For example, the electronic device including the light-emitting device 10 may be one selected from a flat panel display, a curved display, a computer monitor, a medical monitor, a television, a billboard, an indoor or outdoor lighting and/or signal light, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a microdisplay, a three-dimensional (3D) display, a virtual or augmented-reality display, a vehicle, a video wall including multiple displays tiled together, a theater or stadium screen, a phototherapy device, and a signboard.

The light-emitting device 10 may have excellent luminescence efficiency and long lifespan, and thus, the electronic device including the light-emitting device 10 may have characteristics such as high luminance, high resolution, and low power consumption.

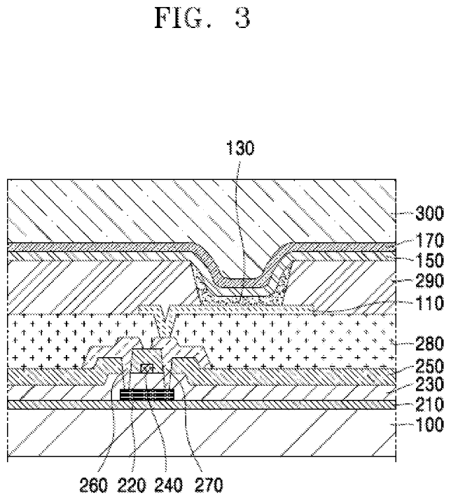

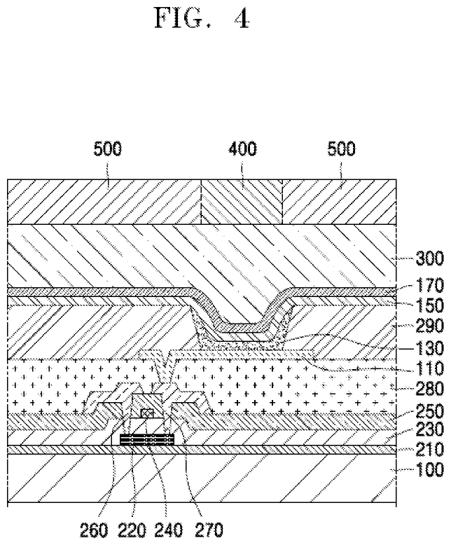

### Description of FIGS. 3 and 4

FIG. 3 is a cross-sectional view of an electronic apparatus according to one or more embodiments.

The electronic apparatus of FIG. 3 may include a substrate 100, a thin-film transistor (TFT), a light-emitting device, and an encapsulation portion 300 that seals the light-emitting device.

The substrate 100 may be a flexible substrate, a glass substrate, and/or a metal substrate. A buffer layer 210 may be arranged on the substrate 100. The buffer layer 210 may prevent or reduce penetration of impurities through the substrate 100 and may provide a flat surface on the substrate 100.

A TFT may be arranged on the buffer layer 210. The TFT may include an active layer 220, a gate electrode 240, a source electrode 260, and a drain electrode 270.

The active layer 220 may include an inorganic semiconductor, such as silicon or polysilicon, an organic semiconductor, or an oxide semiconductor, and may include a source region, a drain region, and a channel region.

A gate insulating film 230 for insulating the active layer 220 from the gate electrode 240 may be arranged on the active layer 220, and the gate electrode 240 may be arranged on the gate insulating film 230.

An interlayer insulating film 250 may be arranged on the gate electrode 240. The interlayer insulating film 250 may be arranged between the gate electrode 240 and the source electrode 260 to insulate the gate electrode 240 from the source electrode 260 and between the gate electrode 240 and the drain electrode 270 to insulate the gate electrode 240 from the drain electrode 270.

The source electrode 260 and the drain electrode 270 may be arranged on the interlayer insulating film 250. The interlayer insulating film 250 and the gate insulating film 230 may expose the source region and the drain region of the active layer 220, and the source electrode 260 and the drain electrode 270 may be in contact with the exposed portions of the source region and the drain region of the active layer 220.

The TFT may be electrically connected to a light-emitting device to drive the light-emitting device, and may be covered and protected by a passivation layer 280. The passivation layer 280 may include an inorganic insulating film, an organic insulating film, or a combination thereof. A light-emitting device may be provided on the passivation layer 280. The light-emitting device may include a first electrode 110, an emission layer 130, and a second electrode 150.

The first electrode 110 may be arranged on the passivation layer 280. The passivation layer 280 may not completely cover the drain electrode 270 and may expose a portion of the drain electrode 270, and the first electrode 110 may be connected to the exposed portion of the drain electrode 270.

A pixel defining layer 290 including an insulating material may be arranged on the first electrode 110. The pixel defining layer 290 may expose a portion of the first electrode 110, and the emission layer 130 may be formed in the exposed portion of the first electrode 110. The pixel defining layer 290 may be a polyimide and/or polyacrylic organic film. In one or more embodiments, at least some layers of the emission layer 130 may extend beyond the upper portion of the pixel defining layer 290 to be arranged in the form of a common layer.

The second electrode 150 may be arranged on the emission layer 130, and a capping layer 170 may be on the second electrode 150. The capping layer 170 may cover the second electrode 150.

The encapsulation portion 300 may be arranged on the capping layer 170. The encapsulation portion 300 may be arranged on a light-emitting device to protect the light-emitting device from moisture and/or oxygen. The encapsulation portion 300 may include: an inorganic film including silicon nitride (SiNₓ), silicon oxide (SiOₓ), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic-based resin (e.g., polymethyl methacrylate, polyacrylic acid, etc.), an epoxy-based resin (e.g., aliphatic glycidyl ether (AGE), etc.), or any combination thereof; or a combination of the inorganic film and the organic film.

FIG. 4 is a cross-sectional view of an electronic apparatus according to another embodiment.

The electronic apparatus of FIG. 4 is the same as the electronic apparatus of FIG. 3, except that a light-shielding pattern 500 and a functional region 400 are additionally arranged on the encapsulation portion 300. The functional region 400 may be i) a color filter area, ii) a color conversion area, or iii) a combination of the color filter area and the color conversion area. In one or more embodiments, the light-emitting device included in the electronic apparatus of FIG. 3 may be a tandem light-emitting device.

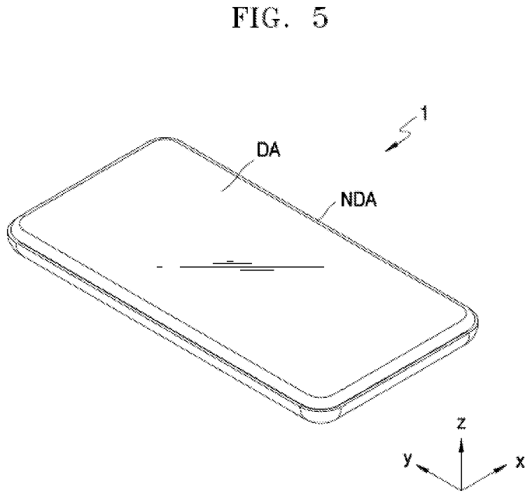

### Description of FIG. 5

FIG. 5 is a schematic perspective view of an electronic device 1 including a light-emitting device according to one or more embodiments. The electronic device 1 may be an apparatus that displays a moving image and/or a still image, and may include a portable electronic device, such as a mobile phone, a smart phone, a tablet personal computer (PC), a mobile communication terminal, an electronic notebook, an electronic book, a portable multimedia player (PMP), a navigation device, and/or an ultra mobile PC (UMPC), as well as various suitable products, such as a television, a laptop, a monitor, a billboard, and/or an internet of things (IOT) device, or a part thereof. In addition, the electronic device 1 may be a wearable device, such as a smart watch, a watch phone, a glasses-type display, and/or a head mounted display (HMD), or a part thereof. However, embodiments are not limited thereto. For example, the electronic device 1 may be an instrument panel of a vehicle, a center information display (CID) arranged on a center fascia and/or a dashboard of a vehicle, a room mirror display that replaces a side mirror of a vehicle, an entertainment display for a rear seat of a vehicle and/or a display arranged on a rear surface of a front seat, a head up display (HUD) installed at a front of a vehicle and/or projected on a front window glass, and/or a computer generated hologram augmented reality head up display (CGH AR HUD). For convenience of description, FIG. 5 shows a case in which the electronic device 1 is a smart phone.

The electronic device 1 may include a display area DA and a non-display area NDA outside the display area DA. The electronic device 1 may implement an image through an array of a plurality of pixels that are two-dimensionally arranged in the display area DA.

The non-display area NDA is an area in which an image is not displayed, and may entirely surround the display area DA. A driver for providing electrical signals or power to display elements arranged in the display area DA may be arranged in the non-display area NDA. A pad, which is an area to which an electronic element or a printed circuit board may be electrically connected, may be arranged in the non-display area NDA.

The electronic device 1 may have different lengths in the x-axis direction (or x-direction) and in the y-axis direction (or y-direction) and a thickness in the z-axis direction (or z-direction). For example, as shown in FIG. 5, the length in the x-axis direction may be shorter than the length in the y-axis direction. In one or more embodiments, the length in the x-axis direction may be the same as the length in the y-axis direction. In one or more embodiments, the length in the x-axis direction may be longer than the length in the y-axis direction.

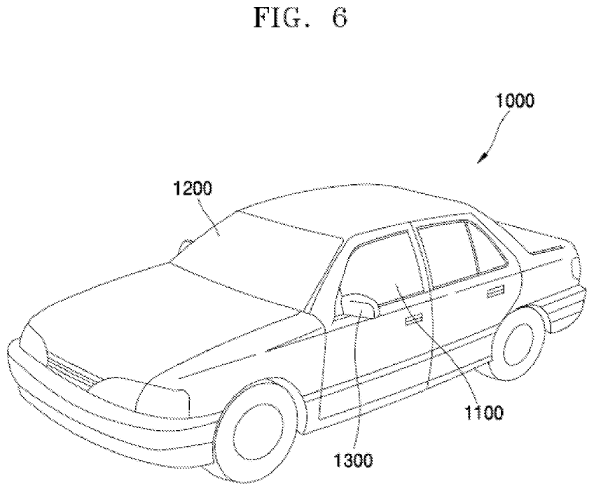

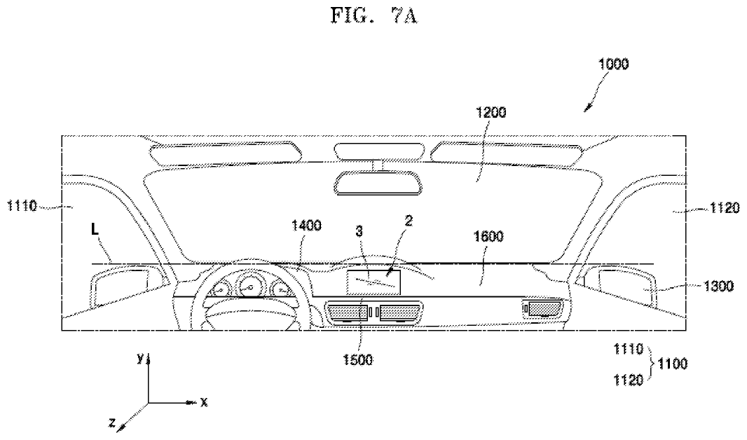

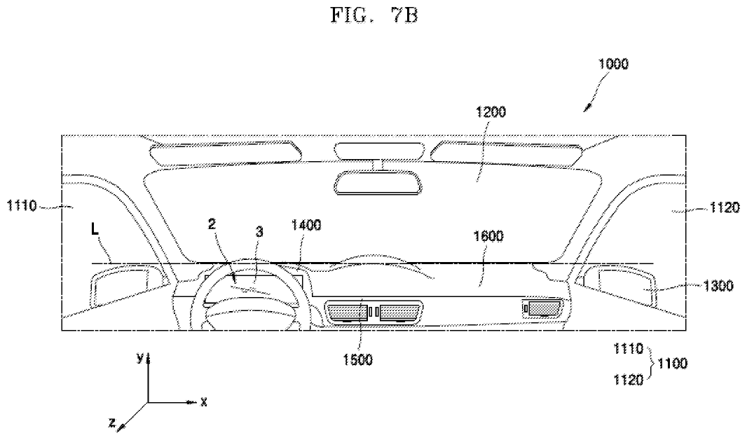

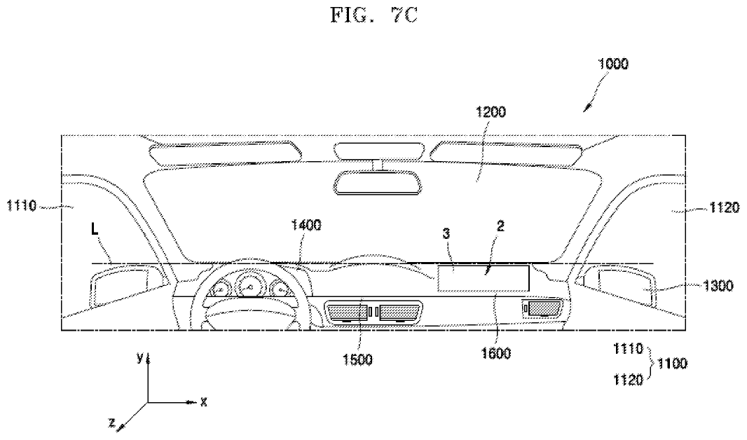

### Descriptions of FIGS. 6 and 7A to 7C

FIG. 6 is a schematic view of the exterior of a vehicle 1000 as an electronic device including a light-emitting device according to one or more embodiments. FIGS. 7A to 7C are schematic views of the interior of the vehicle 1000 according to various embodiments.

Referring to FIGS. 6, 7A, 7B, and 7C, the vehicle 1000 may refer to various suitable apparatuses for moving a subject to be transported, such as a human, an object, and/or an animal, from a departure point to a destination. The vehicle 1000 may include a vehicle traveling on a road and/or a track, a vessel moving over the sea and/or a river, an airplane flying in the sky by using the action of air, and the like.

The vehicle 1000 may travel on a road and/or a track. The vehicle 1000 may move in a certain direction according to rotation of at least one wheel. For example, the vehicle 1000 may include a three-wheeled or four-wheeled vehicle, a construction machine, a two-wheeled vehicle, a prime mover apparatus, a bicycle, and/or a train running on a track.

The vehicle 1000 may include a body having an interior and an exterior, and a chassis in which mechanical apparatuses useful or necessary for driving are installed as the remaining parts except for the body. The exterior of the body may include a front panel, a bonnet, a roof panel, a rear panel, a trunk, and/or a pillar provided at a boundary between doors. The chassis of the vehicle 1000 may include a power generating apparatus, a power transmitting apparatus, a driving apparatus, a steering apparatus, a braking apparatus, a suspension apparatus, a transmission apparatus, a fuel apparatus, front and rear wheels, left and right wheels, and/or the like.

The vehicle 1000 may include a side window glass 1100, a front window glass 1200, a side mirror 1300, a cluster 1400, a center fascia 1500, a passenger seat dashboard 1600, and/or a display apparatus 2.

The side window glass 1100 and the front window glass 1200 may be partitioned by a pillar arranged between the side window glass 1100 and the front window glass 1200.

The side window glass 1100 may be installed on the side of the vehicle 1000. In one or more embodiments, the side window glass 1100 may be installed on a door of the vehicle 1000. A plurality of side window glasses 1100 may be provided and may face each other. In one or more embodiments, the side window glass 1100 may include a first side window glass 1110 and a second side window glass 1120. In one or more embodiments, the first side window glass 1110 may be arranged adjacent to the cluster 1400. The second side window glass 1120 may be arranged adjacent to the passenger seat dashboard 1600.

In one or more embodiments, the side window glasses 1100 may be apart from each other in the x-direction or the direction opposite to the x-direction. For example, the first side window glass 1110 and the second side window glass 1120 may be apart from each other in the x direction or the direction opposite to the x direction. In other words, an imaginary straight line L connecting the side window glasses 1100 to each other may extend in the x direction or the direction opposite to the x direction. For example, the imaginary straight line L connecting the first side window glass 1110 and the second side window glass 1120 to each other may extend in the x direction or the direction opposite to the x direction.

The front window glass 1200 may be installed in the front of the vehicle 1000. The front window glass 1200 may be arranged between the side window glasses 1100 facing each other.

The side mirror 1300 may provide a view of the rear of the vehicle 1000. The side mirror 1300 may be installed on the exterior of the body. In one or more embodiments, a plurality of side mirrors 1300 may be provided. One of the plurality of side mirrors 1300 may be arranged outside the first side window glass 1110. Another one of the plurality of side mirrors 1300 may be arranged outside the second side window glass 1120.

The cluster 1400 may be arranged in front of the steering wheel. The cluster 1400 may include a tachometer, a speedometer, a coolant thermometer, a fuel gauge, a turn indicator, a high beam indicator, a warning lamp, a seat belt warning lamp, an odometer, a hodometer, an automatic transmission selection lever indicator, a door open warning lamp, an engine oil warning lamp, and/or a low fuel warning lamp.

The center fascia 1500 may include a control panel including a plurality of buttons for adjusting an audio apparatus, an air conditioning apparatus, and/or a heater of a seat. The center fascia 1500 may be arranged on one side of the cluster 1400.

The passenger seat dashboard 1600 may be apart from the cluster 1400 with the center fascia 1500 therebetween. In one or more embodiments, the cluster 1400 may be arranged to correspond to a driver seat, and the passenger seat dashboard 1600 may be arranged to correspond to a passenger seat. In one or more embodiments, the cluster 1400 may be adjacent to the first side window glass 1110, and the passenger seat dashboard 1600 may be adjacent to the second side window glass 1120.

In one or more embodiments, the display apparatus 2 may include a display panel 3, and the display panel 3 may display an image. The display apparatus 2 may be arranged inside the vehicle 1000. In one or more embodiments, the display apparatus 2 may be arranged between the side window glasses 1100 facing each other. The display apparatus 2 may be arranged on at least one selected from the cluster 1400, the center fascia 1500, and the passenger seat dashboard 1600.

The display apparatus 2 may include an organic light-emitting display apparatus, an inorganic electroluminescent (EL) display apparatus, a quantum dot display apparatus, and/or the like. Hereinafter, as the display apparatus 2 according to one or more embodiments, an organic light-emitting display apparatus including the light-emitting device according to one or more embodiments will be described as an example, but various suitable types or kinds of display apparatuses as described above may be used in embodiments.

Referring to FIG. 7A, the display apparatus 2 may be arranged on the center fascia 1500. In one or more embodiments, the display apparatus 2 may display navigation information. In one or more embodiments, the display apparatus 2 may display audio, video, and/or information regarding vehicle settings.

Referring to FIG. 7B, the display apparatus 2 may be arranged on the cluster 1400. In this case, the cluster 1400 may display driving information and/or the like through the display apparatus 2. For example, the cluster 1400 may be digitally implemented. The digital cluster 1400 may display vehicle information and/or driving information as images. For example, a needle and a gauge of a tachometer and/or various suitable warning lamp icons may be displayed by digital signals.

Referring to FIG. 7C, the display apparatus 2 may be arranged on the passenger seat dashboard 1600. The display apparatus 2 may be embedded in the passenger seat dashboard 1600 and/or arranged on the passenger seat dashboard 1600. In one or more embodiments, the display apparatus 2 arranged on the passenger seat dashboard 1600 may display an image related to information displayed on the cluster 1400 and/or information displayed on the center fascia 1500. In one or more embodiments, the display apparatus 2 arranged on the passenger seat dashboard 1600 may display information that is different from information displayed on the cluster 1400 and/or information displayed on the center fascia 1500.

### Manufacturing Method

Respective layers included in the hole transport region 120, the emission layer 130, the first and second layers 131 and 132 included in the photoactive layer 135, and respective layers included in the electron transport region 140 may be formed in a certain region by using one or more suitable methods selected from vacuum deposition, spin coating, casting, Langmuir-Blodgett (LB) method, ink-jet printing, laser-printing, and laser-induced thermal imaging (LITI).

When respective layers included in the hole transport region 120, the emission layer 130, the first and second layers 131 and 132 included in the photoactive layer 135, and respective layers included in the electron transport region 140 are formed by vacuum deposition, the vacuum deposition may be performed at a deposition temperature of about 100° C. to about 500° C., a vacuum degree of about 10⁻⁸ torr to about 10⁻³ torr, and a deposition speed of about 0.01 Å/sec to about 100 Å/sec, depending on a material to be included in a layer to be formed and the structure of a layer to be formed. Because the organic compound, in which a first moiety represented by Formula 1 and a second moiety represented by Formula 2 are bonded together, has excellent heat resistance, when the first layer 131 included in the photoactive layer 135 is formed using the organic compound by vacuum deposition, the first layer 131 may be easily formed without defects.

### Definition of Terms

The term “C₃-C₆₀ carbocyclic group” as used herein refers to a cyclic group consisting of carbon only as a ring-forming atom and having 3 to 60 carbon atoms, and the term “C₁-C₆₀ heterocyclic group” as used herein refers to a cyclic group that has 1 to 60 carbon atoms and further has, in addition to carbon, a heteroatom as a ring-forming atom. The C₃-C₆₀ carbocyclic group and the C₁-C₆₀ heterocyclic group may each be a monocyclic group consisting of one ring or a polycyclic group in which two or more rings are condensed together with each other. For example, the C₁-C₆₀ heterocyclic group may have 3 to 61 ring-forming atoms.

The “cyclic group” as used herein may include both the C₃-C₆₀ carbocyclic group and the C₁-C₆₀ heterocyclic group.

The term “π electron-rich C₃-C₆₀ cyclic group” as used herein refers to a cyclic group that has 3 to 60 carbon atoms and does not include *—N=*′ as a ring-forming moiety, and the term “π electron-deficient nitrogen-containing C₁-C₆₀ cyclic group” as used herein refers to a heterocyclic group that has 1 to 60 carbon atoms and includes *—N=*′ as a ring-forming moiety.

For example, the C₃-C₆₀ carbocyclic group may be i) a group T1 or ii) a condensed cyclic group in which two or more groups T1 are condensed together with each other (e.g., a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),

the C₁-C₆₀ heterocyclic group may be i) a group T2, ii) a condensed cyclic group in which two or more groups T2 are condensed together with each other, or iii) a condensed cyclic group in which at least one group T2 and at least one group T1 are condensed together with each other (e.g., a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),

the π electron-rich C₃-C₆₀ cyclic group may be i) a group T1, ii) a condensed cyclic group in which two or more groups T1 are condensed together with each other, iii) a group T3, iv) a condensed cyclic group in which two or more groups T3 are condensed together with each other, or v) a condensed cyclic group in which at least one group T3 and at least one group T1 are condensed together with each other (e.g., the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, etc.), and

the π electron-deficient nitrogen-containing C₁-C₆₀ cyclic group may be i) a group T4, ii) a condensed cyclic group in which two or more groups T4 are condensed together with each other, iii) a condensed cyclic group in which at least one group T4 and at least one group T1 are condensed together with each other, iv) a condensed cyclic group in which at least one group T4 and at least one group T3 are condensed together with each other, or v) a condensed cyclic group in which at least one group T4, at least one group T1, and at least one group T3 are condensed together with each other (e.g., a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),

wherein the group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or a bicyclo[2.2.1]heptane) group, a norbornene group, a bicyclo[1.1.1]pentane group, a bicyclo[2.1.1]hexane group, a bicyclo[2.2.2]octane group, or a benzene group,

the group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,

the group T3 may be a furan group, a thiophene group, a 1H-pyrrole group, a silole group, or a borole group, and

the group T4 may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The term “cyclic group,” “C₃-C₆₀ carbocyclic group,” “C₁-C₆₀ heterocyclic group,” “π electron-rich C₃-C₆₀ cyclic group,” or “π electron-deficient nitrogen-containing C₁-C₆₀ cyclic group” as used herein refers to a group condensed together with any cyclic group, a monovalent group, or a polyvalent group (e.g., a divalent group, a trivalent group, a tetravalent group, etc.), depending on the structure of a formula in connection with which the terms are used. For example, the “benzene group” may be a benzo group, a phenyl group, a phenylene group, or the like, which may be easily understood by one of ordinary skill in the art according to the structure of a formula including the “benzene group.”

Examples of the monovalent C₃-C₆₀ carbocyclic group and the monovalent C₁-C₆₀ heterocyclic group may include a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group, and examples of the divalent C₃-C₆₀ carbocyclic group and the divalent C₁-C₆₀ heterocyclic group may include a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a divalent non-aromatic condensed heteropolycyclic group.

The term “C₁-C₆₀ alkyl group” as used herein refers to a linear or branched aliphatic saturated hydrocarbon monovalent group that has 1 to 60 carbon atoms, and examples thereof may include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, a tert-decyl group, and the like. The term “C₁-C₆₀ alkylene group” as used herein refers to a divalent group having substantially the same structure as the C₁-C₆₀ alkyl group.

The term “C₂-C₆₀ alkenyl group” as used herein refers to a monovalent hydrocarbon group having at least one carbon-carbon double bond in the middle or at the terminus of the C₂-C₆₀ alkyl group, and examples thereof may include an ethenyl group, a propenyl group, a butenyl group, and the like. The term “C₂-C₆₀ alkenylene group” as used herein refers to a divalent group having substantially the same structure as the C₂-C₆₀ alkenyl group.

The term “C₂-C₆₀ alkynyl group” as used herein refers to a monovalent hydrocarbon group having at least one carbon-carbon triple bond in the middle or at the terminus of the C₂-C₆₀ alkyl group, and examples thereof may include an ethynyl group, a propynyl group, and the like. The term “C₂-C₆₀ alkynylene group” as used herein refers to a divalent group having substantially the same structure as the C₂-C₆₀ alkynyl group.

The term “C₁-C₆₀ alkoxy group” as used herein refers to a monovalent group represented by —OA₁₀₁ (wherein A₁₀₁ is the C₁-C₆₀ alkyl group), and examples thereof may include a methoxy group, an ethoxy group, an isopropyloxy group, and the like.

The term “C₃-C₁₀ cycloalkyl group” as used herein refers to a monovalent saturated hydrocarbon cyclic group having 3 to 10 carbon atoms, and examples thereof may include a cyclopropyl group, a cyclobutyl group, a cyclopentyl group, a cyclohexyl group, a cycloheptyl group, a cyclooctyl group, an adamantanyl group, a norbornanyl group (or a bicyclo[2.2.1]heptyl group), a bicyclo[1.1.1]pentyl group, a bicyclo[2.1.1]hexyl group, a bicyclo[2.2.2]octyl group, and the like. The term “C₃-C₁₀ cycloalkylene group” as used herein refers to a divalent group having substantially the same structure as the C₃-C₁₀ cycloalkyl group.

The term “C₁-C₁₀ heterocycloalkyl group” as used herein refers to a monovalent cyclic group that has 1 to 10 carbon atoms and further includes, in addition to carbon atoms, at least one heteroatom as a ring-forming atom, and examples thereof may include a 1,2,3,4-oxatriazolidinyl group, a tetrahydrofuranyl group, a tetrahydrothiophenyl group, and the like. The term “C₁-C₁₀ heterocycloalkylene group” as used herein refers to a divalent group having substantially the same structure as the C₁-C₁₀ heterocycloalkyl group.

The term “C₃-C₁₀ cycloalkenyl group” as used herein refers to a monovalent cyclic group that has 3 to 10 carbon atoms and at least one carbon-carbon double bond in the ring thereof and no aromaticity (e.g., is not aromatic), and examples thereof may include a cyclopentenyl group, a cyclohexenyl group, a cycloheptenyl group, and the like. The term “C₃-C₁₀ cycloalkenylene group” as used herein refers to a divalent group having substantially the same structure as the C₃-C₁₀ cycloalkenyl group.

The term “C₁-C₁₀ heterocycloalkenyl group” as used herein refers to a monovalent cyclic group that has 1 to 10 carbon atoms, at least one heteroatom as a ring-forming atom, in addition to carbon atoms, and at least one double bond in the ring thereof. Examples of the C₁-C₁₀ heterocycloalkenyl group may include a 4,5-dihydro-1,2,3,4-oxatriazolyl group, a 2,3-dihydrofuranyl group, a 2,3-dihydrothiophenyl group, and the like. The term “C₁-C₁₀ heterocycloalkenylene group” as used herein refers to a divalent group having substantially the same structure as the C₁-C₁₀ heterocycloalkenyl group.

The term “C₆-C₆₀ aryl group” as used herein refers to a monovalent group having a carbocyclic aromatic system of 6 to 60 carbon atoms, and the term “C₆-C₆₀ arylene group” as used herein refers to a divalent group having a carbocyclic aromatic system of 6 to 60 carbon atoms. Examples of the C₆-C₆₀ aryl group may include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, an ovalenyl group, and the like. When the C₆-C₆₀ aryl group and the C₆-C₆₀ arylene group each include two or more rings, the two or more rings may be condensed together with each other.

The term “C₁-C₆₀ heteroaryl group” as used herein refers to a monovalent group having a heterocyclic aromatic system of 1 to 60 carbon atoms, and further including, in addition to carbon atoms, at least one heteroatom, as ring-forming atoms, and the term “C₁-C₆₀ heteroarylene group” as used herein refers to a divalent group having a heterocyclic aromatic system of 1 to 60 carbon atoms, and further including, in addition to carbon atoms, at least one heteroatom, as ring-forming atoms. Examples of the C₁-C₆₀ heteroaryl group may include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, a naphthyridinyl group, and the like. When the C₁-C₆₀ heteroaryl group and the C₁-C₆₀ heteroarylene group each include two or more rings, the two or more rings may be condensed together with each other.

The term “monovalent non-aromatic condensed polycyclic group” as used herein refers to a monovalent group that has two or more rings condensed together with each other, only carbon atoms (e.g., 8 to 60 carbon atoms) as ring-forming atoms, and no aromaticity in its entire molecular structure (e.g., is not aromatic). Examples of the monovalent non-aromatic condensed polycyclic group may include an indenyl group, a fluorenyl group, a spiro-bifluorenyl group, a benzofluorenyl group, an indenophenanthrenyl group, an indenoanthracenyl group, and the like. The term “divalent non-aromatic condensed polycyclic group” as used herein refers to a divalent group having substantially the same structure as the monovalent non-aromatic condensed polycyclic group.

The term “monovalent non-aromatic condensed heteropolycyclic group” as used herein refers to a monovalent group that has two or more rings condensed together with each other, at least one heteroatom other than carbon atoms (e.g., 1 to 60 carbon atoms), as a ring-forming atom, and no aromaticity in its entire molecular structure (e.g., is not aromatic). Examples of the monovalent non-aromatic condensed heteropolycyclic group may include a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indenocarbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, a benzothienodibenzothiophenyl group, and the like. The term “divalent non-aromatic condensed heteropolycyclic group” as used herein refers to a divalent group having substantially the same structure as the monovalent non-aromatic condensed heteropolycyclic group.

The term “C₆-C₆₀ aryloxy group” as used herein refers to —OA₁₀₂ (wherein A₁₀₂ is the C₆-C₆₀ aryl group), and the term “C₆-C₆₀ arylthio group” as used herein refers to —SA₁₀₃ (wherein A₁₀₃ is the C₆-C₆₀ aryl group).

The term “C₇-C₆₀ arylalkyl group” as used herein refers to -A₁₀₄A₁₀₅ (wherein A₁₀₄ is a C₁-C₅₄ alkylene group, and A₁₀₅ is a C₆-C₅₉ aryl group), and the term “C₂-C₆₀ heteroarylalkyl group” as used herein refers to -A₁₀₆A₁₀₇ (wherein A₁₀₆ is a C₁-C₅₉ alkylene group, and A₁₀₇ is a C₁-C₅₉ heteroaryl group).

The term “R₁₀ₐ” as used herein refers to:

deuterium, —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;

a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

Q₁ to Q₃, Q₁₁ to Q₁₃, Q₂₁ to Q₂₃, and Q₃₁ to Q₃₃ as used herein may each independently be: hydrogen; deuterium; —F; —CI; —Br; —I; a hydroxyl group; a cyano group; a nitro group; a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, a hydroxyl group, a cyano group, a nitro group, or any combination thereof; a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a C₃-C₆₀ carbocyclic group, a C₁-C₆₀ heterocyclic group, or any combination thereof; a C₇-C₆₀ arylalkyl group; or a C₂-C₆₀ heteroarylalkyl group.

The term “heteroatom” as used herein refers to any suitable atom other than a carbon atom. Examples of the heteroatom may include O, S, N, P, Si, B, Ge, Se, or any combination thereof.

The term “third-row transition metal” as used herein may include hafnium (Hf), tantalum (Ta), tungsten (W), rhenium (Re), osmium (Os), iridium (Ir), platinum (Pt), gold (Au), and the like.

The term “Ph” as used herein refers to a phenyl group, the term “Me” as used herein refers to a methyl group, the term “Et” as used herein refers to an ethyl group, the term “tert-Bu” or “Bu^{t}” as used herein refers to a tert-butyl group, and the term “OMe” as used herein refers to a methoxy group.

The term “biphenyl group” as used herein refers to “a phenyl group substituted with a phenyl group.” In other words, the “biphenyl group” is a substituted phenyl group having a C₆-C₆₀ aryl group as a substituent.

The term “terphenyl group” as used herein refers to “a phenyl group substituted with a biphenyl group.” In other words, the “terphenyl group” is a substituted phenyl group having, as a substituent, a C₆-C₆₀ aryl group substituted with a C₆-C₆₀ aryl group.

* and *′ as used herein, unless defined otherwise, each refer to a binding site to a neighboring atom in a corresponding formula or moiety.

The x-axis, y-axis, and z-axis as used herein are not limited to three axes in an orthogonal coordinate system, and may be interpreted in a broad sense including these axes. For example, the x-axis, y-axis, and z-axis may refer to those orthogonal to each other, or may refer to those in different directions that are not orthogonal to each other.

Hereinafter, organic compounds according to embodiments and light-emitting devices and opto-electronic devices according to embodiments will be described in more detail with reference to Synthesis Examples and Examples. The wording “B was used instead of A” used in describing Synthesis Examples means that an identical molar equivalent of B was used in place of an identical molar equivalent of A.

### Synthesis Example 1 (Synthesis of Compound 4)

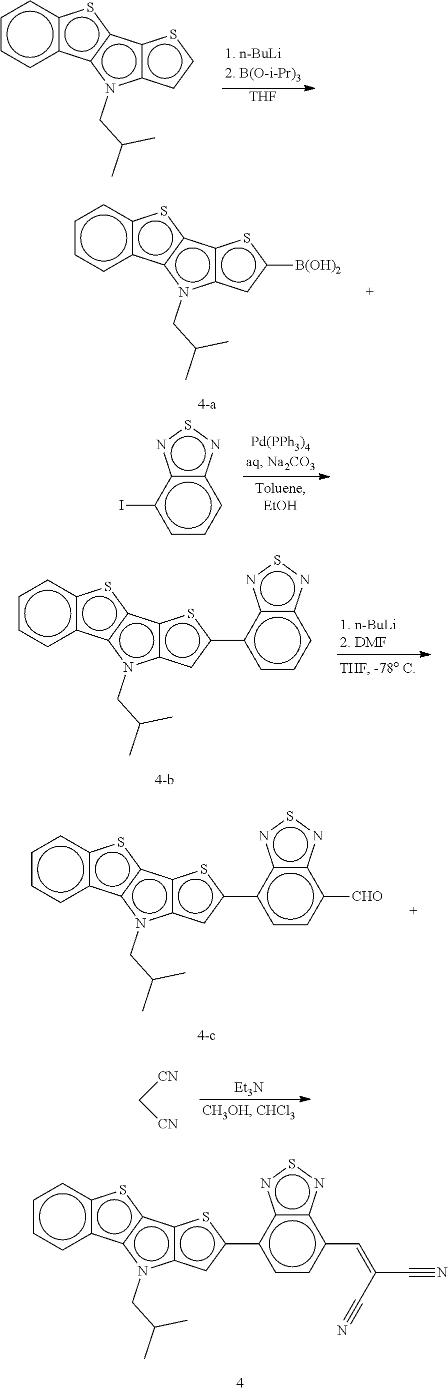

### Synthesis of Intermediate 4-a

In an argon atmosphere, 4-isobutyl-4H-benzo[4,5]thieno[3,2-b]thieno[2,3-d]pyrrole (11.4 mL, 40 mmol) was dissolved in 40 mL of tetrahydrofuran (THF), and the resultant mixture was cooled to −78° C. n-BuLi (27.5 mL, 1.6M in hexanes, 44 mmol) was added dropwise thereto for 10 minutes, and the mixture was stirred for 40 minutes. Then, the mixture was added dropwise to 20 mL of a THF solution of B(O-i-Pr)₃ (13.9 mL, 60 mmol), which had been cooled to 0° C., for 15 minutes. The mixture was stirred at the same temperature for 1 hour and then at room temperature for 17 hours. After completion of the reaction, the solvent was removed therefrom under reduced pressure, and the reaction product was separated and purified by column chromatography using ethyl acetate and hexane to obtain Intermediate 4-a (6.6 g, 70%).

The obtained compound was identified by electro-spray ionization liquid chromatography-mass spectrometry (ESI-LCMS) as Intermediate 4-a. (ESI-LCMS: [M]⁺: C₁₆H₁₆BNO₂S₂. 329.07)

### Synthesis of Intermediate 4-b

In an argon atmosphere, 4 mL of an ethanol (EtOH) solution of Intermediate 4-a (2.6 g, 8.0 mmol) and 3.6 mL of a 2M aqueous sodium carbonate (Na₂CO₃) solution were added to 10 mL of a toluene solution of 4-iodobenzo[c][1,2,5]thiadiazole (1.0 g, 4.0 mmol), and the resultant mixture was purged with nitrogen while being degassed. Then, Pd(PPh₃)₄ (0.3 g, 0.3 mmol) was added thereto, and the mixture was stirred at 80° C. for 12 hours. After cooling, an extraction process was performed thereon using water (500 mL) and ethyl acetate (300 mL) to collect an organic layer, which was then dried using anhydrous MgSO₄ and filtered. The filtered solution was placed under reduced pressure to remove the solvent therefrom, and the obtained solid was separated and purified by column chromatography using CH₂Cl₂ and hexane to obtain Intermediate 4-b (1.0 g, 2.5 mmol, 60%).

The obtained compound was identified by ESI-LCMS as Intermediate 4-b. (ESI-LCMS: [M]⁺: C₂₂H₁₇N₃S₃. 419.06)

### Synthesis of Intermediate 4-c

In an argon atmosphere, Intermediate 4-b (1.0 g, 2.3 mmol) was dissolved in 50 mL of THF, and the resultant mixture was cooled to −78° C. n-BuLi (1.9 mL, 1.6M in hexanes, 3.0 mmol) was added thereto, and the mixture was stirred for 30 minutes. Then, dimethylformamide (DMF) (0.2 mL, 3.0 mmol) was added thereto, and the mixture was stirred at the same temperature for 30 minutes and then at room temperature for 10 hours. After cooling, an extraction process was performed thereon using water (500 mL) and ethyl acetate (300 mL) to collect an organic layer, which was then dried using anhydrous MgSO₄ and filtered. The filtered solution was placed under reduced pressure to remove the solvent therefrom, and the obtained solid was separated and purified by column chromatography using CH₂Cl₂ and hexane to obtain Intermediate 4-c (0.6 g, 1.3 mmol, 58%).

The obtained compound was identified by ESI-LCMS as Intermediate 4-c. (ESI-LCMS: [M]⁺: C₂₃H₁₇N₃OS₃. 447.05)

### Synthesis of Compound 4

In an argon atmosphere, Intermediate 4-c (0.18 g, 0.4 mmol) was dissolved in 5 mL of chloroform (CHCl₃), malononitrile (0.03 g, 0.5 mmol), 5 mL of a methanol (CH₃OH) solution, and 1 mL of Et₃N were added thereto, and the resultant mixture was stirred at room temperature for 35 minutes and then concentrated under reduced pressure. The obtained solid was separated and purified by column chromatography using CH₂Cl₂ and hexane to obtain Compound 4 (0.13 g, 0.27 mmol, 61%).

The obtained compound was identified by ESI-LCMS as Compound 4. (ESI-LCMS: [M]⁺: C₂₆H₁₇N₅S₃. 495.06)

### Synthesis Example 2 (Synthesis of Compound 7)

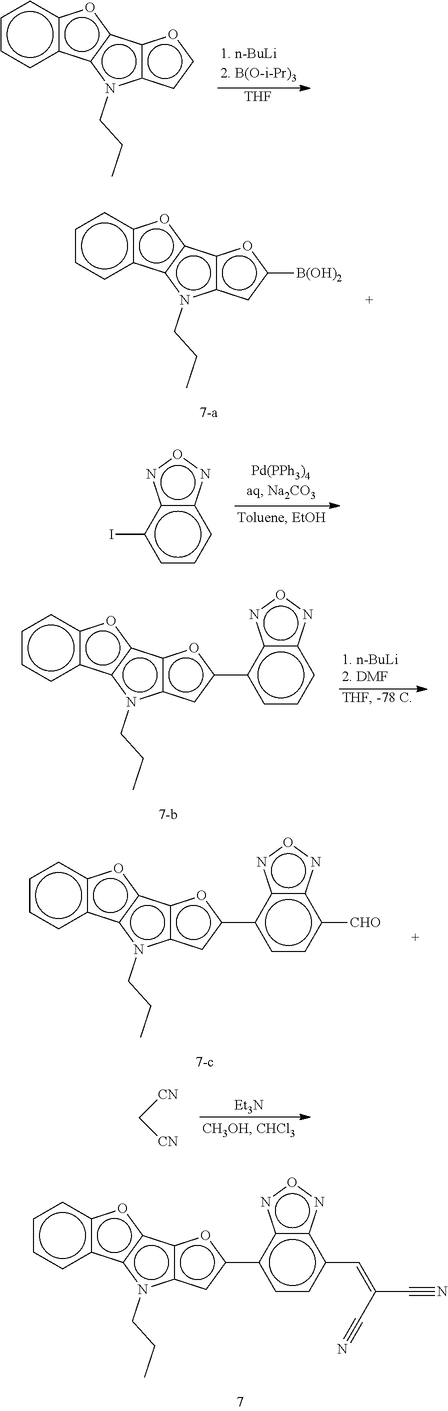

### Synthesis of Intermediate 7-a

In an argon atmosphere, 4-propyl-4H-benzofuro[3,2-b]furo[2,3-d]pyrrole (9.6 mL, 40 mmol) was dissolved in 40 mL of THF, and the resultant mixture was cooled to −78° C. n-BuLi (27.5 mL, 1.6M in hexanes, 44 mmol) was added dropwise thereto for 10 minutes, and the mixture was stirred for 40 minutes. Then, the mixture was added dropwise to 20 mL of a THF solution of B(O-i-Pr)₃ (13.9 mL, 60 mmol), which had been cooled to 0° C., for 15 minutes. The mixture was stirred at the same temperature for 1 hour and then at room temperature for 17 hours. After completion of the reaction, the solvent was removed therefrom under reduced pressure, and the reaction product was separated and purified by column chromatography using ethyl acetate and hexane to obtain Intermediate 7-a (5.6 g, 68%).

The obtained compound was identified by ESI-LCMS as Intermediate 7-a. (ESI-LCMS: [M]⁺: C₁₅H₁₄BNO₄. 283.10)

### Synthesis of Intermediate 7-b

In an argon atmosphere, 4 mL of an ethanol (EtOH) solution of Intermediate 7-a (2.3 g, 8.0 mmol) and 3.6 mL of a 2M aqueous sodium carbonate (Na₂CO₃) solution were added to 10 mL of a toluene solution of 4-iodobenzo[c][1,2,5]oxadiazole (1.0 g, 4.0 mmol), and the resultant mixture was purged with nitrogen while being degassed. Then, Pd(PPh₃)₄ (0.3 g, 0.3 mmol) was added thereto, and the mixture was stirred at 80° C. for 12 hours. After cooling, an extraction process was performed thereon using water (500 mL) and ethyl acetate (300 mL) to collect an organic layer, which was then dried using anhydrous MgSO₄ and filtered. The filtered solution was placed under reduced pressure to remove the solvent therefrom, and the obtained solid was separated and purified by column chromatography using CH₂Cl₂ and hexane to obtain Intermediate 7-b (0.9 g, 2.5 mmol, 62%).

The obtained compound was identified by ESI-LCMS as Intermediate 7-b. (ESI-LCMS: [M]⁺: C₂₁H₁₅N₃O₃. 357.11)

### Synthesis of Intermediate 7-c

In an argon atmosphere, Intermediate 7-b (0.8 g, 2.3 mmol) was dissolved in 50 mL of THF, and the resultant mixture was cooled to −78° C. n-BuLi (1.9 mL, 1.6M in hexanes, 3.0 mmol) was added thereto, and the mixture was stirred for 30 minutes. Then, DMF (0.2 mL, 3.0 mmol) was added thereto, and the mixture was stirred at the same temperature for 30 minutes and then at room temperature for 10 hours. After cooling, an extraction process was performed thereon using water (500 mL) and ethyl acetate (300 mL) to collect an organic layer, which was then dried using anhydrous MgSO₄ and filtered. The filtered solution was placed under reduced pressure to remove the solvent therefrom, and the obtained solid was separated and purified by column chromatography using CH₂Cl₂ and hexane to obtain Intermediate 7-c (0.5 g, 1.3 mmol, 59%).

The obtained compound was identified by ESI-LCMS as Intermediate 7-c. (ESI-LCMS: [M]⁺: C₂₂H₁₅N₃O₄. 385.11)

### Synthesis of Compound 7

In an argon atmosphere, Intermediate 7-c (0.15 g, 0.4 mmol) was dissolved in 5 mL of chloroform (CHCl₃), malononitrile (0.03 g, 0.5 mmol), 5 mL of a methanol (CH₃OH) solution, and 1 mL of Et₃N were added thereto, and the mixture was stirred at room temperature for 35 minutes and then concentrated under reduced pressure.

The obtained solid was separated and purified by column chromatography using CH₂Cl₂ and hexane to obtain Compound 7 (0.12 g, 0.27 mmol, 60%).

The obtained compound was identified by ESI-LCMS as Compound 7. (ESI-LCMS: [M]⁺: C₂₅H₁₅N₅O₃. 433.12)

### Synthesis Example 3 (Synthesis of Compound 9)

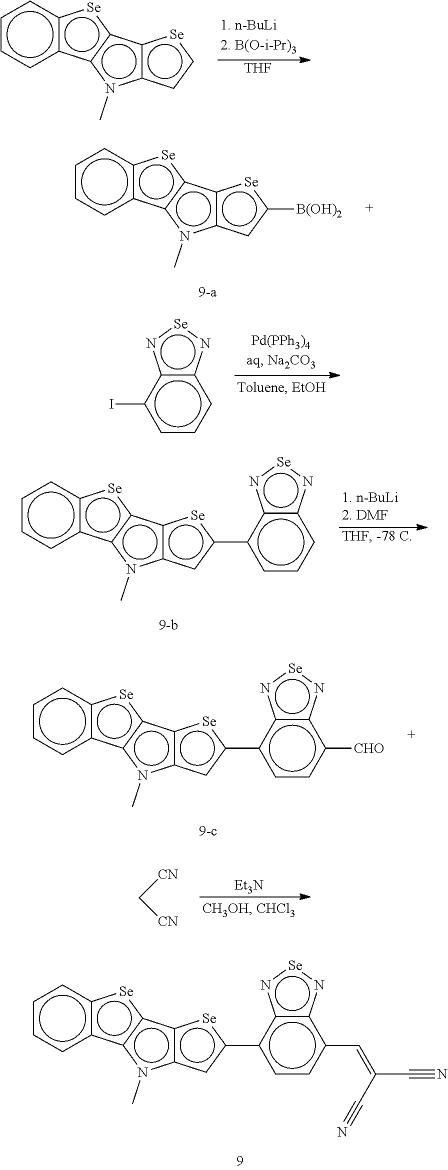

### Synthesis of Intermediate 9-a

In an argon atmosphere, 4-methyl-4H-benzo[4,5]selenopheno[3,2-b]selenopheno[2,3-d]pyrrole (13.6 mL, 40 mmol) was dissolved in 40 mL of THF, and the resultant mixture was cooled to −78° C. n-BuLi (27.5 mL, 1.6M in hexanes, 44 mmol) was added dropwise thereto for 10 minutes, and the mixture was stirred for 40 minutes. Then, the mixture was added dropwise to 20 mL of a THF solution of B(O-i-Pr)₃ (13.9 mL, 60 mmol), which had been cooled to 0° C., for 15 minutes. The mixture was stirred at the same temperature for 1 hour and then at room temperature for 17 hours. After completion of the reaction, the solvent was removed therefrom under reduced pressure, and the reaction product was separated and purified by column chromatography using ethyl acetate and hexane to obtain Intermediate 9-a (7.6 g, 66

The obtained compound was identified by ESI-LCMS as Intermediate 9-a. (ESI-LCMS: [M]⁺: C₁₃H₁₀BNO₂Se₂. 382.91)

### Synthesis of Intermediate 9-b

In an argon atmosphere, 4 mL of an ethanol (EtOH) solution of Intermediate 9-a (3.1 g, 8.0 mmol) and 3.6 mL of a 2M aqueous sodium carbonate (Na₂CO₃) solution were added to 10 mL of a toluene solution of 4-iodobenzo[c][1,2,5]selenadiazole (1.2 g, 4.0 mmol), and the resultant mixture was purged with nitrogen while being degassed. Then, Pd(PPh₃)₄ (0.3 g, 0.3 mmol) was added thereto, and the mixture was stirred at 80° C. for 12 hours. After cooling, an extraction process was performed thereon using water (500 mL) and ethyl acetate (300 mL) to collect an organic layer, which was then dried using anhydrous MgSO₄ and filtered. The filtered solution was placed under reduced pressure to remove the solvent therefrom, and the obtained solid was separated and purified by column chromatography using CH₂Cl₂ and hexane to obtain Intermediate 9-b (1.3 g, 2.5 mmol, 61%).

The obtained compound was identified by ESI-LCMS as Intermediate 9-b. (ESI-LCMS: [M]⁺: C₁₉H₁₁N₃Se₃. 520.84)

### Synthesis of Intermediate 9-c

In an argon atmosphere, Intermediate 9-b (1.2 g, 2.3 mmol) was dissolved in 50 mL of THF, and the resultant mixture was cooled to −78° C. n-BuLi (1.9 mL, 1.6M in hexanes, 3.0 mmol) was added thereto, and the mixture was stirred for 30 minutes. Then, DMF (0.2 mL, 3.0 mmol) was added thereto, and the mixture was stirred at the same temperature for 30 minutes and then at room temperature for 10 hours. After cooling, an extraction process was performed thereon using water (500 mL) and ethyl acetate (300 mL) to collect an organic layer, which was then dried using anhydrous MgSO₄ and filtered. The filtered solution was placed under reduced pressure to remove the solvent therefrom, and the obtained solid was separated and purified by column chromatography using CH₂Cl₂ and hexane to obtain Intermediate 9-c (0.7 g, 1.3 mmol, 59%).

The obtained compound was identified by ESI-LCMS as Intermediate 9-c. (ESI-LCMS: [M]⁺: C₂₀H₁₁N₃OSe₃. 548.84)

### Synthesis of Compound 9

In an argon atmosphere, Intermediate 9-c (0.22 g, 0.4 mmol) was dissolved in 5 mL of chloroform (CHCl₃), malononitrile (0.03 g, 0.5 mmol), 5 mL of a methanol (CH₃OH) solution, and 1 mL of Et₃N were added thereto, and the resultant mixture was stirred at room temperature for 35 minutes and then concentrated under reduced pressure. The obtained solid was separated and purified by column chromatography using CH₂Cl₂ and hexane to obtain Compound 9 (0.16 g, 0.27 mmol, 60%).

The obtained compound was identified by ESI-LCMS as Compound 9. (ESI-LCMS: [M]⁺: C₂₃H₁₁N₅Se₃. 596.85)

### Synthesis Example 4 (Synthesis of Compound 16)

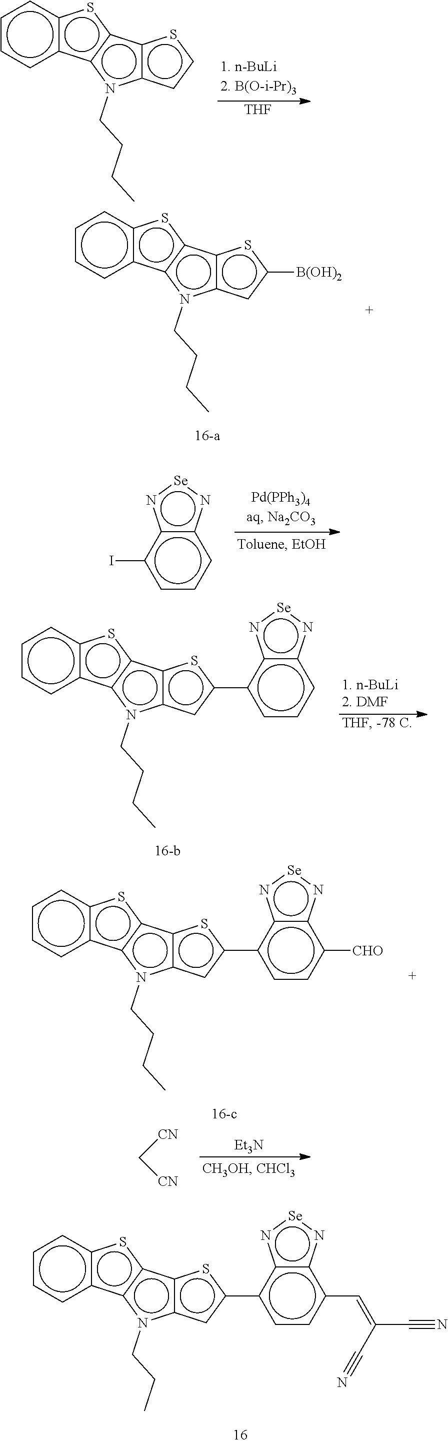

### Synthesis of Intermediate 16-a

In an argon atmosphere, 4-butyl-4H-benzo[4,5]thieno[3,2-b]thieno[2,3-d]pyrrole (11.4 mL, 40 mmol) was dissolved in 40 mL of THF, and the resultant mixture was cooled to −78° C. n-BuLi (27.5 mL, 1.6M in hexanes, 44 mmol) was added dropwise thereto for 10 minutes, and the mixture was stirred for 40 minutes. Then, the mixture was added dropwise to 20 mL of a THF solution of B(O-i-Pr)₃ (13.9 mL, 60 mmol), which had been cooled to 0° C., for 15 minutes. The mixture was stirred at the same temperature for 1 hour and then at room temperature for 17 hours. After completion of the reaction, the solvent was removed therefrom under reduced pressure, and the reaction product was separated and purified by column chromatography using ethyl acetate and hexane to obtain Intermediate 16-a (6.6 g, 65

The obtained compound was identified by ESI-LCMS as Intermediate 16-a. (ESI-LCMS: [M]⁺: C₁₆H₁₆BNO₂S₂. 329.07)

### Synthesis of Intermediate 16-b

In an argon atmosphere, 4 mL of an ethanol (EtOH) solution of Intermediate 16-a (2.6 g, 8.0 mmol) and 3.6 mL of a 2M aqueous sodium carbonate (Na₂CO₃) solution were added to 10 mL of a toluene solution of 4-iodobenzo[c][1,2,5]selenadiazole (1.2 g, 4.0 mmol), and the resultant mixture was purged with nitrogen while being degassed. Then, Pd(PPh₃)₄ (0.3 g, 0.3 mmol) was added thereto, and the mixture was stirred at 80° C. for 12 hours. After cooling, an extraction process was performed thereon using water (500 mL) and ethyl acetate (300 mL) to collect an organic layer, which was then dried using anhydrous MgSO₄ and filtered. The filtered solution was placed under reduced pressure to remove the solvent therefrom, and the obtained solid was separated and purified by column chromatography using CH₂Cl₂ and hexane to obtain Intermediate 16-b (1.2 g, 2.5 mmol, 63%).

The obtained compound was identified by ESI-LCMS as Intermediate 16-b. (ESI-LCMS: [M]⁺: C₂₂H₁₇N₃S₂Se. 467.00)

### Synthesis of Intermediate 16-c

In an argon atmosphere, Intermediate 16-b (1.1 g, 2.3 mmol) was dissolved in 50 mL of THF, and the resultant mixture was cooled to −78° C. n-BuLi (1.9 mL, 1.6M in hexanes, 3.0 mmol) was added thereto, and the mixture was stirred for 30 minutes. Then, DMF (0.2 mL, 3.0 mmol) was added thereto, and the mixture was stirred at the same temperature for 30 minutes and then at room temperature for 10 hours. After cooling, an extraction process was performed thereon using water (500 mL) and ethyl acetate (300 mL) to collect an organic layer, which was then dried using anhydrous MgSO₄ and filtered. The filtered solution was placed under reduced pressure to remove the solvent therefrom, and the obtained solid was separated and purified by column chromatography using CH₂Cl₂ and hexane to obtain Intermediate 16-c (0.6 g, 1.3 mmol, 57%).

The obtained compound was identified by ESI-LCMS as Intermediate 16- [symbol] . (ESI-LCMS: [M]⁺: C₂₃H₁₇N₃OS₂Se. 495.00)

### Synthesis of Compound 16

In an argon atmosphere, Intermediate 16-c (0.20 g, 0.4 mmol) was dissolved in 5 mL of chloroform (CHCl₃), malononitrile (0.03 g, 0.5 mmol), 5 mL of a methanol (CH₃OH) solution, and 1 mL of Et₃N were added thereto, and the resultant mixture was stirred at room temperature for 35 minutes and then concentrated under reduced pressure. The obtained solid was separated and purified by column chromatography using CH₂Cl₂ and hexane to obtain Compound 16 (0.15 g, 0.27 mmol, 59%).

The obtained compound was identified by ESI-LCMS as Compound 16. (ESI-LCMS: [M]⁺: C₂₆H₁₇N₅S₂Se. 543.01)

### Synthesis Example 5 (Synthesis of Compound 20)

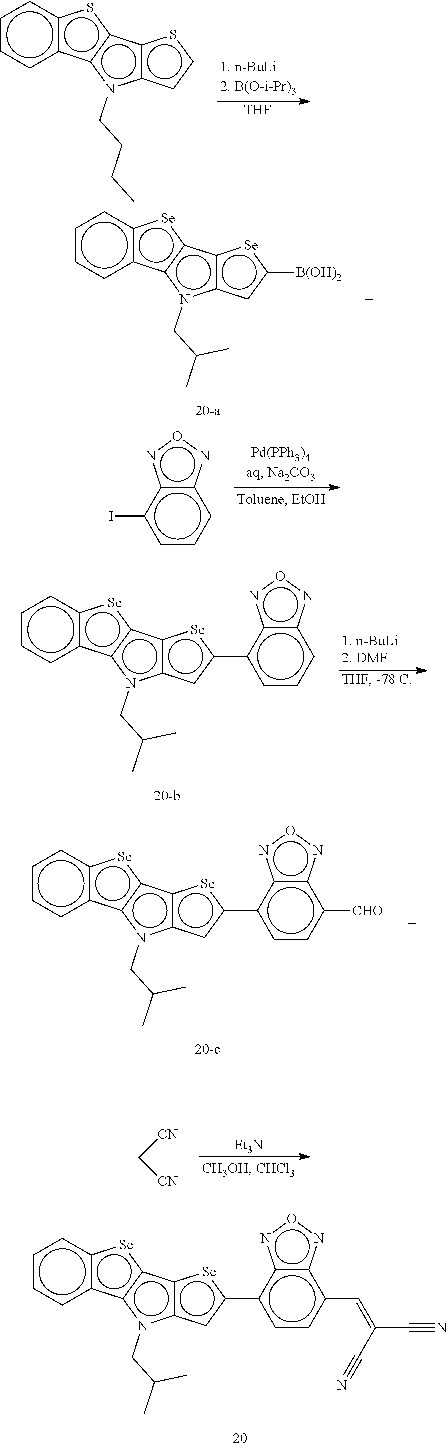

### Synthesis of Intermediate 20-a

In an argon atmosphere, 4-butyl-4H-benzo[4,5]thieno[3,2-b]thieno[2,3-d]pyrrole (11.4 mL, 40 mmol) was dissolved in 40 mL of THF, and the mixture was cooled to −78° C. n-BuLi (27.5 mL, 1.6M in hexanes, 44 mmol) was added dropwise thereto for 10 minutes, and the resultant mixture was stirred for 40 minutes. Then, the mixture was added dropwise to 20 mL of a THF solution of B(O-i-Pr)₃ (13.9 mL, 60 mmol), which had been cooled to 0° C., for 15 minutes. The mixture was stirred at the same temperature for 1 hour and then at room temperature for 17 hours. After completion of the reaction, the solvent was removed therefrom under reduced pressure, and the reaction product was separated and purified by column chromatography using ethyl acetate and hexane to obtain Intermediate 20-a (8.5 g, 65

The obtained compound was identified by ESI-LCMS as Intermediate 20-a. (ESI-LCMS: [M]⁺: C₁₆H₁₆BNO₂Se₂. 424.96)

### Synthesis of Intermediate 20-b

In an argon atmosphere, 4 mL of an ethanol (EtOH) solution of Intermediate 20-a (3.4 g, 8.0 mmol) and 3.6 mL of a 2M aqueous sodium carbonate (Na₂CO₃) solution were added to 10 mL of a toluene solution of 4-iodobenzo[c][1,2,5]oxadiazole (1.0 g, 4.0 mmol), and the resultant mixture was purged with nitrogen while being degassed. Then, Pd(PPh₃)₄ (0.3 g, 0.3 mmol) was added thereto, and the mixture was stirred at 80° C. for 12 hours. After cooling, an extraction process was performed thereon using water (500 mL) and ethyl acetate (300 mL) to collect an organic layer, which was then dried using anhydrous MgSO₄ and filtered. The filtered solution was placed under reduced pressure to remove the solvent therefrom, and the obtained solid was separated and purified by column chromatography using CH₂Cl₂ and hexane to obtain Intermediate 20-b (1.2 g, 2.5 mmol, 60%).

The obtained compound was identified by ESI-LCMS as Intermediate 20-b. (ESI-LCMS: [M]⁺: C₂₂H₁₇N₃OSe₂. 498.97)

### Synthesis of Intermediate 20-c

In an argon atmosphere, Intermediate 20-b (1.1 g, 2.2 mmol) was dissolved in 50 mL of THF, and the resultant mixture was cooled to −78° C. n-BuLi (1.9 mL, 1.6M in hexanes, 3.0 mmol) was added thereto, and the mixture was stirred for 30 minutes. Then, DMF (0.2 mL, 3.0 mmol) was added thereto, and the mixture was stirred at the same temperature for 30 minutes and then at room temperature for 10 hours. After cooling, an extraction process was performed thereon using water (500 mL) and ethyl acetate (300 mL) to collect an organic layer, which was then dried using anhydrous MgSO₄ and filtered. The filtered solution was placed under reduced pressure to remove the solvent therefrom, and the obtained solid was separated and purified by column chromatography using CH₂Cl₂ and hexane to obtain Intermediate 20-c (0.7 g, 1.3 mmol, 60%).

The obtained compound was identified by ESI-LCMS as Intermediate 20-c. (ESI-LCMS: [M]⁺: C₂₃H₁₇N₃O₂Se₂. 526.97)

### Synthesis of Compound 20

In an argon atmosphere, Intermediate 20-c (0.21 g, 0.4 mmol) was dissolved in 5 mL of chloroform (CHCl₃), malononitrile (0.03 g, 0.5 mmol), 5 mL of a methanol (CH₃OH) solution, and 1 mL of Et₃N were added thereto, and the resultant mixture was stirred at room temperature for 35 minutes and then concentrated under reduced pressure. The obtained solid was separated and purified by column chromatography using CH₂Cl₂ and hexane to obtain Compound 20 (0.15 g, 0.27 mmol, 59%).

The obtained compound was identified by ESI-LCMS as Compound 20. (ESI-LCMS: [M]⁺: C₂₆H₁₇N₅OSe₂. 574.98)

### Comparative Example 1

As an anode, a glass substrate with 15 Ω/cm² (1,200 Å) ITO (manufactured by Corning. Inc.,) formed thereon was cut to a size of 50 mm×50 mm×0.7 mm, and sonicated with isopropyl alcohol and pure water, each for 5 minutes. Then, ultraviolet light was irradiated for 30 minutes thereto, and ozone was exposed thereto for cleaning. Subsequently, the resultant glass substrate was mounted on a vacuum deposition apparatus.

2-TNATA was vacuum-deposited on the anode to form a hole injection layer having a thickness of 100 Å. 4,4′-bis[N-(1-naphthyl)-N-phenylamino]biphenyl (hereinafter, referred to as NPB) was vacuum-deposited on the hole injection layer to form a hole transport layer having a thickness of 1,250 Å.

Boron subphthalocyanine chloride (hereinafter, referred to as SubPC) was vacuum-deposited on the hole transport layer to form a first layer included in a photoactive layer and having a thickness of 200 Å.

Fullerene was vacuum-deposited on the first layer to form a second layer included in the photoactive layer and having a thickness of 250 Å.

BAlq was vacuum-deposited on the second layer to form a hole blocking layer having a thickness of 50 Å, and ET1 was vacuum-deposited on the hole blocking layer to form an electron transport layer having a thickness of 300 Å. Liq was vacuum-deposited on the electron transport layer to form an electron injection layer having a thickness of 10 Å. AgMg was vacuum-deposited on the electron injection layer to form a cathode having a thickness of 100 Å, thereby completing the manufacture of an opto-electronic device:

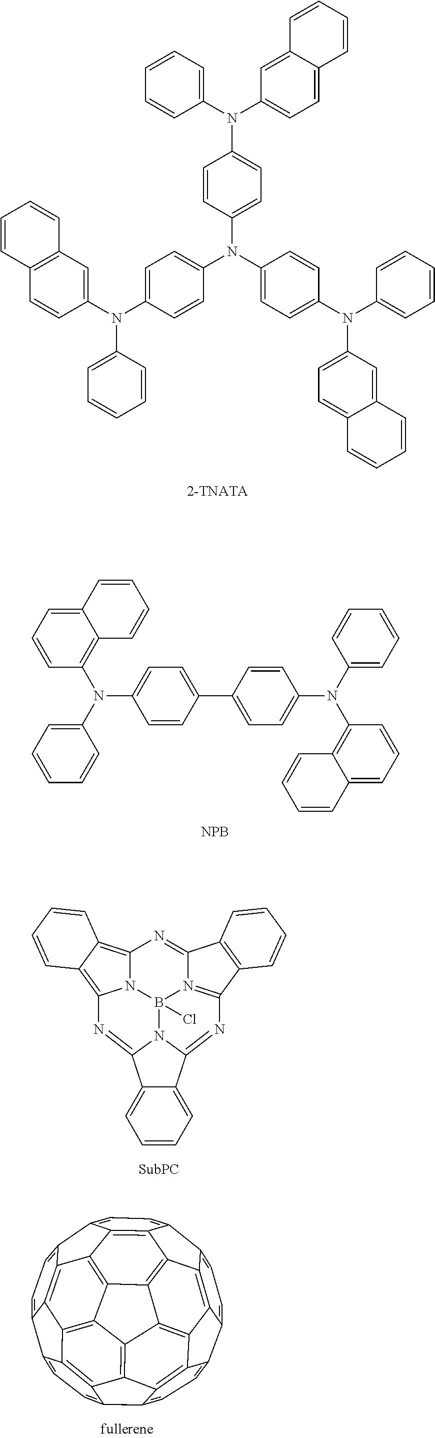

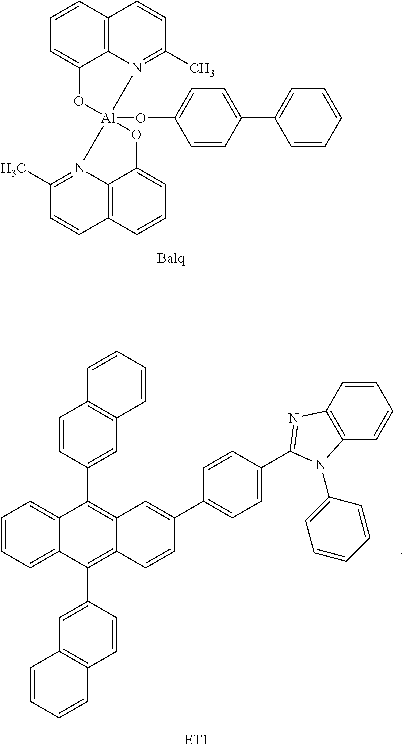

### Comparative Examples 2 to 5 and Examples 1 to 5

Opto-electronic devices were manufactured in substantially the same manner as in Comparative Example 1, except that, when forming a first layer included in a photoactive layer, the compounds shown in Table 1 were each used instead of SubPC.

### Evaluation Example 1

To evaluate the characteristics of the compounds respectively used in the first layers in Examples 1 to 5 and Comparative Examples 1 to 5, the highest occupied molecular orbital (HOMO) energy level, LUMO energy level, maximum absorption wavelength (λ_{abs}), oscillator strength (OSC), exciton binding energy (E_{b}) and molecular weight (MW) of each compound were measured, and the results are shown in Table 1.

In evaluating the above characteristics, the HOMO and LUMO energy levels and oscillator strength in a structurally optimized state and an excited state were evaluated by performing quantum simulation at a level of B3LYP/6-311 G** based on the time-dependent density functional theory (TD-DFT) method by using the Gaussian program.

TABLE 1

| | | HOMO | LUMO | λ_{abs} | OSC | E_{b} | MW |
|---|---|---|---|---|---|---|---|
| | Donor | [eV] | [eV] | [nm] | [−] | [eV] | [g/mol] |
| Example 1 | Compound | −5.31 | −3.42 | 630 | 0.65 | 0.15 | 497 |
| | 4 | | | | | | |
| Example 2 | Compound | −5.37 | −3.50 | 630 | 0.71 | 0.13 | 435 |
| | 7 | | | | | | |
| Example 3 | Compound | −5.26 | −3.42 | 636 | 0.71 | 0.14 | 599 |
| | 9 | | | | | | |
| Example 4 | Compound | −5.26 | −3.40 | 633 | 0.68 | 0.14 | 545 |
| | 16 | | | | | | |
| Example 5 | Compound | −5.42 | −3.56 | 634 | 0.64 | 0.15 | 577 |
| | 20 | | | | | | |
| Comparative | SubPc | −5.55 | −2.83 | 590 | 0.62 | 0.22 | 430 |
| Example 1 | | | | | | | |
| Comparative | SubNC | −5.08 | −2.75 | 650 | 0.62 | 0.21 | 580 |
| Example 2 | | | | | | | |
| Comparative | Compound | −5.56 | −3.37 | 566 | 0.79 | 0.13 | 464 |
| Example 3 | A | | | | | | |
| Comparative | Compound | −5.69 | −3.55 | 580 | 0.83 | 0.10 | 445 |
| Example 4 | B | | | | | | |
| Comparative | Compound | −5.31 | −3.06 | 551 | 0.29 | 0.32 | 580 |
| Example 5 | C | | | | | | |

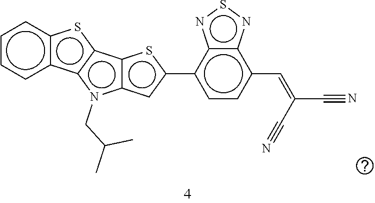

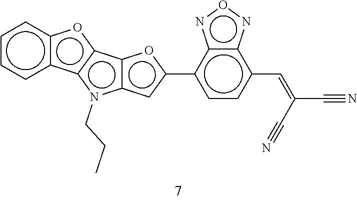

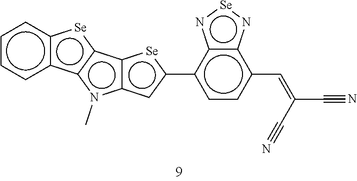

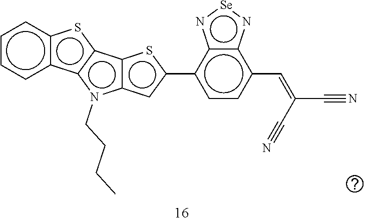

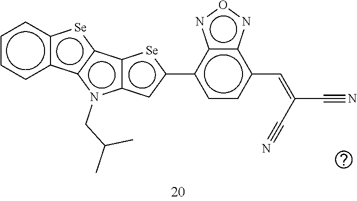

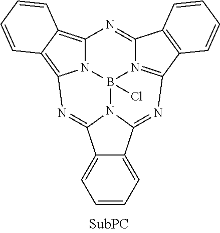

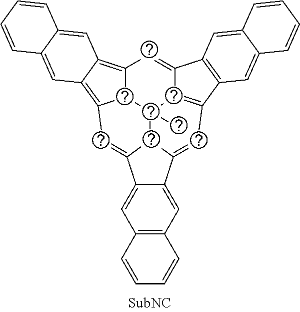

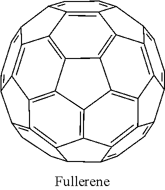

Compound A 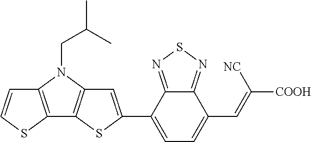 [SMILES: CC(C)Cn1c2ccsc2c2sc(-c3ccc(/C=C(\C#N)C(=O)O)c4nsnc34)cc21]
Compound B 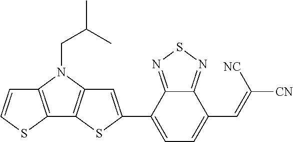 [SMILES: CC(C)Cn1c2ccsc2c2sc(-c3ccc(C=C(C#N)C#N)c4nsnc34)cc21]
Compound C 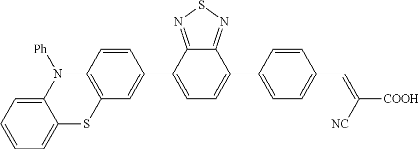 [SMILES: N#C/C(=C\c1ccc(-c2ccc(-c3ccc4c(c3)Sc3ccccc3N4c3ccccc3)c3nsnc23)cc1)C(=O)O]
Evaluation Example 2

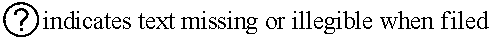

To evaluate the characteristics of the opto-electronic devices manufactured in Examples 1 to 5 and Comparative Examples 1 to 5, the external quantum efficiency (EQE) and dark current density (J_{dark}) of each opto-electronic device were measured, and the results are shown in Table 2.

In measuring the external quantum efficiency (EQE), each of the opto-electronic devices manufactured in Examples 1 to 5 and Comparative Examples 1 to 5 was irradiated with light (wavelength of 630 nm) by using an external quantum efficiency meter (K3100, McScience, Korea), a generated current value was measured by using an ammeter (Keithley, Tektronix, USA), and the measured current value was calculated as an external quantum efficiency (EQE) value.

The dark current density (J_{dark}) was measured by using an ammeter (Keithley, Tektronix, USA) in a state in which a voltage was applied to an anode at a dark current density with a reverse bias of −3 V by using electro-optical characteristics evaluation equipment (K3100, McScience, Korea).

TABLE 2

| | | | EQE | J_{dark} |
|---|---|---|---|---|
| | Donor | Acceptor | [%] | [mA/cm²] |
| Example 1 | Compound 4 | Fullerene | 34 | 3.3 × 10⁻⁶ |
| Example 2 | Compound 7 | Fullerene | 36 | 3.1 × 10⁻⁶ |
| Example 3 | Compound 9 | Fullerene | 33 | 3.4 × 10⁻⁶ |
| Example 4 | Compound 16 | Fullerene | 34 | 3.5 × 10⁻⁶ |
| Example 5 | Compound 20 | Fullerene | 34 | 3.8 × 10⁻⁶ |
| Comparative | SubPc | Fullerene | 7 | 3.5 × 10⁻⁶ |
| Example 1 | | | | |
| Comparative | SubNC | Fullerene | 20 | 4.3 × 10⁻⁶ |
| Example 2 | | | | |
| Comparative | Compound A | Fullerene | 18 | 3.2 × 10⁻⁶ |
| Example 3 | | | | |
| Comparative | Compound B | Fullerene | 22 | 2.8 × 10⁻⁶ |
| Example 4 | | | | |
| Comparative | Compound C | Fullerene | 4 | 8.3 × 10⁻⁶ |
| Example 5 | | | | |

Referring to Table 1, the opto-electronic devices according to Examples 1 to 5, which had a maximum absorption wavelength coinciding with the center of a red wavelength, showed improved light absorption efficiency compared to the opto-electronic devices according to Comparative Examples 1 to 5, which had a maximum absorption wavelength deviating from the center of a red wavelength. Referring to Table 2, the opto-electronic devices according to Examples 1 to 5 have high external quantum efficiency (EQE) and low dark current density (J_{dark}) compared to the opto-electronic devices according to Comparative Examples 1 to 5. Accordingly, the opto-electronic devices according to Examples 1 to 5 have excellent opto-electronic characteristics.

According to the one or more embodiments, an organic compound represented by Formula 1 may have excellent light absorption efficiency for a set or specific wavelength. Accordingly, an opto-electronic device including the organic compound may have high light absorption efficiency. An electronic apparatus including the opto-electronic device and an electronic device using the electronic apparatus may have improved quality.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the claims, and equivalents thereof.

What is claimed is:

1. An opto-electronic device comprising:
a first electrode;
a second electrode facing the first electrode;
a photoactive layer arranged between the first electrode and the second electrode; and
an organic compound represented by Formula 1: 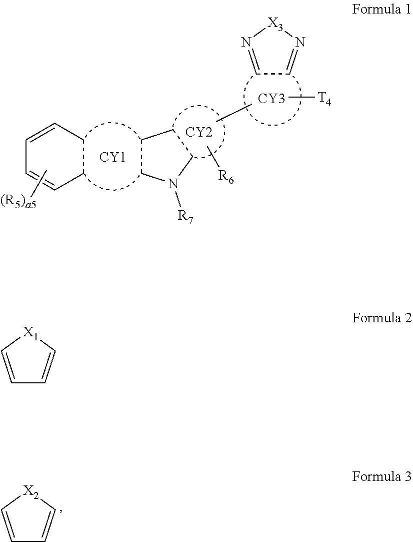
wherein, in Formulae 1 to 3,
CY1 is a group represented by Formula 2,
CY2 is a group represented by Formula 3,
CY3 is a C₃-C₆₀ carbocyclic group or a C₁-C₆₀ heterocyclic group,
X₁ is O, S, Se, N(R₁), or P(═O)R₁,
X₂ is O, S, Se, N(R₂), or P(═O)R₂,
X₃ is O, S, Se, N(R₃), or P(═O)R₃,
T₄ is *-(L₄)_{b4}-(R₄)ₐ₄,
L₄ is a single bond, a C₁-C₆₀ alkylene group unsubstituted or substituted with at least one R₁₀ₐ, a C₂-C₆₀ alkenylene group unsubstituted or substituted with at least one R₁₀ₐ, or a C₂-C₆₀ alkynylene group unsubstituted or substituted with at least one R₁₀ₐ,
b4 is an integer from 0 to 3,
R₁ to R₇ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
a4 and a5 are each independently an integer from 0 to 4,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —CI, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
Q₁ to Q₃, Q₁₁ to Q₁₃, Q₂₁ to Q₂₃, and Q₃₁ to Q₃₃ are each independently: hydrogen; deuterium; —F; —CI; —Br; —I; a hydroxyl group; a cyano group; a nitro group; a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, a hydroxyl group, a cyano group, a nitro group, or any combination thereof; a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a C₃-C₆₀ carbocyclic group, a C₁-C₆₀ heterocyclic group, or any combination thereof; a C₇-C₆₀ arylalkyl group; or a C₂-C₆₀ heteroarylalkyl group, and
* indicates a binding site to CY3.

2. The opto-electronic device of claim 1, further comprising: a hole transport region arranged between the first electrode and the photoactive layer; and an electron transport region arranged between the photoactive layer and the second electrode,
wherein the photoactive layer comprises the organic compound.

3. The opto-electronic device of claim 2, wherein the photoactive layer comprises: a first layer adjacent to the hole transport region; and a second layer adjacent to the electron transport region, and
the first layer comprises the organic compound.

4. The opto-electronic device of claim 3, wherein the first layer is in direct contact with the hole transport region.

5. An electronic apparatus comprising the opto-electronic device of claim 1.

6. The electronic apparatus of claim 5, further comprising:
a thin-film transistor electrically connected to the first electrode;
an emission layer arranged between the first electrode and the second electrode and not overlapping the photoactive layer; and
a color filter, a color conversion layer, a touch screen layer, a polarizing layer, or any combination thereof.

7. An electronic device comprising the electronic apparatus of claim 5, wherein the electronic device is one selected from a flat panel display, a curved display, a computer monitor, a medical monitor, a television, a billboard, an indoor or outdoor lighting and/or signal light, a head-up display, a fully or partially transparent display, a flexible display, a rollable display, a foldable display, a stretchable display, a laser printer, a telephone, a mobile phone, a tablet, a phablet, a personal digital assistant, a wearable device, a laptop computer, a digital camera, a camcorder, a viewfinder, a microdisplay, a three-dimensional display, a virtual or augmented-reality display, a vehicle, a video wall including multiple displays tiled together, a theater or stadium screen, a phototherapy device, and a signboard.

8. An organic compound represented by Formula 1: 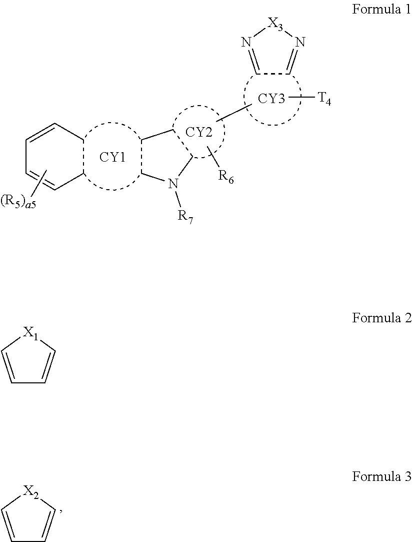
wherein, in Formulae 1 to 3,
CY1 is a group represented by Formula 2,
CY2 is a group represented by Formula 3,
CY3 is a C₃-C₆₀ carbocyclic group or a C₁-C₆₀ heterocyclic group,
X₁ is O, S, Se, N(R₁), or P(═O)R₁,
X₂ is O, S, Se, N(R₂), or P(═O)R₂,
X₃ is O, S, Se, N(R₃), or P(═O)R₂,
T₄ is *-(L₄)_{b4}-(R₄)ₐ₄,
L₄ is a single bond, a C₁-C₆₀ alkylene group unsubstituted or substituted with at least one R₁₀ₐ, a C₂-C₆₀ alkenylene group unsubstituted or substituted with at least one R₁₀ₐ, or a C₂-C₆₀ alkynylene group unsubstituted or substituted with at least one R₁₀ₐ,
b4 is an integer from 0 to 3,
R₁ to R₇ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
a4 and a5 are each independently an integer from 0 to 4,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
Q₁ to Q₃, Q₁₁ to Q₁₃, Q₂₁ to Q₂₃ and Q₃₁ to Q₃₃ are each independently: hydrogen; deuterium; —F; —CI; —Br; —I; a hydroxyl group; a cyano group; a nitro group; a C₁-C₆₀ alkyl group, a C₂-C₆₀ alkenyl group, a C₂-C₆₀ alkynyl group or a C₁-C₆₀ alkoxy group, each unsubstituted or substituted with deuterium, —F, a hydroxyl group, a cyano group, a nitro group, or any combination thereof; a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a C₃-C₆₀ carbocyclic group, a C₁-C₆₀ heterocyclic group, or any combination thereof; a C₇-C₆₀ arylalkyl group; or a C₂-C₆₀ heteroarylalkyl group, and
* indicates a binding site to CY3.

9. The organic compound of claim 8, wherein the organic compound is represented by one selected from Formulae 2-1 and 2-2: 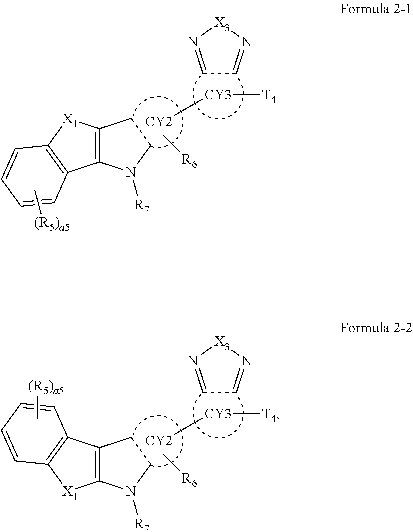
wherein, in Formulae 2-1 and 2-2,
CY2, CY3, X₁ to X₃, T₄, L₄, b4, R₁ to R₇, a4, and a5 are each as defined with respect to Formulae 1 to 3.

10. The organic compound of claim 8, wherein the organic compound is represented by one selected from Formulae 3-1 to 3-4: 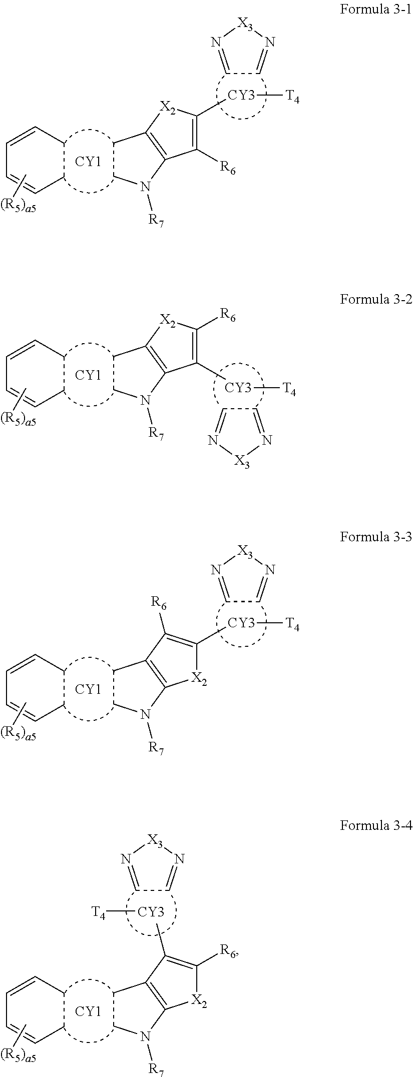
wherein, in Formulae 3-1 to 3-4,
CY1, CY3, X₁ to X₃, T₄, L₄, b4, R₁ to R₇, a4, and a5 are each as defined with respect to Formulae 1 to 3.

11. The organic compound of claim 8, wherein the organic compound is represented by one selected from Formulae 1-1 to 1-8: 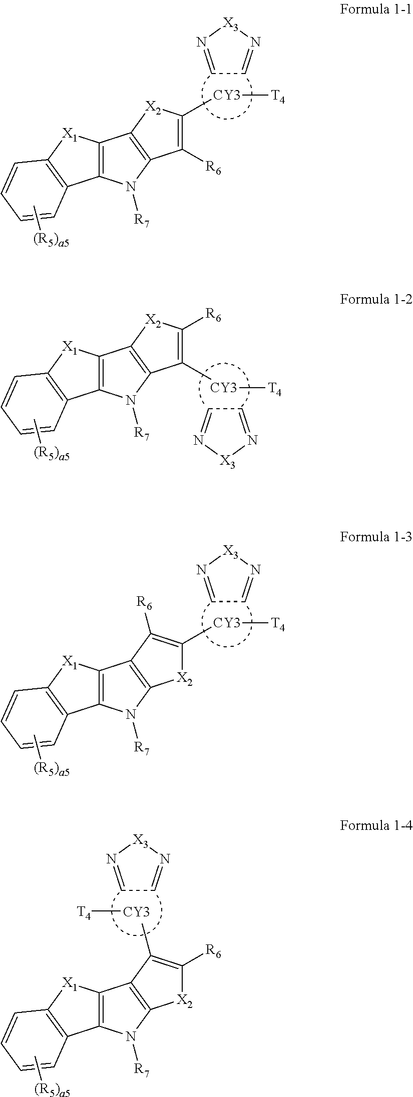 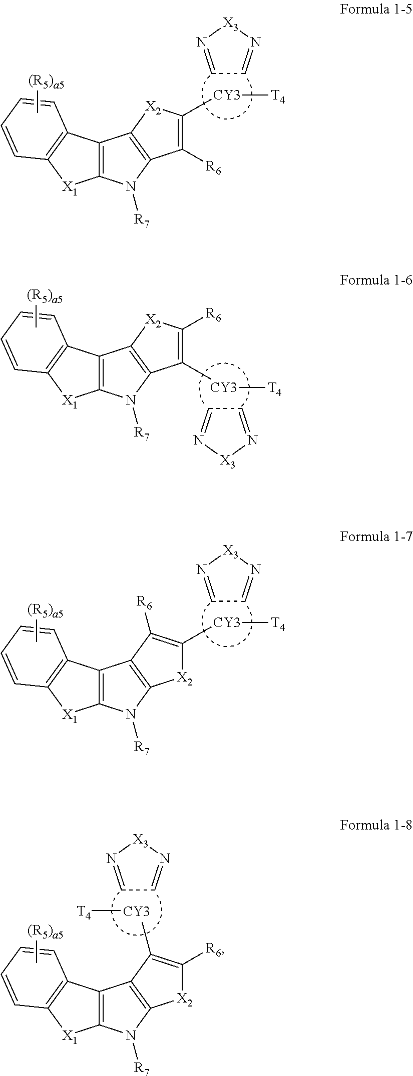
wherein, in Formulae 1-1 to 1-8,
CY3, X₁ to X₃, T₄, L₄, b4, R₁ to R₇, a4, and a5 are each as defined with respect to Formulae 1 to 3.

12. The organic compound of claim 8, wherein, in Formula 1, CY3 is a benzene group, a naphthalene group, a pyridine group, a pyrazine group, a pyrimidine group, a pyridazine group, a quinoline group, or an isoquinoline group.

13. The organic compound of claim 8, wherein Formula 1 is represented by one selected from Formulae 4-1 to 4-6: 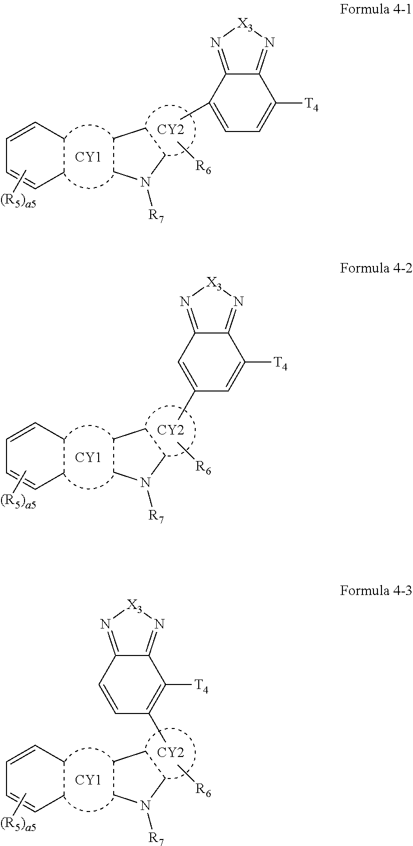 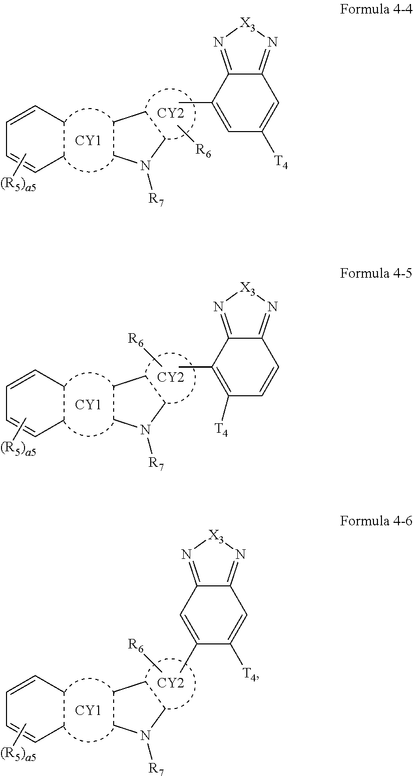
wherein, in Formulae 4-1 to 4-6,
CY1, CY2, X₁ to X₃, T₄, L₄, b4, R₁ to R₇, a4, and a5 are each as defined with respect to Formulae 1 to 3.

14. The organic compound of claim 8, wherein, in Formulae 1 to 3, X₁ to X₃ are each independently O, S, or Se.

15. The organic compound of claim 8, wherein, in Formulae 1 to 3, X₁ and X₂ are identical to each other.

16. The organic compound of claim 8, wherein, in Formula 1, L₄ is a single bond or a C₂-C₆₀ alkenylene group unsubstituted or substituted with at least one R₁₀ₐ.

17. The organic compound of claim 8, wherein, in Formulae 1 to 3, R₁ to R₇ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, or a C₂-C₆₀ alkynyl group unsubstituted or substituted with at least one R₁₀ₐ.

18. The organic compound of claim 8, wherein, in Formula 1, T₄ is represented by Formula 5: 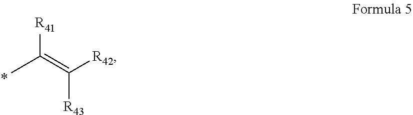
wherein, in Formula 5,
R₄₁ to R₄₃ are each independently as defined in connection with R₄ with respect to Formula 1, and
* indicates a binding site to CY3.

19. The organic compound of claim 18, wherein, in Formula 5, R₄₂ and R₄₃ are each a cyano group.

20. The organic compound of claim 8, wherein a maximum absorption wavelength of the organic compound is in a range of 615 nm to 645 nm.
